SEQUENCE LISTING (8 sequences)

SEQ ID NO: 1 - DNA, 18 bp, synthetic construct
  source 1..18: mol_type = other DNA
aattaaccctcactaaag

SEQ ID NO: 2 - DNA, 20 bp, synthetic construct
  source 1..20: mol_type = other DNA
tcataaaaaatttatttgct

SEQ ID NO: 3 - DNA, 23 bp, synthetic construct
  source 1..23: mol_type = other DNA
taatacgactcactatagggaga

SEQ ID NO: 4 - DNA, 10 bp, synthetic construct
  source 1..10: mol_type = other DNA
cactataggg

SEQ ID NO: 5-8 - skipped (4 entries)

(19) United States
(12) Patent Application Publication
(10) Pub. No.: US 2025/0230497 A1
(43) Pub. Date: Jul. 17, 2025

(54) METHODS FOR POLYNUCLEOTIDE SEQUENCING

(71) Applicant: Singular Genomics Systems, Inc., San Diego, CA (US)

(72) Inventors: Martin Maria Fabani, Encinitas, CA (US); Eli N. Glezer, Del Mar, CA (US); Sabrina Shore, San Diego, CA (US); Christopher Jen-Yue Wei, La Palma, CA (US)

(21) Appl. No.: 18/854,033

(22) PCT Filed: Apr. 7, 2023
(86) PCT No.: PCT/US2023/065538

§ 371 Date: Oct. 3, 2024

Related U.S. Application Data
(60) Provisional application No. 63/329,313, filed on Apr. 8, 2022.

(51) Int. Cl. C12Q 1/6874

(52) U.S. Cl. CPC C12Q 1/6874

(57) ABSTRACT

Provided herein, inter alia, are methods of making, amplifying, and sequencing tagged nucleic acid complements, compositions including barcoded adapters, and kits useful in obtaining long-range sequence data.

### CROSS-REFERENCES TO RELATED APPLICATIONS

This application claims the benefit of U.S. Provisional Application No. 63/329,313, filed Apr. 8, 2022, which is incorporated herein by reference in its entirety and for all purposes.

### SEQUENCE LISTING

The instant application contains a Sequence Listing which has been submitted electronically in ASCII format and is hereby incorporated by reference in its entirety. Said ASCII copy, created on Apr. 7, 2023, is named 051385-556001WO_SL_ST26.xml and is 5,385 bytes in size.

### BACKGROUND

Next-generation sequencing (NGS) platforms are available for the high-throughput, massively parallel sequencing of nucleic acids. Certain NGS sequencing methodologies make use of simultaneously sequencing millions of fragments of nucleic acids, resulting in a 50,000-fold drop in the costs associated with sequencing since its inception. Due to the read lengths of current NGS platforms, typically ranging in length from 35 to 300 base pairs, nucleic acid sequencing technologies may struggle with accurately mapping sequences having large structural variations, e.g., inversions and translocations, tandem repeat regions, distinguishing clinically relevant genes from pseudogenes, and haplotype reconstructions. Disclosed herein, inter alia, are solutions to these and other problems in the art.

### BRIEF SUMMARY

In an aspect is provided a method of sequencing a polynucleotide, the method comprising: contacting the polynucleotide comprising a first unique molecular identifier (UMI) sequence and a promoter sequence with an RNA polymerase and generating a plurality of RNA molecules, wherein each RNA molecule comprises a complement of said first UMI; fragmenting said plurality of RNA molecules to form a population of RNA nucleic acid fragments; attaching said population of RNA nucleic acid fragments to a solid support thereby forming a plurality of immobilized RNA nucleic acid fragments, and amplifying the plurality of immobilized RNA nucleic acid fragments to form amplification products immobilized to the solid support; hybridizing a sequencing primer to one or more of the amplification products and incorporating one or more nucleotides into the sequencing primer with a polymerase thereby forming one or more incorporated nucleotides; and detecting the one or more incorporated nucleotides thereby generating a sequencing read.

In an aspect is provided a method of sequencing a polynucleotide, the method comprising: a) contacting the polynucleotide with an amplification reagent and generating a first complement of said polynucleotide comprising an incorporated first cleavable site nucleotide at a first position; contacting the polynucleotide with said amplification reagent and generating a second complement of said polynucleotide comprising a second incorporated cleavable site nucleotide at a second position, wherein said first position and second position are different; wherein said amplification reagent comprises a polymerase, a plurality of native DNA nucleotides, and a plurality of cleavable site nucleotides; b) cleaving the first complement at the first position and cleaving the second complement at the second position to form nucleic acid fragments comprising a 3′ end; c) ligating an adapter to the 3′ end of each of the nucleic acid fragments thereby forming adapter fragments, wherein the adapter comprises a sequencing primer binding sequence; d) attaching said adapter fragments to immobilized primers on a solid support, and amplifying the adapter fragments to form amplification products immobilized to the solid support; and e) sequencing the amplification products, or complements thereof.

### BRIEF DESCRIPTION OF THE DRAWINGS

FIG. 1 is an illustration of an embodiment of the invention. A polynucleotide including a unique molecular identifier (UMI) is fragmented to generate a plurality of fragments of different lengths. Each fragment includes a UMI and is sequenced, optionally sequencing in both directions (i.e., sequencing the forward and reverse complement of the original fragment) using common paired-read or paired-end sequencing methods to generate a plurality of variable length sequencing reads, each read containing the fragment and the UMI, or complement thereof. The sequencing reads may then be bioinformatically grouped by the common UMI to reconstruct the original long polynucleotide molecule.

FIG. 2 illustrates an embodiment of the invention wherein sample polynucleotides (e.g., DNA fragments) are attached to adapters on one end (e.g., the 3′ end), followed by RNA transcription using a RNA polymerase, subsequent RNA fragmentation, a second adapter ligation on the other end (e.g., the 5′ end), amplification and/or subsequent detection. In embodiments, the ligated adapters include a T7 promoter site, a first primer binding site (P1), an optional constant region (not shown), and a UMI. Although only a single strand of the DNA fragment is shown, it is understood that the outlined method does not preclude the use of double-stranded DNA fragments. Next, the steps of linear amplification and RNA transcription are shown using T7 RNA polymerase (illustrated as a cloud-shaped object) to produce a plurality of single-stranded amplification products (note, only one amplification product is shown for clarity). Following RNA transcription, ligation of a second adapter including a second primer binding sequence (P2) is performed with T4 RNA ligase. The prepared library molecule may then by subjected to amplification by RT-PCR to generate DNA (i.e., cDNA) products. The resulting RT-PCR products (not shown) include a single common UMI that may be used to reconstruct the original sample polynucleotides, as shown in FIG. 1.

FIGS. 3A-3B illustrate an embodiment wherein double-stranded DNA (dsDNA) fragments are ligated with adapters, amplified in the presence of a defined concentration of deoxyuracil triphosphate (dUTP) nucleotides, cleaved at the incorporated uracils, and ligated with a sequencing adapter prior to amplification and sequencing. FIG. 3A illustrates that the first and second adapters are, for example, hairpin adapters each including first and second primer binding sequences (indicated as P1 and P2) and a UMI (e.g., UMI1 and UMI2). Although only a single strand of the DNA fragment is shown, it is understood that the hairpin adapters are ligated onto a double-stranded DNA fragment. Following ligation of the adapters, amplification is performed using amplification primers targeting the P1 and P2 primer binding sequences (e.g., performing PCR) in the presence of a defined concentration of dUTP such that, on average, about one uracil is incorporated per extended product (illustrated as a single “U” in the amplification product). In embodiments, alternative cleavable sites and/or cleavable nucleotides may be incorporated into the extended product as described herein. Following extension, the incorporated uracil(s) are cleaved using known chemical and/or enzymatic methods (e.g., with a combination of uracil DNA glycosylase and an abasic site-specific endonuclease), and subsequently end-repaired and A-tailed (not shown). Subsequently, a third adapter including a P3 primer binding sequence is ligated to the cleaved DNA fragment, followed by PCR with primers targeting P2 and P3. The resulting PCR products contain a common UMI that may be used to reconstruct the original sample polynucleotides, as shown in FIG. 1. FIG. 3B illustrates an alternate embodiment for generating adapter-ligated fragments of the template nucleic acid formed in FIG. 3A. PCR with primers specific to the P1/P2 regions is performed, followed by hybridization and extension of P3 sequencing adapter-containing randomer primers (e.g., a primer including a random nucleotide sequence in the hybridization region of the primer). As the randomer primer hybridizes at various different positions of the template nucleic acid (e.g., random hybridization along the template strand), the resulting amplification products will be fragmented at various lengths.

FIGS. 4A-4B illustrate an embodiment utilizing ssDNA to ssDNA ligation for adapter ligation to UMI-containing, fragmented extension products. FIG. 4A illustrates the steps of adapter ligation onto a DNA fragment, wherein the adapter includes a primer binding sequence (e.g., P1) and a UMI (e.g., UMI1). Following adapter ligation, linear amplification by a polymerase (depicted as a cloud object) is performed using a primer with a capture moiety, labeled as CM (e.g., biotin), in the presence of a defined concentration of dUTP nucleotides such that, on average, approximately one uracil is randomly incorporated during extension (incorporated uracils depicted as “U”). As illustrated herein, the linear amplification product is single-stranded. Following amplification, the extension product is pulled down with an affinity substrate (e.g., streptavidin-coated beads, depicted as a sphere), and the incorporated uracils are cleaved (e.g., by uracil DNA glycosylase and abasic site-specific endonuclease cleavage). Subsequently, a single-stranded P2 adapter is ligated onto the cleaved product using 5′ App ligase (wherein the P2 adapter is 5′ adenylated prior to ligation), and subsequent PCR is performed to amplify the ligated product. FIG. 4B illustrates an alternate embodiment for ligating the P2 adapter on to the cleaved product of FIG. 4A. Terminal deoxynucleotidyl transferase (TdT) is used to polyadenylate the 3′ end of the cleaved product, followed by P2 adapter ligation with T4 RNA ligase, and subsequent PCR to amplify the ligated product.

FIG. 5 is an illustration an embodiment of the invention. A polynucleotide including two unique molecular identifiers (UMI1 and UMI2) is fragmented to generate a plurality of fragments of different lengths. Each fragment including at least one of the two UMIs is sequenced, optionally sequencing in both directions (i.e., sequencing the forward and reverse complement of the original fragment) using common paired-read or paired-end sequencing methods, to generate a plurality of variable length sequencing reads, wherein each read contains the fragment and one or both UMIs, or complement thereof. The sequencing reads may then be bioinformatically grouped by the common UMI(s) to reconstruct the original long polynucleotide molecule.

FIGS. 6A-6B illustrate an embodiment of a dual-UMI based amplification approach utilizing an RNA intermediate. FIG. 6A illustrates one embodiment of generating a T7 promoter and UMI-containing adapter for subsequent ligation. The illustrated adapters also include a duplexed constant region (not shown) adjacent to the unique molecular identifier (UMI) sequence. Briefly, a primer is hybridized at the 5′ end of a UMI sequence (e.g., hybridized to the P1 primer binding sequence) and extended using a polymerase with exonuclease activity, such that the UMI sequence is copied, followed by T-tailing (e.g., with Taq polymerase) to leave a T 3′ overhang. FIG. 6B illustrates the steps of adapter ligation to a DNA fragment. Although only a single strand of the DNA fragment is shown, it is understood that in embodiments, the adapters of FIG. 6A may be ligated onto a double-stranded DNA fragment. Following adapter ligation, linear amplification and RNA transcription using RNA polymerase (illustrated as a cloud-shaped object) is performed to generate single-stranded amplification products. Subsequently, a fraction of full-length transcription product is aliquoted and retained (not shown) prior to proceeding to RNA fragmentation. The RNA transcripts are then fragmented (e.g., using a Mg-based fragmentation solution), and a P2-containing adapter is ligated onto each free 3′ end using T4 RNA ligase, and RT-PCR to generate dsDNA (i.e., cDNA) template polynucleotides with distinct UMIs. The retained fraction of full-length RNA transcription products are also ligated with the P2 adapter sequence and reverse transcribed to generate cDNA.

FIGS. 7A-7B illustrate alternate adapters for a dual-UMI based amplification approach utilizing an RNA intermediate. FIG. 7A illustrates one embodiment of generating a T7 promoter and UMI-containing hairpin adapter for subsequent ligation. The illustrated adapters also include a duplexed C1/C2 constant region (alternatively referred to as a stem) adjacent to the UMI sequence. Briefly, the 3′ end of the hairpin adapter is extended using a polymerase with exonuclease activity, such that the UMI sequence is copied, followed by T-tailing to leave a T 3′-overhang. FIG. 7B illustrates a second embodiment for generating a T7 promoter and UMI-containing hairpin adapter for subsequent ligation. Briefly, a 3′-T-tailed oligonucleotide is annealed to the 5′-end of the adapter at an H1 primer binding site, and extension (using a polymerase with exonuclease activity) and ligation (e.g., ligation with T4 DNA ligase) are then performed to copy the UMI sequence and seal the nick between the copied UMI sequence and the annealed oligonucleotide.

FIGS. 8A-8B illustrate an embodiment of a dual-UMI based amplification approach using an RNA intermediate and randomer primers. FIG. 8A illustrates the steps of adapter ligation (e.g., hairpin adapter ligation) onto a dsDNA sample polynucleotide (also referred to herein as a DNA fragment). Although only a single strand of the DNA fragment is shown, it is understood that in embodiments, the hairpin adapters are ligated onto a double-stranded DNA fragment. The adapter includes a duplexed UMI (shown as a single rectangle), a cleavable site (e.g., a uracil), a T7 promoter sequence, and a P1 primer binding site. Following adapter ligation, the hairpin is cleaved (e.g., uracil cleavage by USER enzyme mix) to generate two non-covalently linked strands. Following hairpin cleavage, RNA linear amplification is performed using, for example, T7 RNA polymerase (illustrated as a cloud-shaped object), generating a plurality of single-stranded RNA transcripts. FIG. 8B illustrates the steps of randomer primer hybridization, wherein the randomer primer includes a randomer hybridization region (N's) and a P2 primer binding sequence, to the linear amplification RNA polynucleotide products. Following hybridization, RT-PCR is performed to generate dsDNA (i.e., cDNA) template polynucleotides with distinct UMIs (only one strand of each product is shown).

FIG. 9 illustrates an embodiment of a dual-UMI based amplification approach using an RNA intermediate. Adapter ligation (e.g., hairpin adapter ligation) onto a DNA fragment is performed, wherein each hairpin adapter includes two UMI sequences (e.g., UMI1 and UMI2), a cleavable site (e.g., one or more uracil(s)), a primer binding sequence (e.g., P1) and a duplexed constant region (e.g., C1/C2, shown as a single rectangle) adjacent to the UMIs. Although only a single strand of the DNA fragment is shown, it will be understood that the hairpin adapters are ligated onto a double-stranded DNA fragment. Following adapter ligation, the hairpin adapters are cleaved (e.g., by uracil cleavage) and linear amplification performed using T7 RNA polymerase (illustrated as a cloud-shaped object), generating a plurality of single-stranded RNA transcripts. Following linear amplification, RNA fragmentation (e.g., using a Mg-based fragmentation solution) is performed, and a P2-containing adapter ligated onto the free 3′ ends using T4 RNA ligase. RT-PCR is then performed to generate dsDNA (i.e., cDNA) template polynucleotides with distinct UMIs (not shown).

FIG. 10 illustrates an alternate embodiment of a dual-UMI based amplification approach. First, adapter ligation (e.g., hairpin adapter ligation) on a DNA fragment is performed. Although only a single strand of the DNA fragment is shown, it will be understood that the hairpin adapters are ligated onto a double-stranded DNA fragment. Each hairpin adapter includes two UMI sequences (e.g., UMI1 and UMI2), two primer binding sequences (e.g., P1 and P2), and a duplexed constant region (e.g., C1/C2, shown as a single rectangle). Following adapter ligation, PCR amplification of the template polynucleotide is performed, such that the amplification product includes two UMI sequences. Although only the top strand with UMI1 and UML3 is shown as amplified, it is understood that the bottom strand with UMI2 and UMI4 will also be amplified. Following PCR, a portion of the amplified templates are fragmented (e.g., physical fragmentation), such that some full-length amplified product is retained (not shown). End repair and A-tailing is then performed on both the fragmented and full-length templates (not shown). Next, adapters including platform primer sequences are ligated to both the fragmented and full-length templates. The adapters are shown as hairpin adapters, each including a sequence complementary to a sequencing platform, referred to as S1 and S2. Subsequently, the platform primer-containing ligation products are PCR amplified and sequenced. Although only a single strand is shown following PCR, it is understood that the PCR products may be double-stranded.

FIG. 11 illustrates an embodiment of a rolling circle amplification (RCA)-based approach for generating UMI-containing template polynucleotides for sequencing. Note that while RCA is indicated in the figure, any suitable circular amplification method (e.g., exponential rolling circle amplification (eRCA)) may be used. First, adapter ligation (e.g., hairpin adapter ligation) onto a DNA fragment is performed. Although only a single strand of the DNA fragment is shown, it is understood that in embodiments, the hairpin adapters are ligated onto a double-stranded DNA fragment. Each hairpin adapter includes a duplexed UMI sequence (e.g., UMI1, shown as a single rectangle), two primer binding sequences (e.g., P1 and P2), and a duplexed constant region (e.g., C1/C2, shown as a single rectangle). Rolling circle amplification (or alternatively, eRCA) is then performed using a strand-displacing polymerase (e.g., a phi29 DNA polymerase), followed by fragmentation of the RCA product and end-repair/A-tailing of the fragments. The fragments are then ligated to sequencing adapters (shown as hairpin adapters), wherein the sequencing adapters include a sequencing primer binding sequence (e.g., P3) and a duplexed constant region (e.g., a stem, referred to as C3, shown as a single rectangle). Following sequencing adapter ligation, the samples are then sequenced.

### DETAILED DESCRIPTION

### I. Definitions

Described herein are compositions and methods for mapping sequences, which are especially useful for sequences having large structural variations, e.g., inversions and translocations, tandem repeat regions, distinguishing clinically relevant genes from pseudogenes, and haplotype reconstructions.

The practice of the technology described herein will employ, unless indicated specifically to the contrary, conventional methods of chemistry, biochemistry, organic chemistry, molecular biology, recombinant DNA techniques, genetics, immunology, and cell biology that are within the skill of the art, many of which are described below for the purpose of illustration. Examples of such techniques are available in the literature. Methods, devices, and materials similar or equivalent to those described herein can be used in the practice of this invention.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “promoter” or “promoter sequence” is used in accordance with its plain and ordinary meaning and refers to a sequence of DNA to which RNA polymerases bind to initiate transcription of a single RNA transcript from the DNA downstream of the promoter. The RNA transcript may encode a protein (e.g., mRNA), or can have a function in and of itself, such as tRNA or rRNA. Promoters contain specific DNA sequences such as response elements that provide a secure initial binding site for RNA polymerase. Promoters, for example, may be attached to a double-stranded DNA molecule to enable transcription by an RNA polymerase (see, e.g., Li J and Eberwine J. Nature Protocols. 2018; 13: 811-818, which is incorporated herein by reference in its entirety).

As used herein, the term “consensus sequence” refers to a sequence that shows the nucleotide most commonly found at each position within the nucleic acid sequences of group of sequences (e.g., a group of sequencing reads) aligned at that position. A consensus sequence is often “assembled” from shorter sequence reads that are at least partially overlapping. Where two sequences contain overlapping sequence information aligned at one end and non-overlapping sequence information at opposite ends, the consensus sequence formed from the two sequences will be longer than either sequence individually. Aligning multiple such sequences allows for assembly of many short sequences into much longer consensus sequences representative of a longer sample polynucleotide. In embodiments, aligned sequences used to generate a consensus sequence may contain gaps (e.g., representative of nucleotides not appearing in a given read).

In embodiments, a nucleic acid (e.g., an adapter, linear nucleic acid molecule, or primer) includes a sample barcode. In general, a “sample barcode” is a nucleotide sequence that is sufficiently different from other sample barcodes to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the terms “platform primer” and “platform primer sequence” refer to any polynucleotide sequence including a sequence complementary to a surface-immobilized primer, an optional index sequence for multiplexing samples, and a region complementary to a sequencing primer. One or more platform primer sequences may be used in some embodiments, wherein the platform primer sequence may be included in an adapter sequence (e.g., a P1 or P2 adapter sequence). A first adapter sequence, for example, may include a first platform primer (e.g., pp1), and a second adapter sequence, for example, may include a second platform primer (e.g., pp2). In embodiments, the platform primer sequence is used during amplification reactions (e.g., solid phase amplification). In embodiments, a sequencing primer anneals to the sequencing primer region of the adapter and serves as the initiation point for a sequencing reaction. In embodiments, the platform primer sequence provides complementarity to a sequencing primer.

As used herein, a platform primer is a primer oligonucleotide immobilized or otherwise bound to a solid support (i.e. an immobilized oligonucleotide). Examples of platform primers include P7 and P5 primers, or S1 and S2 sequences, or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

As used herein, the term “loop” is used in accordance with its plain ordinary meaning and refers to the single-stranded region of a hairpin adapter that is located between the duplexed “stem” region of the hairpin adapter. In embodiments, the hairpin loop region is between about 4 nucleotides to 150 nucleotides in length. In embodiments, the hairpin loop is at least 4, 5, 6, 7, 8, 9, 10, 15, 20, 25, 30, 35, 40, 45, 50, or more nucleotides in length. In embodiments, the hairpin loop includes 1, 2, 3, 4, 5, 6, 7, 8, 9, 10, or more T nucleotides. In embodiments, the hairpin loop may include one or more of a primer binding sequence, a barcode, a UMI sequence, or a cleavable site. In some embodiments, a hairpin adapter comprises a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter comprises a stem portion (i.e., stem) and a loop, wherein the stem portion is substantially double stranded thereby forming a duplex. In some embodiments, the loop of a hairpin adapter comprises a nucleic acid strand that is not complementary (e.g., not substantially complementary) to itself or to any other portion of the hairpin adapter.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a particle described herein to interact with an array.

As used herein, the term “random” in the context of a nucleic acid sequence or barcode sequence refers to a sequence where one or more nucleotides has an equal probability of being present. In embodiments, one or more nucleotides is selected at random from a set of two or more different nucleotides at one or more positions, with each of the different nucleotides selected at one or more positions represented in a pool of oligonucleotides including the random sequence. For example, a random sequence may be represented by a sequence composed of N's, where N can be any nucleotide (e.g., A, T, C, or G). For example, a four base random sequence may have the sequence NNNN, where the Ns can independently be any nucleotide (e.g., AATC or GTCA). In embodiments, a pool of barcodes may be represented by a fully random sequence, with the caveat that certain sequences have been excluded (e.g., runs of three or more nucleotides of the same type, such as “AAA” or “GGG”). In embodiments, nucleotide positions that are allowed to vary (e.g., by two, three, or four nucleotides) may be separated by one or more fixed positions (e.g., as in “NGN”).

As may be used herein, the terms “nucleic acid,” “nucleic acid molecule,” “nucleic acid sequence,” “strand,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

As used here, the terms “randomer primer” and “randomer primer oligonucleotide” refer to a synthetic primer including a random sequence. For example, a mixture of randomer primers include a plurality of primers that each have a sequence wherein, during synthesis, each nucleotide has an equal probability of being present. In embodiments, one or more nucleotides of the randomer primer is selected at random from a set of two or more different nucleotides at one or more positions, with each of the different nucleotides selected at one or more positions represented in a pool of oligonucleotides including the random sequence. For example, a randomer primer sequence may be represented by a sequence composed of N's, where N can be any nucleotide (e.g., A, T, C, or G). For example, a six base randomer primer sequence may have the sequence NNNNNN, where the Ns can independently be any nucleotide (e.g., AATCAT or GTCAGA). In embodiments, a pool of randomer primers may be represented by a fully random sequence, with the caveat that certain sequences have been excluded (e.g., runs of three or more nucleotides of the same type, such as “AAA” or “GGG”). In embodiments, nucleotide positions of the randomer primer that are allowed to vary (e.g., by two, three, or four nucleotides) may be separated by one or more fixed positions (e.g., as in “NGN”). For example, a composition including 6-mer randomer primers (i.e., primers including a random sequence of 6 nucleotides) or 9-mer randomer primers (i.e., primers including a random sequence of 9 nucleotides), the composition includes 4⁶ different primer compositions for the 6-mer, and 4⁹ different primer compositions for the 9-mer.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Other analog nucleic acids include bis-locked nucleic acids (bisLNAs; e.g., including those described in Moreno PMD et al. Nucleic Acids Res. 2013; 41(5):3257-73), twisted intercalating nucleic acids (TINAs; e.g., including those described in Doluca O et al. Chembiochem. 2011; 12(15):2365-74), bridged nucleic acids (BNAs; e.g., including those described in Soler-Bistue A et al. Molecules. 2019; 24(12): 2297), 2′-O-methyl RNA:DNA chimeric nucleic acids (e.g., including those described in Wang S and Kool E T. Nucleic Acids Res. 1995; 23(7):1157-1164), minor groove binder (MGB) nucleic acids (e.g., including those described in Kutyavin I V et al. Nucleic Acids Res. 2000; 28(2):655-61), morpholino nucleic acids (e.g., including those described in Summerton J and Weller D. Antisense Nucleic Acid Drug Dev. 1997; 7(3):187-95), C5-modified pyrimidine nucleic acids (e.g., including those described in Kumar P et al. J. Org. Chem. 2014; 79(11): 5047-5061), peptide nucleic acids (PNAs; e.g., including those described in Gupta A et al. J. Biotechnol. 2017; 259: 148-59), and/or phosphorothioate nucleotides (e.g., including those described in Eckstein F. Nucleic Acid Ther. 2014; 24(6):374-87).

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Native nucleotides may include native DNA nucleotides or native RNA nucleotides. Examples of native nucleotides useful for carrying out procedures described herein include: dATP (2′-deoxyadenosine-5′-triphosphate); dGTP (2′-deoxvguanosine-5′-triphosphate); dCTP (2′-deoxycytidine-5′-triphosphate); dTTP (2′-deoxvthymidine-5′-triphosphate); and dUTP (2′-deoxyuridine-5′-triphosphate). Examples of native DNA nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); and dTTP (2′-deoxythymidine-5′-triphosphate). Examples of native RNA nucleotides useful for carrying out procedures described herein include: ATP (adenosine-5′-triphosphate); GTP (guanosine-5′-triphosphate); CTP (cytidine-5′-triphosphate); and UTP (dine-5′-triphosphate).

In embodiments, the nucleotides of the present disclosure use a cleavable linker to attach a label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(O), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S20₄), weak acid, hydrazine (N₂H₄). Pd(O), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels comprising fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, the label is a fluorophore.

In some embodiments, a nucleic acid comprises a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide comprises a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group. e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of reversible terminators are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

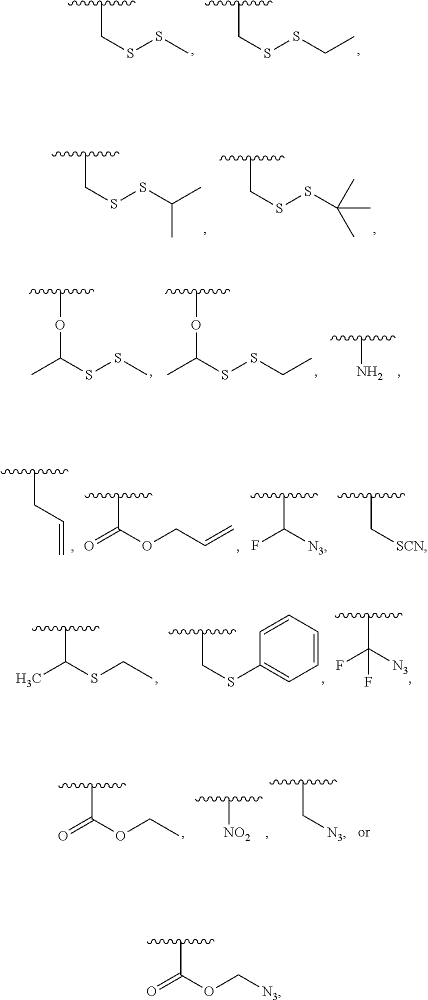

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is

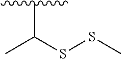 [SMILES: *C(C)SSC]

as described in U.S. Pat. No. 10,738,072, which is incorporated herein by reference for all purposes. For example, a nucleotide including a reversible terminator moiety may be represented by the formula:

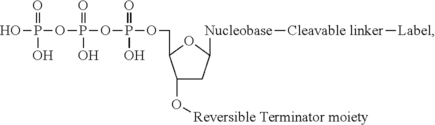

where the nucleobase is adenine or adenine analogue, thymine or thymine analogue, guanine or guanine analogue, or cytosine or cytosine analogue.

In some embodiments, a nucleic acid comprises a molecular identifier or a molecular barcode. As used herein, the term “molecular barcode” (which may be referred to as a “tag”, a “barcode”, a “barcode sequence”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads comprising the barcode can be identified as originating from a single sample polynucleotide molecule on the basis of the barcode alone. In other embodiments, individual barcode sequences may be used more than once, but adapters comprising the duplicate barcodes are associated with different sequences and/or in different combinations of barcoded adapters, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′->3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” or “template nucleic acid” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In the context of selective sequencing, “target polynucleotide(s)” refers to the subset of polynucleotide(s) to be sequenced from within a starting population of polynucleotides.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g. apoptosis or necrosis) or cell shedding, releasing polynucleotides into surrounding body fluids or into circulation. Accordingly, cell-free polynucleotides may be isolated from a non-cellular fraction of blood (e.g. serum or plasma), from other bodily fluids (e.g. urine), or from non-cellular fractions of other types of samples.

As used herein, the terms “specific”, “specifically”, “specificity”, or the like of a compound refers to the compound's ability to cause a particular action, such as binding, to a particular molecular target with minimal or no action to other proteins in the cell.

As used herein, the terms “attached,” “bind,” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g., electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein comprises contacting a template and an annealed primer with a suitable polymerase under conditions suitable for polymerase extension and/or sequencing. In embodiments, sequencing generates one or more sequencing reads. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, silica, diamond, silver, metal, or polypropylene. In embodiments, the solid substrate is porous.

As used herein, the term “consensus sequence” is used in accordance with its plain and ordinary meaning and refers to a theoretical representative nucleotide or amino acid sequence in which each nucleotide or amino acid is the one which occurs most frequently at that site in the different sequences which occur in nature. The phrase also refers to an actual sequence which approximates the theoretical consensus. The consensus sequence is a sequence of DNA, RNA, or protein that represents aligned, related sequences.

As used herein, the term “sequencing reaction mixture” is used in accordance with its plain and ordinary meaning and refers to an aqueous mixture that contains the reagents sufficient to allow a dNTP or dNTP analogue to add a nucleotide to a DNA strand by a DNA polymerase. In embodiments, the sequencing reaction mixture includes a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxvl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), or salts (e.g., ammonium sulfate, magnesium chloride, sodium chloride, or potassium chloride).

As used herein, the terms “solid support” and “substrate” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discemible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures which appear substantially or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further comprise a polymer or hydrogel on the surface to which the primers are attached (e.g., the primers are covalently attached to the polymer, wherein the polymer is in direct contact with the solid support). Exemplary solid supports include, but are not limited to, silica and modified or functionalized silica, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopattemable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface comprising a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate comprises a surface (e.g., a surface of a flow cell, a surface of a tube, a surface of a chip), for example a metal surface (e.g., steel, gold, silver, aluminum, silicon and copper). In some embodiments a substrate (e.g., a substrate surface) is coated and/or comprises functional groups and/or inert materials. In certain embodiments a substrate comprises a bead, a chip, a capillary, a plate, a membrane, a wafer (e.g., silicon wafers), a comb, or a pin for example. In some embodiments a substrate comprises a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, silica, nylon, Wang resin, Merrifield resin, metal (e.g., iron, a metal alloy, sepharose, agarose, polyacrylamide, dextran, cellulose and the like or combinations thereof. In some embodiments a substrate comprises a magnetic material (e.g., iron, nickel, cobalt, platinum, aluminum, and the like). In certain embodiments a substrate comprises a magnetic bead (e.g., DYNABEADS®, hematite, AMPure XP). Magnets can be used to purify and/or capture nucleic acids bound to certain substrates (e.g., substrates comprising a metal or magnetic material).

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer. Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

The term “array” as used herein, refers to a container (e.g., a microplate, tube, or flow cell) including a plurality of features (e.g., wells, microwells, nanowells). For example, an array may include a container with a plurality of wells. In embodiments, the array is a microplate. In embodiments, the array is a flow cell.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. The sequence reads are optionally stored in an appropriate data structure for further evaluation. In embodiments, a first sequencing reaction can generate a first sequencing read. The first sequencing read can provide the sequence of a first region of the polynucleotide fragment. In embodiments, a second sequencing primer can initiate sequencing at a second location on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some embodiments, the nucleic acid template is optionally subjected to one or more additional rounds of sequencing using additional sequencing primers, thereby generating additional sequencing reads.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often comprise nucleic acid sequences that are substantially complementary to each other.

“Hybridize” shall mean the annealing of one single-stranded nucleic acid sequence (such as a primer) to another nucleic acid sequence based on the well-understood principle of sequence complementarity. In an embodiment the other nucleic acid sequence is a single-stranded nucleic acid. The propensity for hybridization between nucleic acid sequences depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. In some embodiments, nucleic acids, or portions thereof, that are configured to hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which comprises a double-stranded portion of nucleic acid.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which comprises a double stranded portion of nucleic acid.

As used herein, “hybridizing” or “annealing” are used interchangeably in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the melting temperature (Tm) of the formed hybrid, and the G:C ratio within the nucleic acids. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. For example, hybridizing a primer to a polynucleotide strand includes combining the primer and the polynucleotide strand in a reaction vessel under suitable hybridization reaction conditions.

As used herein, “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, “capable of hybridizing” is used in accordance with its ordinary meaning in the art and refers to two oligonucleotides that, under suitable conditions, can form a duplex (e.g., Watson-Crick pairing) which includes a double-stranded portion of nucleic acid. Such conditions, known in the art and described herein, depend upon, for example, the nature of the nucleotide sequence, temperature, and buffer conditions. The stringency of hybridization can be influenced by various parameters, including degree of identity and/or complementarity between the polynucleotides (or any target sequences within the polynucleotides) to be hybridized; melting point of the polynucleotides and/or target sequences to be hybridized, referred to as “Tm”; parameters such as salts, buffers, pH, temperature, GC % content of the polynucleotide and primers, and/or time. Typically, hybridization is favored in lower temperatures and/or increased salt concentrations, as well as reduced concentrations of organic solvents. Some exemplary conditions suitable for hybridization include incubation of the polynucleotides to be hybridized in solutions having sodium salts, such as NaCl, sodium citrate and/or sodium phosphate. In some embodiments, hybridization or wash solutions can include about 10-75% formamide and/or about 0.01-0.7% sodium dodecyl sulfate (SDS). In some embodiments, a hybridization solution can be a stringent hybridization solution which can include any combination of 50% formamide, 5×SSC (0.75 M NaCl, 0.075 M sodium citrate), 50 mM sodium phosphate (pH 6.8), 0.1% sodium pyrophosphate, 5×Denhardt's solution, 0.1% SDS, and/or 10% dextran sulfate. In some embodiments, the hybridization or washing solution can include BSA (bovine serum albumin). In some embodiments, hybridization or washing can be conducted at a temperature range of about 20-25° C., or about 25-30° C., or about 30-35° C., or about 35-40° C., or about 40-45° C., or about 45-50° C., or about 50-55° C., or higher. In some embodiments, hybridization or washing can be conducted for a time range of about 1-10 minutes, or about 10-20 minutes, or about 20-30 minutes, or about 30-40 minutes, or about 40-50 minutes, or about 50-60 minutes, or longer. In some embodiments, hybridization or wash conditions can be conducted at a pH range of about 5-10, or about pH 6-9, or about pH 6.5-8, or about pH 6.5-7.

As used herein, the term “non-targeted template hybridization” refers to the pairing of complementary nucleic acids using any process by which a strand of nucleic acid joins with a complementary strand through base pairing to form a hybridization complex, wherein the strand of nucleic acid and/or the complementary strand include one or more degenerate bases (also referred to herein as random bases). For example, non-targeted template hybridization of a “randomer primer”, as used herein, refers to a primer including a plurality of degenerate bases at nucleotide positions that hybridize to a complementary polynucleotide strand. The degenerative portion of the encoded sequence (e.g., of a randomer primer) is incorporated by mixing the DNA phosphoramidites during the synthetic procedure and generating variants of the corresponding combinations of A, C, G and T. Additional nucleoside analogs, known as universal bases, which pair with native bases, may also be used to generate a degenerate sequence for non-targeted template hybridization. Examples of universal bases include 3-nitropyrrole and 5-nitroindole, and are discussed further, e.g., in Loakes D. Nucleic Acids Res. 2001; 29(12):2437-47.

As used herein, the terms “denaturant” or plural “denaturants” are used in accordance with their plain and ordinary meanings and refer to an additive or condition that disrupts the base pairing between nucleotides within opposing strands of a double-stranded polynucleotide molecule. The term “denature” and its variants, when used in reference to any double-stranded polynucleotide molecule, or double-stranded polynucleotide sequence, includes any process whereby the base pairing between nucleotides within opposing strands of the double-stranded molecule, or double-stranded sequence, is disrupted. Typically, denaturation includes rendering at least some portion or region of two strands of the double-stranded polynucleotide molecule or sequence single-stranded or partially single-stranded. In some embodiments, denaturation includes separation of at least some portion or region of two strands of the double-stranded polynucleotide molecule or sequence from each other. Typically, the denatured region or portion is then capable of hybridizing to another polynucleotide molecule or sequence. Optionally, there can be “complete” or “total” denaturation of a double-stranded polynucleotide molecule or sequence. Complete denaturation conditions are, for example, conditions that would result in complete separation of a significant fraction (e.g., more than 10%, 20%, 30%, 40% or 50%) of a large plurality of strands from their extended and/or full-length complements. Typically, complete or total denaturation disrupts all of the base pairing between the nucleotides of the two strands with each other. Similarly, a nucleic acid sample is optionally considered fully denatured when more than 80% or 90% of individual molecules of the sample lack any double-strandedness (or lack any hybridization to a complementary strand).

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction comprises a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that comprises a polymerase chain reaction (PCR). Conditions conducive to amplification (i.e., amplification conditions) are well known and often comprise at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction-CCR), and the like.

Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18—(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers comprising tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyperbranched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification comprises a nucleic acid amplification reaction comprising only one species of oligonucleotide primer immobilized to a surface or substrate. In certain embodiments solid phase amplification comprises a plurality of different immobilized oligonucleotide primer species. In some embodiments solid phase amplification may comprise a nucleic acid amplification reaction comprising one species of oligonucleotide primer immobilized on a solid surface and a second different oligonucleotide primer species in solution. Multiple different species of immobilized or solution-based primers can be used.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods and compositions for analyzing a sample (e.g., sequencing nucleic acids within a sample). A sample (e.g., a sample comprising nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may comprise cells or tissues that are normal, healthy, diseased (e.g., infected), and/or cancerous (e.g., cancer cells). A sample obtained from a subject may comprise cells or cellular material (e.g., nucleic acids) of multiple organisms (e.g., virus nucleic acid, fetal nucleic acid, bacterial nucleic acid, parasite nucleic acid).

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system comprising two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate reactive group 1 | Bioconjugate reactive group 2 | |
|---|---|---|
| (e.g., electrophilic bioconjugate | (e.g., nucleophilic bioconjugate | Resulting Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine). Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or strepavidin to form a avidin-biotin complex or streptavidin-biotin complex.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

The term “adapter” as used herein refers to any linear oligonucleotide that can be ligated to a nucleic acid molecule, thereby generating nucleic acid products that can be sequenced on a sequencing platform (e.g., an Illumina or Singular Genomics G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing.

As used herein, the term “hairpin adapter” refers to a polynucleotide including a double-stranded stem portion and a single-stranded hairpin loop portion. In some embodiments, an adapter is a hairpin adapter (also referred to herein as a “hairpin”). In some embodiments, a hairpin adapter includes a single nucleic acid strand including a stem-loop structure. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a method herein includes ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

“Synthetic” agents refer to non-naturally occurring agents, such as enzymes or nucleotides.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

“GC bias” describes the relationship between GC content and read coverage across a genome. For example, a genomic region of a higher GC content tends to have more (or less) sequencing reads covering that region. As described herein, GC bias can be introduced during amplification of library, cluster amplification, and/or the sequencing reactions.

By aqueous solution herein is meant a liquid comprising at least 20 vol % water. In embodiments, aqueous solution includes at least 50%, for example at least 75 vol %, at least 95 vol %, above 98 vol %, or 100 vol % of water as the continuous phase.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics Systems™, Inc. (e.g., the G4™ system), Illumina™, Inc. (e.g., HiSeq™, MiSeq™ NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Methods

In an aspect is provided a method of sequencing a polynucleotide, the method including: contacting the polynucleotide including a first unique molecular identifier (UMI) sequence and a promoter sequence with an RNA polymerase and generating a plurality of RNA molecules, wherein each RNA molecule includes a complement of the first UMI; fragmenting the plurality of RNA molecules to form a population of RNA nucleic acid fragments; attaching the population of RNA nucleic acid fragments to a solid support thereby forming a plurality of immobilized RNA nucleic acid fragments, and amplifying the plurality of immobilized RNA nucleic acid fragments to form amplification products immobilized to the solid support; hybridizing a sequencing primer to one or more of the amplification products and incorporating one or more nucleotides into the sequencing primer with a polymerase thereby forming one or more incorporated nucleotides; and detecting the one or more incorporated nucleotides thereby generating a sequencing read.

In embodiments, the method further includes attaching an adapter including a second UMI to the RNA nucleic acid fragments. In embodiments, attaching the adapter includes ligating the adapter with a ligase (e.g., T4 RNA ligase). In embodiments, the method further includes sequencing the first UMI sequence and the second UMI sequence, thereby generating a plurality of sequencing reads, and grouping the plurality of sequencing reads based on co-occurrence of each of the UMI sequences. In embodiments, grouping the plurality of sequencing reads is performed by a computer, wherein the computer groups the plurality of sequencing reads based on co-occurrence of each of the UMI sequences, and outputs the results.

In embodiments, fragmenting the plurality of RNA molecules includes contacting the plurality of RNA molecules with a plurality of oligonucleotide primers, and extending the plurality of oligonucleotide primers, wherein each oligonucleotide primer includes a random sequence and a platform primer binding sequence. In embodiments, each oligonucleotide primer includes, from 5′ to 3′, the platform primer binding sequence and the random sequence.

In embodiments, the random sequence is about 4 to about 30 nucleotides in length. In embodiments, the random sequence is about 6 to about 26 nucleotides in length. In embodiments, the random sequence is about 8 to about 24 nucleotides in length. In embodiments, the random sequence is about 4, 8, 12, 16, 20, 24, 28, or 30 nucleotides in length.

In embodiments, the method includes attaching an adapter including a primer binding sequence to each of the RNA nucleic acid fragments. In embodiments, the method includes a primer binding sequence to each of the RNA nucleic acid fragments. In embodiments, the method ligating a primer binding sequence to each of the RNA nucleic acid fragments.

In embodiments, amplifying includes hybridizing an immobilized DNA oligonucleotide to the plurality of RNA nucleic acid fragments and extending the immobilized DNA oligonucleotide with a reverse transcriptase to form cDNA amplification products immobilized to the solid support.

In embodiments, prior to attaching the population of RNA nucleic acid fragments to a solid support, the method further includes amplifying the population of RNA nucleic acid fragments to generate a population of DNA nucleic acid fragments. In embodiments, the method further includes hybridizing an immobilized DNA oligonucleotide to the DNA nucleic acid fragments and extending the immobilized DNA oligonucleotide with a polymerase to form amplification products immobilized to the solid support.

In embodiments, the method further includes, prior to fragmenting, attaching a primer binding sequence to a full-length RNA molecule, amplifying the full-length RNA molecule to form full-length DNA molecules, and attaching the RNA nucleic acid fragments and full-length DNA molecules to the solid support. In embodiments, the method further includes sequencing the full-length DNA molecules.

In embodiments, the population of RNA nucleic acid fragments includes a collection of fragments having an average length of about 200 nucleotides, plus or minus 100 nucleotides. In embodiments, the population of RNA nucleic acid fragments includes a collection of fragments having an average length of about 200 nucleotides, plus or minus 75 nucleotides. In embodiments, the population of RNA nucleic acid fragments includes a collection of fragments having an average length of about 200 nucleotides, plus or minus 50 nucleotides. In embodiments, the population of RNA nucleic acid fragments includes a collection of fragments having an average length of about 200 nucleotides, plus or minus 25 nucleotides. In embodiments, the population of RNA nucleic acid fragments includes a collection of fragments having an average length of about 300 nucleotides, plus or minus 100 nucleotides. In embodiments, the population of RNA nucleic acid fragments includes a collection of fragments having an average length of about 300 nucleotides, plus or minus 75 nucleotides. In embodiments, the population of RNA nucleic acid fragments includes a collection of fragments having an average length of about 300 nucleotides, plus or minus 50 nucleotides. In embodiments, the population of RNA nucleic acid fragments includes a collection of fragments having an average length of about 300 nucleotides, plus or minus 25 nucleotides. In embodiments, the population of RNA nucleic acid fragments includes a collection of fragments having an average length of about 400 nucleotides, plus or minus 100 nucleotides. In embodiments, the population of RNA nucleic acid fragments includes a collection of fragments having an average length of about 400 nucleotides, plus or minus 75 nucleotides. In embodiments, the population of RNA nucleic acid fragments includes a collection of fragments having an average length of about 400 nucleotides, plus or minus 50 nucleotides. In embodiments, the population of RNA nucleic acid fragments includes a collection of fragments having an average length of about 400 nucleotides, plus or minus 25 nucleotides.

In embodiments, the population of RNA nucleic acid fragments includes polynucleotides from about 30 to about 500 nucleotides in length. In embodiments, the population of RNA nucleic acid fragments includes polynucleotides from about 75 to about 400 nucleotides in length. In embodiments, the population of RNA nucleic acid fragments includes polynucleotides from about 100 to about 300 nucleotides in length. In embodiments, the population of RNA nucleic acid fragments includes polynucleotides from about 150 to about 250 nucleotides in length. In embodiments, the population of RNA nucleic acid fragments includes polynucleotides of at least about 30 nucleotides in length. In embodiments, the population of RNA nucleic acid fragments includes polynucleotides of at least about 50 nucleotides in length. In embodiments, the population of RNA nucleic acid fragments includes polynucleotides of at least about 75 nucleotides in length. In embodiments, the population of RNA nucleic acid fragments includes polynucleotides of at least about 100 nucleotides in length. In embodiments, the population of RNA nucleic acid fragments includes polynucleotides of at least about 200 nucleotides in length. In embodiments, the population of RNA nucleic acid fragments includes polynucleotides of at least about 300 nucleotides in length.

In an aspect is provided a method of sequencing a sample polynucleotide including a first primer binding sequence. In embodiments, the method includes: a) hybridizing a primer to the first primer binding sequence and extending the primer to form an extension strand, wherein extending includes incorporating one or more cleavable sites into the extension strand; b) cleaving the one or more cleavable sites to generate a nucleic acid fragment including a 3′ end; c) ligating an adapter to the 3′ end of the nucleic acid fragment, wherein the adapter includes a sequencing primer binding sequence; d) hybridizing a sequencing primer to the sequencing primer binding sequence and incorporating one or more nucleotides into the sequencing primer with a polymerase; and detecting the one or more incorporated nucleotides thereby sequencing the sample polynucleotide.

In an aspect is provided a method of sequencing a sample polynucleotide including a promoter sequence. In embodiments, the method includes: a) hybridizing a a primer complementary to the promoter sequence, to the promoter sequence and transcribing the sample polynucleotide with an RNA polymerase to generate an RNA amplification product; b) annealing two or more DNA oligonucleotides to the RNA amplification product and extending the hybridized DNA oligonucleotides with a reverse transcriptase to generate a plurality of cDNA products, wherein each DNA oligonucleotide includes a platform primer binding sequence; c) hybridizing a sequencing primer to a cDNA product and incorporating one or more nucleotides into the sequencing primer; and d) detecting the one or more incorporated nucleotides, thereby sequencing the sample polynucleotide.

In an aspect is provided a method of sequencing a sample polynucleotide including a promoter sequence, the method including: a) contacting the sample polynucleotide with a composition including a plurality ofnucleotides and an RNA polymerase thereby forming a plurality of amplification products; b) contacting the sample polynucleotide with a composition including a plurality of randomer primer oligonucleotides and extending the randomer primer oligonucleotides with a reverse transcriptase to form a population of different-sized nucleic acid fragments, wherein each of the randomer primer oligonucleotides includes a platform primer binding sequence; c) binding the nucleic acid fragments to an immobilized primer on a solid support, and amplifying the nucleic acid fragments to form colonies of immobilized polynucleotide fragments, wherein amplifying includes a plurality of cycles of primer extension, denaturation, and primer hybridization; and d) hybridizing a sequencing primer to one or more of the immobilized polynucleotide fragments within the colonies and incorporating one or more nucleotides into the sequencing primer with a polymerase; and detecting the one or more incorporated nucleotides thereby sequencing the sample polynucleotide.

In embodiments, the method includes hybridizing a sequencing primer to a cDNA product and incorporating one or more nucleotides into the sequencing primer with a polymerase to create an extension strand. In embodiments, the method includes detecting the one or more incorporated nucleotides so as to identify each incorporated nucleotide in the extension strand, thereby sequencing the sample polynucleotide.

In embodiments, prior to step c) contacting the RNA amplification product with an extension primer and extending to generate a complementary strand. In embodiments, the extension primer hybridizes to the promoter sequence.

In an aspect is provided a method of sequencing a sample polynucleotide, the method including: a) contacting the sample polynucleotide with a composition and a polymerase, wherein the composition includes a plurality of native DNA nucleotides and cleavable site nucleotides, thereby forming a plurality of amplification products, wherein the amplification products include a cleavable site nucleotide at a different position relative to each other; b) cleaving the amplification products at the cleavable site nucleotide to form a population of different-sized nucleic acid fragments including a 3′ end; c) ligating an adapter to the 3′ end of each of the population of different-sized nucleic acid fragments thereby forming adapter fragments, wherein the adapter includes a sequencing primer binding sequence; d) binding the adapter fragments to immobilized primers on a solid support, and amplifying the adapter fragments to form colonies of immobilized polynucleotide fragments, wherein the amplifying includes a plurality of cycles of primer extension, denaturation, and primer hybridization; and e) hybridizing a sequencing primer to one or more of the immobilized polynucleotide fragments within the colonies and incorporating one or more nucleotides into the sequencing primer with a polymerase; and detecting the one or more incorporated nucleotides thereby sequencing the sample polynucleotide. In embodiments, prior to step d), the adapter fragments are not amplified in solution. In embodiments, the sample polynucleotide includes a promoter sequence.

In embodiments, step a) includes contacting the sample polynucleotide with a composition including a plurality of nucleotides and a primer complementary to the promoter sequence, and transcribing the sample polynucleotide with an RNA polymerase thereby forming a plurality of amplification products.

In an aspect is provided a method of sequencing a polynucleotide, the method including: a) contacting the polynucleotide with an amplification reagent and generating a first complement of the polynucleotide including an incorporated first cleavable site nucleotide at a first position; contacting the polynucleotide with the amplification reagent and generating a second complement of the polynucleotide including a second incorporated cleavable site nucleotide at a second position, wherein the first position and second position are different; wherein the amplification reagent includes a polymerase, a plurality of native DNA nucleotides, and a plurality of cleavable site nucleotides; b) cleaving the first complement at the first position and cleaving the second complement at the second position to form nucleic acid fragments including a 3′ end; c) ligating an adapter to the 3′ end of each of the nucleic acid fragments thereby forming adapter fragments, wherein the adapter includes a sequencing primer binding sequence; d) attaching the adapter fragments to immobilized primers on a solid support, and amplifying the adapter fragments to form amplification products immobilized to the solid support; and e) sequencing the amplification products, or complements thereof.

In embodiments, the plurality of native DNA nucleotides includes a plurality of dATP nucleotides, a plurality of dCTP nucleotides, a plurality of dTTP nucleotides, and a plurality of dGTP nucleotides. In embodiments, the plurality of native DNA nucleotides does not include modified nucleotides.

In embodiments, the cleavable site nucleotide is a deoxy uracil triphosphate (dUTP), a deoxy-8-oxo-guanine triphosphate (d-8-oxoG), a methylated nucleotide, or a nbonucleotide.

In embodiments, the polynucleotide includes a first adapter and a second adapter, wherein the first adapter is a Y-adapter, a hairpin adapter, a blunt-ended adapter, or an adapter including a single-strand overhang and the second adapter is a Y-adapter, a hairpin adapter, a blunt-ended adapter, or an adapter including a single-strand overhang. In embodiments, the first adapter, the second adapter, or both the first adapter and the second adapter include a UMI sequence. In embodiments, each adapter includes, from 5′ to 3′, a UMI sequence, a primer binding site, and a promoter sequence. In embodiments, each adapter includes (i) a first strand including, from 5′ to 3′, a UMI sequence, a first primer binding sequence, a second primer binding sequence, and a promoter sequence; and (ii) a second strand including, from 3′ to 5′, a sequence complementary to the UMI sequence, and a sequence complementary to the first primer binding sequence. In embodiments, each adapter includes, from 5′ to 3′, a UMI sequence, a primer binding sequence, a promoter sequence, a cleavable site, and a sequence complementary to the UMI sequence. In embodiments, each adapter includes, from 5′ to 3′, a first UMI sequence, a primer binding site, a promoter sequence, and a second UMI sequence. In embodiments, each adapter includes a cleavable site.

In embodiments, the polynucleotide includes a promoter sequence. In embodiments, the amplification reagent includes a primer complementary to the promoter sequence, and wherein the polymerase is an RNA polymerase and step a) includes transcribing the polynucleotide with the RNA polymerase thereby forming a plurality of RNA amplification products. In embodiments, the promoter sequence is a T3 RNA polymerase promoter sequence, T5 RNA polymerase promoter sequence, or T7 RNA polymerase promoter sequence. In embodiments, the method further includes, prior to step b), fragmenting the plurality of RNA amplification products to generate a plurality of RNA nucleic acid fragments, wherein the plurality of RNA nucleic acid fragments are include a 3′ end, and ligating the adapter sequence to the 3′ end of each of the plurality of RNA nucleic acid fragments. In embodiments, the adapter includes single-stranded RNA. In embodiments, the adapter sequence is ligated onto a single-stranded nucleic acid with a ligase, wherein the ligase is T4 RNA ligase.

In an aspect is provided a method of forming amplification products and sequencing the amplification products. In embodiments, the method includes attaching a first unique molecular identifier (UMI) to a polynucleotide, fragmenting the polynucleotide to form a plurality of amplification products including the first UMI. In embodiments, the method includes attaching a second UMI to one or more of the amplification products. In embodiments, the method includes sequencing the plurality of amplification products.

In embodiments, the population of different-sized nucleic acid fragments includes a collection of fragments having an average length of about 200 nucleotides, plus or minus 100 nucleotides. In embodiments, the population of different-sized nucleic acid fragments includes a collection of fragments having an average length of about 200 nucleotides, plus or minus 75 nucleotides. In embodiments, the population of different-sized nucleic acid fragments includes a collection of fragments having an average length of about 200 nucleotides, plus or minus 50 nucleotides. In embodiments, the population of different-sized nucleic acid fragments includes a collection of fragments having an average length of about 200 nucleotides, plus or minus 25 nucleotides. In embodiments, the population of different-sized nucleic acid fragments includes a collection of fragments having an average length of about 300 nucleotides, plus or minus 100 nucleotides. In embodiments, the population of different-sized nucleic acid fragments includes a collection of fragments having an average length of about 300 nucleotides, plus or minus 75 nucleotides. In embodiments, the population of different-sized nucleic acid fragments includes a collection of fragments having an average length of about 300 nucleotides, plus or minus 50 nucleotides. In embodiments, the population of different-sized nucleic acid fragments includes a collection of fragments having an average length of about 300 nucleotides, plus or minus 25 nucleotides. In embodiments, the population of different-sized nucleic acid fragments includes a collection of fragments having an average length of about 400 nucleotides, plus or minus 100 nucleotides. In embodiments, the population of different-sized nucleic acid fragments includes a collection of fragments having an average length of about 400 nucleotides, plus or minus 75 nucleotides. In embodiments, the population of different-sized nucleic acid fragments includes a collection of fragments having an average length of about 400 nucleotides, plus or minus 50 nucleotides. In embodiments, the population of different-sized nucleic acid fragments includes a collection of fragments having an average length of about 400 nucleotides, plus or minus 25 nucleotides.

In embodiments, the population of different-sized nucleic acid fragments includes polynucleotides from about 30 to about 500 nucleotides in length. In embodiments, the population of different-sized nucleic acid fragments includes polynucleotides from about 75 to about 400 nucleotides in length. In embodiments, the population of different-sized nucleic acid fragments includes polynucleotides from about 100 to about 300 nucleotides in length. In embodiments, the population of different-sized nucleic acid fragments includes polynucleotides from about 150 to about 250 nucleotides in length. In embodiments, the population of different-sized nucleic acid fragments includes polynucleotides of at least about 30 nucleotides in length. In embodiments, the population of different-sized nucleic acid fragments includes polynucleotides of at least about 50 nucleotides in length. In embodiments, the population of different-sized nucleic acid fragments includes polynucleotides of at least about 75 nucleotides in length. In embodiments, the population of different-sized nucleic acid fragments includes polynucleotides of at least about 100 nucleotides in length. In embodiments, the population of different-sized nucleic acid fragments includes polynucleotides of at least about 200 nucleotides in length. In embodiments, the population of different-sized nucleic acid fragments includes polynucleotides of at least about 300 nucleotides in length.

In embodiments, the immobilized primers are attached to the solid support at their 5′ ends. In embodiments, the immobilized primers attached to the solid support via a linker. The linker may also include spacer nucleotides. Including spacer nucleotides in the linker puts the polynucleotide in an environment having a greater resemblance to free solution. This can be beneficial, for example, in enzyme-mediated reactions such as sequencing-by-synthesis. It is believed that such reactions suffer less steric hindrance issues that can occur when the polynucleotide is directly attached to the solid support or is attached through a very short linker (e.g., a linker including about 1 to 3 carbon atoms). Spacer nucleotides form part of the polynucleotide but do not participate in any reaction carried out on or with the polynucleotide (e.g. a hybridization or amplification reaction). In embodiments, the spacer nucleotides include 1 to 20 nucleotides. In embodiments, the linker includes 10 spacer nucleotides. In embodiments, the linker includes 12 spacer nucleotides. In embodiments, the linker includes 15 spacer nucleotides. It is preferred to use polyT spacer nucleotides, although other nucleotides and combinations thereof can be used. In embodiments, the linker includes 10, 11, 12, 13, 14, or 15 dT spacer nucleotides. In embodiments, the linker includes 12 dT spacer nucleotides. Spacer nucleotides are typically included at the 5′ ends of polynucleotides which are attached to a suitable support. Attachment can be achieved via a phosphorothioate present at the 5′ end of the polynucleotide, an azide moiety, a dibenzocyclooctyne (DBCO) moiety, or any other bioconjugate reactive moiety. The linker may be a carbon-containing chain such as those of formula —(CH₂)n- wherein “n” is from 1 to about 1000. However, a variety of other linkers may be used so long as the linkers are stable under conditions used in DNA sequencing. In embodiments, the linker includes polyethylene glycol (PEG) having a general formula of —(CH₂—CH₂—O)m-, wherein m is from about 1 to 500. In embodiments, m is 8 to 24. In embodiments, m is 10 to 12. In embodiments, the linker, or the immobilized oligonucleotides (e.g., primers) include a cleavable site. In embodiments, a cleavable site is a location which allows controlled cleavage of the immobilized polynucleotide strand (e.g., the linker, the primer, or the polynucleotide) by chemical, enzymatic or photochemical means. In embodiments, the cleavable site includes one or more deoxyuracil nucleobases (dUTPs).

In embodiments, the immobilized primers are covalently attached to the solid support. In embodiments, the 5′ end of the immobilized primers contains a reacted functional group that served to tether the immobilized primers to the solid support (e.g., a bioconjugate linker). Non-limiting examples of covalent attachment include amine-modified polynucleotides reacting with epoxy or isothiocyanate groups on the solid support, succinylated polynucleotides reacting with aminophenyl or aminopropyl functional groups on the solid support, dibenzocycloctyne-modified polynucleotides reacting with azide functional groups on the solid support (or vice versa), trans-cyclooctyne-modified polynucleotides reacting with tetrazine or methyl tetrazine groups on the solid support (or vice versa), disulfide modified polynucleotides reacting with mercapto-functional groups on the solid support, amine-functionalized polynucleotides reacting with carboxylic acid groups via 1-ethyl-3-(3-dimethylaminopropyl)-carbodiimide hydrochloride (EDC) chemistry, thiol-modified polynucleotides attaching to a solid support via a disulfide bond or maleimide linkage, alkyne-modified polynucleotides attaching to a solid support via copper-catalyzed click reactions to azide functional groups on the solid support, and acrydite-modified polynucleotides polymerizing with free acrylic acid monomers on the solid support to form polyacrylamide or reacting with thiol groups on the solid support. In embodiments, the primer is attached to the solid support polymer through electrostatic binding. For example, the negatively charged phosphate backbone of the primer may be bound electrostatically to positively charged monomers in the solid support.

In embodiments, each of the plurality of immobilized oligonucleotides (e.g., immobilized primers) is about 5 to about 25 nucleotides in length. In embodiments, each of the plurality of immobilized oligonucleotides (e.g., immobilized primers) is about 10 to about 40 nucleotides in length. In embodiments, each of the plurality of immobilized oligonucleotides (e.g., immobilized primers) is about 5 to about 100 nucleotides in length. In embodiments, each of the plurality of immobilized oligonucleotides (e.g., immobilized primers) is about 20 to 200 nucleotides in length. In embodiments, each of the plurality of immobilized oligonucleotides (e.g., immobilized primers) about or at least about 5, 6, 7, 8, 9, 10, 12, 15, 18, 20, 25, 30, 35, 40, 50 or more nucleotides in length.

In embodiments, the immobilized oligonucleotides include one or more phosphorothioate nucleotides. In embodiments, the immobilized oligonucleotides include a plurality of phosphorothioate nucleotides. In embodiments, about or at least about 20%, 30%, 40%, 50%, 60%, 70%, 80%, 90%, or about 100% of the nucleotides in the immobilized oligonucleotides are phosphorothioate nucleotides. In embodiments, most of the nucleotides in the immobilized oligonucleotides are phosphorothioate nucleotides. In embodiments, all of the nucleotides in the immobilized oligonucleotides are phosphorothioate nucleotides. In embodiments, none of the nucleotides in the immobilized oligonucleotides are phosphorothioate nucleotides. In embodiments, the 5′ end of the immobilized oligonucleotide includes one or more phosphorothioate nucleotides. In embodiments, the 5′ end of the immobilized oligonucleotide includes between one and five phosphorothioate nucleotides.

In embodiments, the immobilized primers may be referred to as amplification primers. In embodiments, the amplification primers are each attached to the solid support (i.e., immobilized on the surface of a solid support). The polynucleotide molecules can be fixed to surface by a variety of techniques, including covalent attachment and non-covalent attachment. In embodiments, the polynucleotides are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have polynucleotides that exceeds the amount or concentration present at the interstitial regions. In some embodiments the polynucleotides and/or primers may not be present at the interstitial regions. In embodiments, at least two different primers are attached to the solid support (e.g., a forward and a reverse primer), which facilitates generating multiple amplification products from the first extension product or a complement thereof.

In embodiments, the amplification products are localized to sites (e.g., wells) on a solid support, which may be referred to as clusters following generation of a plurality of immobilized amplification products. In embodiments, the clusters have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μm or a number or a range between any two of these values. In embodiments, the mean or median separation is about 0.1-10 microns. In embodiments, the mean or median separation is about 0.25-5 microns. In embodiments, the mean or median separation is about 0.5-2 microns. In embodiments, the mean or median separation is about or at least about 0.1 μm. In embodiments, the mean or median separation is about or at least about 0.25 μm. In embodiments, the mean or median separation is about or at least about 0.5 μm. In embodiments, the mean or median separation is about or at least about 1.0 μm. In embodiments, the mean or median separation is about or at least about 1.5 μm. In embodiments, the mean or median separation is about or at least about 2.0 μm. In embodiments, the mean or median separation is about or at least about 5.0 μm. In embodiments, the mean or median separation is about or at least about 10 μm. The mean or median separation may be measured center-to-center (i.e., the center of one cluster to the center of a second cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm. In embodiments, the mean or median separation is about or at least about 1.0, 1.1, 1.2, 1.3, 1.4, 1.5, 1.6, 1.7, 1.8, 1.9, or 2.0 μm. In embodiments, the mean or median separation is about 1.0, 1.1, 1.2, 1.3, 1.4, 1.5, 1.6, 1.7, 1.8, 1.9, or 2.0 μm.

In embodiments, the method includes contacting the sample polynucleotide with a composition including a plurality of primer oligonucleotides, wherein the primer oligonucleotides each include a random sequence (e.g., a randomly synthesized 6-9 nucleotide sequence). In embodiments, the composition includes a plurality of native DNA nucleotides including a plurality of dATP (2′-deoxyadenosine-5′-triphosphate) nucleotides, dCTP (2′-deoxycytidine-5′-triphosphate) nucleotides, dTTP (2′-deoxythymidine-5′-triphosphate) nucleotides, and dGTP (2′-deoxyguanosine-5′-triphosphate) nucleotides. In embodiments, the composition includes a plurality of dATP (2′-deoxyadenosine-5′-triphosphate) nucleotides, dCTP (2′-deoxycytidine-5′-triphosphate) nucleotides, dTTP (2′-deoxythymidine-5′-triphosphate) nucleotides, and dGTP (2′-deoxyguanosine-5′-triphosphate) nucleotides. In embodiments, the composition includes a plurality of native DNA nucleotides including a plurality of dATP nucleotides, dCTP nucleotides, dTTP nucleotides, or dGTP nucleotides. In embodiments, the composition includes a plurality of dATP nucleotides, dCTP nucleotides, dTTP nucleotides, or dGTP nucleotides. In embodiments, the composition includes a plurality of dATP nucleotides. In embodiments, the composition includes a plurality of dCTP nucleotides. In embodiments, the composition includes a plurality of dTTP nucleotides. In embodiments, the composition includes a plurality of dGTP nucleotides. In embodiments, the composition includes a plurality of dUTP (2′-deoxycytidine-5′-triphosphate) nucleotides. In embodiments, the composition consists of a plurality of dA nucleotides, a plurality of dC nucleotides, a plurality of dT nucleotides, and a plurality of dG nucleotides. In embodiments, the composition consists of a plurality of dA nucleotides, a plurality of dC nucleotides, a plurality of dT nucleotides, a plurality of dU nucleotides, and a plurality of dG nucleotides.

In embodiments, the composition includes a plurality of native RNA nucleotides (i.e., native ribonucleotides) including a plurality of ATP (adenosine-5′-triphosphate) nucleotides, CTP (cytidine-5′-triphosphate) nucleotides, UTP (uridine-5′-triphosphate) nucleotides, and GTP (guanosine-5′-triphosphate) nucleotides. In embodiments, the composition includes a plurality of native RNA nucleotides including a plurality of ATP nucleotides, CTP nucleotides, UTP nucleotides, or GTP nucleotides. In embodiments, the composition includes a plurality of ATP nucleotides. In embodiments, the composition includes a plurality of CTP nucleotides. In embodiments, the composition includes a plurality of UTP nucleotides. In embodiments, the composition includes a plurality of GTP nucleotides. In embodiments, the composition consists of a plurality of A ribonucleotides, a plurality of C ribonucleotides, a plurality of U ribonucleotides, and a plurality of G ribonucleotides.

In embodiments, the composition includes a plurality of cleavable site nucleotides. The term “cleavable site nucleotide” refers to a nucleotide that allows for controlled cleavage of the polynucleotide strand following contact with a cleaving agent (e.g., uracil DNA glycosylase (UDG)). Additional examples of cleavable site nucleotides include deoxyuracil triphosphates (dUTPs), deoxy-8-oxo-guanine triphosphates (d-8-oxoGs), methylated nucleotides, or ribonucleotides. In embodiments, the cleavable site nucleotide is dUTP and the cleaving agent is UDG. In embodiments, the cleavable site nucleotide is a ribonucleotide and the cleaving agent is RNase. In embodiments, the cleavable site nucleotide is 8-oxo-7,8-dihydroguanine (8oxoG) and the cleaving agent is formamidopyrimidine DNA glycosylase (Fpg). In embodiments, the cleavable site nucleotide is 5-methylcytosine and the cleaving agent is McrBC.

In embodiments, the cleavable site includes one or more deoxyuracil triphosphates (dUTPs), deoxy-8-oxo-guanine triphosphates (d-8-oxoGs), methylated nucleotides, or ribonucleotides. In embodiments, the cleavable site includes one or more deoxyuracil triphosphates (dUTPs). In embodiments, the cleavable site includes one or more deoxy-8-oxo-guanine triphosphates (d-8-oxoGs). In embodiments, the cleavable site includes one or more methylated nucleotides. In embodiments, the cleavable site includes one or more ribonucleotides. The one or more cleavable sites may include a modified nucleotide, nbonucleotide, or a sequence containing a modified or unmodified nucleotide that is specifically recognized by a cleavage agent. The cleavable site(s) may be deoxyuracil triphosphate (dUTP), deoxy-8-Oxo-guanine triphosphate (d-8-oxoG), or other modified nucleotide(s), such as those described, for example, in US 2012/0238738, which is incorporated herein by reference for all purposes, and include modified ribonucleotides and deoxyribonucleotides including abasic sugar phosphates, inosine, deoxyinosine, 2,6-diamino-4-hydroxy-5-formamidopyrimidine (foramidopyrimidine-guanine, (fapy)-guanine), 8-oxoadenine, 1,N6-ethenoadenine, 3-methyladenine, 4,6-diamino-5-formamidopyrimidine, 5,6-dihydrothymine, 5,6-dihydroxyuracil, 5-formyluracil, 5-hydroxy-5-methylhydanton, 5-hydroxycytosine, 5-hydroxymethylcystosine, 5-hydroxymethyluracil, 5-hydroxyuracil, 6-hydroxy-5,6-dihydrothymine, 6-methyladenine, 7,8-dihydro-8-oxoguanine (8-oxoguanine), 7-methylguanine, aflatoxin B1-fapy-guanine, fapy-adenine, hypoxanthine, methyl-fapy-guanine, methyltartonylurea and thymine glycol. In embodiments, the cleavable site includes an abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. In embodiments, the cleavable site includes one or more ribonucleotides. In embodiments, the cleavable site includes 2 to 5 ribonucleotides. In embodiments, the cleavable site includes one ribonucleotide. In embodiments, the cleavable sites can be cleaved at or near a modified nucleotide or bond by enzymes or chemical reagents, collectively referred to here and in the claims as “cleaving agents.” Examples of cleaving agents include DNA repair enzymes, glycosylases, DNA cleaving endonucleases, or ribonucleases. For example, cleavage at dUTP may be achieved using uracil DNA glycosylase and endonuclease VIII (USER™, NEB, Ipswich, Mass.), as described in U.S. Pat. No. 7,435,572. In embodiments, when the modified nucleotide is a ribonucleotide, the cleavable site can be cleaved with an endoribonuclease. In embodiments, cleaving an extension product includes contacting the cleavable site with a cleaving agent, wherein the cleaving agent includes a reducing agent, sodium periodate, RNase, formamidopyrimidine DNA glycosylase (Fpg), endonuclease, restriction enzyme, or uracil DNA glycosylase (UDG). In embodiments, the cleaving agent is an endonuclease enzyme such as nuclease P1, AP endonuclease, T7 endonuclease, T4 endonuclease IV, Bal 31 endonuclease, Endonuclease I (endo I), Micrococcal nuclease, Endonuclease II (endo VI, exo III), nuclease BAL-31 or mung bean nuclease. In embodiments, the cleaving agent includes a restriction endonuclease, including, for example a type IIS restriction endonuclease. In embodiments, the cleaving agent is an exonuclease (e.g., RecBCD), restriction nuclease, endoribonuclease, exoribonuclease, or RNase (e.g., RNAse 1, II, or 111). In embodiments, the cleaving agent is a restriction enzyme. In embodiments, the cleaving agent includes a glycosylase and one or more suitable endonucleases. In embodiments, cleavage is performed under alkaline (e.g., pH greater than 8) buffer conditions at between 40° C. to 80° C.

In an aspect is provided a method of sequencing a sample polynucleotide including a promoter sequence. In embodiments, the method includes: a) contacting the sample polynucleotide with a polymerase and a composition including a plurality of nucleotides thereby forming a plurality of amplification products; b) contacting the sample with a composition including a plurality of randomer primer oligonucleotides and extending with a polymerase to form a population of different-sized nucleic acid fragments, wherein each of the randomer primer oligonucleotides includes a platform primer binding sequence; c) binding the fragments to immobilized primers on a solid support, and amplifying the fragments to form colonies of immobilized polynucleotide fragments, wherein amplifying includes a plurality of cycles of primer extension, denaturation, and primer hybridization; and d) hybridizing one or more sequencing primers to the colony of immobilized polynucleotide fragments and incorporating one or more nucleotides into the sequencing primer with a polymerase; and detecting the one or more incorporated nucleotides thereby sequencing the sample polynucleotide.

In embodiments, the sample polynucleotide includes a first adapter and a second adapter, wherein the first adapter is a Y-adapter, a hairpin adapter, a blunt-ended adapter, or an adapter including a single-strand overhang and the second adapter is a Y-adapter, a hairpin adapter, a blunt-ended adapter, or an adapter including a single-strand overhang. In embodiments, the sample polynucleotide includes a first adapter and a second adapter, wherein the first adapter is a Y-adapter and the second adapter is a Y-adapter. In embodiments, the sample polynucleotide includes a first adapter and a second adapter, wherein the first adapter is a Y-adapter and the second adapter is a hairpin adapter. In embodiments, the sample polynucleotide includes a first adapter and a second adapter, wherein the first adapter is a hairpin adapter and the second adapter is a Y-adapter. In embodiments, the sample polynucleotide includes a first adapter and a second adapter, wherein the first adapter is a hairpin adapter and the second adapter is a hairpin adapter.

In some embodiments, the adapter is a Y-adapter. In embodiments, a Y-adapter includes a first strand and a second strand where a portion of the first strand (e.g., 3′-portion) is complementary, or substantially complementary, to a portion (e.g., 5′-portion) of the second strand. In embodiments, a Y-adapter includes a first strand and a second strand where a 3′-portion of the first strand is hybridized to a 5′-portion of the second strand. In embodiments, the 3′-portion of the first strand that is substantially complementary to the 5′-portion of the second strand forms a duplex including double stranded nucleic acid. Accordingly, a Y-adapter often includes a first end including a duplex region including a double stranded nucleic acid, and a second end including a forked region including a 5′-arm and a 3′-arm. In some embodiments, a 5′-portion of the first stand (e.g., 5′-arm) and a 3′-portion of the second strand (3′-arm) are not complementary. In embodiments, the first and second strands of a Y-adapter are not covalently attached to each other. In embodiments, the Y-adapter includes (i) a first strand having a 5′-arm and a 3′-portion, and (ii) a second strand having a 3′-arm and a 5′-portion, wherein the 3-portion of the first strand is substantially complementary to the 5′-portion of the second strand, and the 5′-arm of the first strand is not substantially complementary to the 3′-arm of the second strand. In some embodiments, the first adapter includes a sample barcode sequence, a molecular identifier sequence, or both a sample barcode sequence and a molecular identifier sequence. In some embodiments, the first adapter includes a sample barcode sequence (e.g., a 6-10 nucleotide sequence).

In embodiments, ligating includes ligating both the 3′ end and the 5′ end of the duplex region of the first adapter to the double stranded nucleic acid. In embodiments, ligating includes ligating either the 3′ end or the 5′ end of the duplex region of the first adapter to the double stranded nucleic acid. In embodiments, ligating includes ligating the 5′ end of the duplex region of the first adapter to the double stranded nucleic acid and not the 3′ end of the duplex region. In embodiments, the method includes ligating a first adapter to a first end of the double stranded nucleic acid wherein both strands of the double stranded nucleic acid are ligated to the first adapter. In embodiments, the method includes ligating a first adapter to a first end of the double stranded nucleic acid wherein one strand of the double stranded nucleic acid is ligated to the first adapter.

In some embodiments, each strand of a Y-adapter, each of the non-complementary arms of a Y-adapter, or a duplex portion of a Y-adapter has a length independently selected from at least 5, at least 10, at least 15, at least 25, and at least 40 nucleotides. In some embodiments, each strand of a Y-adapter, each of the non-complementary arms of a Y-adapter, or a duplex portion of a Y-adapter has a length in a range independently selected from 15 to 500 nucleotides, 15-250 nucleotides, 15 to 200 nucleotides, 15 to 150 nucleotides, 20 to 100 nucleotides, 20 to 50 nucleotides and 10-50 nucleotides. In embodiments, one or both non-complementary arms of the Y-adapter is about or at least about 10, 15, 20, 25, 30, 35, 40, 45, 50, or more nucleotides in length. In embodiments, one or both non-complementary arms of the Y-adapter is about or at least about 20 nucleotides in length. In embodiments, one or both non-complementary arms of the Y-adapter is about or at least about 30 nucleotides in length. In embodiments, one or both non-complementary arms of the Y-adapter is about or at least about 40 nucleotides in length. In embodiments, the duplex portion of a Y-adapter is about or at least about 5, 10, 15, 20, 25, 30, or more nucleotides in length. In embodiments, the duplex portion of a Y-adapter is about 5-50, 5-25, or 10-15 nucleotides in length. In embodiments, the duplex portion of a Y-adapter is about or at least about 10 nucleotides in length. In embodiments, the duplex portion of a Y-adapter is about or at least about 15 nucleotides in length. In embodiments, the duplex portion of a Y-adapter is about or at least about 12 nucleotides in length. In embodiments, the duplex portion of a Y-adapter is about or at least about 20 nucleotides in length.

In some embodiments, a Y-adapter includes a first end including a duplex region including a double stranded nucleic acid, and a second end including a forked region, where the first end is configured for ligation to an end of a double stranded nucleic acid (e.g., a nucleic acid fragment, e.g., a library insert). In embodiments, a duplex end of a Y-adapter includes a 5′-overhang or a 3′-overhang that is complementary to a 3′-overhang or a 5′-overhang of an end of a double stranded nucleic acid. In some embodiments, a duplex end of a Y-adapter includes a blunt end that can be ligated to a blunt end of a double stranded nucleic acid. In certain embodiment, a duplex end of a Y-adapter includes a 5′-end that is phosphorylated.

In some embodiments, the first and/or second adapter (e.g., one or both strands of a Y-adapter) include one or more of a primer binding site, a capture nucleic acid binding site (e.g., a nucleic acid sequence complementary to a capture nucleic acid), a UMI, a sample barcode, a sequencing adapter, a label, a binding motif, the like or combinations thereof. In some embodiments, a non-complementary portion (e.g., 5′-arm and/or 3′-arm) of a Y-adapter includes one or more of a primer binding site, a capture nucleic acid binding site (e.g., a nucleic acid sequence complementary to a capture nucleic acid), a UMI, a sample barcode, a sequencing adapter, a label, a binding motif the like or combinations thereof. In certain embodiments, a non-complementary portion of a Y-adapter includes a primer binding site. In certain embodiments, a non-complementary portion of a Y-adapter includes a binding site for a capture nucleic acid. In certain embodiments, a non-complementary portion of a Y-adapter includes a primer binding site and a UMI. In certain embodiments, a non-complementary portion of a Y-adapter includes a binding motif. In embodiments, the first and/or second adapter (e.g., one or both strands of a Y-adapter) does not include a UMI or sample barcode.

In embodiments, a complementary strand (e.g., a 3′-portion or 5′-portion) of a Y-adapter includes a primer binding site. In certain embodiments, a complementary strand (e.g., a 3′-portion or 5′-portion) of a Y-adapter includes a binding site for a capture nucleic acid. In certain embodiments, a complementary strand (e.g., a 3′-portion or 5′-portion) of a Y-adapter includes a primer binding site and a UMI. In certain embodiments, a complementary strand (e.g., a 3′-portion or 5′-portion) of a Y-adapter includes a binding motif.

In some embodiments, each of the non-complementary portions (i.e., arms) of a Y-adapter independently have a predicted, calculated, mean, average or absolute melting temperature (Tm) that is greater than 50° C., greater than 55° C., greater than 60° C., greater than 65° C., greater than 70° C. or greater than 75° C. In some embodiments, each of the non-complementary portions of a Y-adapter independently have a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm is about or at least about 70° C. In embodiments, the Tm is about or at least about 75° C. In embodiments, the Tm is about or at least about 80° C. In embodiments, the Tm is a calculated Tm. Tm's are routinely calculated by those skilled in the art, such as by commercial providers of custom oligonucleotides. In embodiments, the Tm for a given sequence is determined based on that sequence as an independent oligo. In embodiments, Tm is calculated using web-based algorithms, such as Primer3 and Primer3Plus (www.bioinformatics.nl/cgi-bin/primer3plus/primer3plus.cgi) using default parameters. The Tm of a non-complementary portion of a Y-adapter can be changed (e.g., increased) to a desired Tm using a suitable method, for example by changing (e.g., increasing) GC content, changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, each of the non-complementary portion of a Y-adapter independently includes one or more modified nucleotides, nucleotide analogues and/or modified nucleotides bonds.

In some embodiments, each of the non-complementary portions of a Y-adapter independently includes a GC content of greater than 40%, greater than 50%, greater than 55%, greater than 60% greater than 65% or greater than 70%. In certain embodiments, each of the non-complementary portions of a Y-adapter independently includes a GC content in a range of 40-100%, 50-100%, 60-100% or 70-100%. In embodiments, one or both non-complementary portions of a Y-adapter have a GC content of about or more than about 40%. In embodiments, one or both non-complementary portions of a Y-adapter have a GC content of about or more than about 50%. In embodiments, one or both non-complementary portions of a Y-adapter have a GC content of about or more than about 60%. Non-base modifiers can also be incorporated into a non-complementary portion of a Y-adapter to increase Tm, non-limiting examples of which include a minor grove binder (MGB), spermine, G-clamp, a Uaq anthraquinone cap, the like or combinations thereof.

In certain embodiments, a duplex region of a Y-adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of a duplex region of the Y-adapter is about or more than about 30° C. In embodiments, the Tm of a duplex region of the Y-adapter is about or more than about 35° C. In embodiments, the Tm of a duplex region of the Y-adapter is about or more than about 40° C. In embodiments, the Tm of a duplex region of the Y-adapter is about or more than about 45° C. In embodiments, the Tm of a duplex region of the Y-adapter is about or more than about 50° C.

In some embodiments, the adapter is hairpin adapter. In embodiments, a hairpin adapter includes a single nucleic acid strand including a stem-loop structure. A hairpin adapter can be any suitable length. In some embodiments, a hairpin adapter is at least 40, at least 50, or at least 100 nucleotides in length. In some embodiments, a hairpin adapter has a length in a range of 45 to 500 nucleotides, 75-500 nucleotides, 45 to 250 nucleotides, 60 to 250 nucleotides or 45 to 150 nucleotides. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, the second adapter includes a sample barcode sequence, a molecular identifier sequence, or both a sample barcode sequence and a molecular identifier sequence. In some embodiments, the second adapter includes a sample barcode sequence.

In some embodiments, a duplex region or stem portion of a hairpin adapter includes an end that is configured for ligation to an end of double stranded nucleic acid (e.g., a nucleic acid fragment, e.g., a library insert). In embodiments, an end of a duplex region or stem portion of a hairpin adapter includes a 5′-overhang or a 3′-overhang that is complementary to a 3′-overhang or a 5′-overhang of one end of a double stranded nucleic acid. In some embodiments, an end of a duplex region or stem portion of a hairpin adapter includes a blunt end that can be ligated to a blunt end of a double stranded nucleic acid. In certain embodiment, an end of a duplex region or stem portion of a hairpin adapter includes a 5′-end that is phosphorylated. In some embodiments, a stem portion of a hairpin adapter is at least 15, at least 25, or at least 40 nucleotides in length. In some embodiments, a stem portion of a hairpin adapter has a length in a range of 15 to 500 nucleotides, 15-250 nucleotides, 15 to 200 nucleotides, 15 to 150 nucleotides. 20 to 100 nucleotides or 20 to 50 nucleotides.

In embodiments, ligating includes ligating both the 3′ end and the 5′ end of the duplex region of the second adapter to the double stranded nucleic acid. In embodiments, ligating includes ligating either the 3′ end or the 5′ end of the duplex region of the second adapter to the double stranded nucleic acid. In embodiments, ligating includes ligating the 5′ end of the duplex region of the second adapter to the double stranded nucleic acid and not the 3′ end of the duplex region.

In some embodiments, the loop of a hairpin adapter includes one or more of a primer binding site, a capture nucleic acid binding site (e.g., a nucleic acid sequence complementary to a capture nucleic acid), a UMI, a sample barcode, a sequencing adapter, a label, the like or combinations thereof. In certain embodiments, a loop of a hairpin adapter includes a primer binding site. In certain embodiments, a loop of a hairpin adapter includes a primer binding site and a UMI. In certain embodiments, a loop of a hairpin adapter includes a binding motif.

In some embodiments, the loop of a hairpin adapter has a predicted, calculated, mean, average or absolute melting temperature (Tm) that is greater than 50° C., greater than 55° C., greater than 60° C., greater than 65° C., greater than 70° C. or greater than 75° C. In some embodiments, a loop of a hairpin adapter has a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm of the loop is about 65° C. In embodiments, the Tm of the loop is about 75° C. In embodiments, the Tm of the loop is about 85° C. The Tm of a loop of a hairpin adapter can be changed (e.g., increased) to a desired Tm using a suitable method, for example by changing (e.g., increasing GC content), changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, a loop of a hairpin adapter includes one or more modified nucleotides, nucleotide analogues and/or modified nucleotides bonds.

In some embodiments, the loop of a hairpin adapter independently includes a GC content of greater than 40%, greater than 50%, greater than 55%, greater than 60% greater than 65% or greater than 70%. In certain embodiments, a loop of a hairpin adapter independently includes a GC content in a range of 40-100%, 50-100%, 60-100% or 70-100%. In embodiments, the loop has a GC content of about or more than about 40%. In embodiments, the loop has a GC content of about or more than about 50%. In embodiments, the loop has a GC content of about or more than about 60%. Non-base modifiers can also be incorporated into a loop of a hairpin adapter to increase Tm, non-limiting examples of which include a minor grove binder (MGB), spermine, G-clamp, a Uaq anthraquinone cap, the like or combinations thereof. A loop of a hairpin adapter can be any suitable length. In some embodiments, a loop of a hairpin adapter is at least 15, at least 25, or at least 40 nucleotides in length. In some embodiments, a hairpin adapter has a length in a range of 15 to 500 nucleotides, 15-250 nucleotides, 20 to 200 nucleotides, 30 to 150 nucleotides or 50 to 100 nucleotides.

In certain embodiments, a duplex region or stem region of a hairpin adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of the stem region is about or more than about 35° C. In embodiments, the Tm of the stem region is about or more than about 40° C. In embodiments, the Tm of the stem region is about or more than about 45° C. In embodiments, the Tm of the stem region is about or more than about 50° C.

In embodiments, the first adapter, the second adapter, or both the first adapter and the second adapter include a barcode sequence (alternatively referred to herein as a UMI). In embodiments, each adapter includes, from 5′ to 3′, a barcode sequence, a primer binding site, and a promoter sequence.

In embodiments, the sample polynucleotide includes a promoter sequence. In embodiments, step a) includes contacting the sample polynucleotide with a composition including a plurality of nucleotides and a a primer complementary to said promoter sequence, and transcribing the sample polynucleotide with an RNA polymerase thereby forming a plurality of amplification products.

In embodiments, each adapter includes (i) a first strand including, from 5′ to 3′, a barcode sequence, a first primer binding sequence, a second primer binding sequence, and a promoter sequence; and (ii) a second strand including, from 3′ to 5, a sequence complementary to the barcode sequence, and a sequence complementary to the first primer binding sequence. In embodiments, each adapter includes, from 5′ to 3, a barcode sequence, a primer binding sequence, a promoter sequence, a cleavable site, and a sequence complementary to the barcode sequence. In embodiments, each adapter includes, from 5′ to 3′, a first barcode sequence, a primer binding site, a promoter sequence, and a second barcode sequence.

In embodiments, each adapter includes (i) a first strand including, from 5′ to 3′, a constant region, a barcode sequence, a first primer binding sequence, a second primer binding sequence, and a promoter sequence; and (ii) a second strand including, from 3′ to 5′, a sequence complementary to the constant region, a sequence complementary to the barcode sequence, and a sequence complementary to the first primer binding sequence. In embodiments, each adapter includes, from 5′ to 3′, a barcode sequence, a constant region, a primer binding sequence, a promoter sequence, a cleavable site, a sequence complementary to the constant region, and a sequence complementary to the barcode sequence. In embodiments, each adapter includes, from 5′ to 3′, a constant region, a first barcode sequence, a primer binding site, a promoter sequence, and a second barcode sequence, and a sequence complementary to the constant region.

In embodiments, the first adapter and the second adapter include identical barcode sequences. In embodiments, the first adapter and the second adapter include unique barcode sequences, relative to each other.

In embodiments, each barcode sequence is selected from a set of barcode sequences represented by a random or partially random sequence. In embodiments, each barcode sequence is selected from a set of barcode sequences represented by a random sequence. In embodiments, each barcode sequence is selected from a set of barcode sequences represented by a partially random sequence. In embodiments, each barcode sequence includes a random sequence. In embodiments, the random sequence excludes a subset of sequences, where the excluded subset includes sequences with three or more identical consecutive nucleotides. In embodiments, the excluded subset includes sequences with three identical consecutive nucleotides. In embodiments, the excluded subset includes sequences with four identical consecutive nucleotides (e.g., GGGG). In embodiments, the excluded subset includes sequences with five identical consecutive nucleotides (e.g., GGGGG).

In embodiments, the barcode sequences each include about 5 to about 20 nucleotides, or about 10 to about 20 nucleotides. In embodiments, the barcode sequence includes about 5 to about 20 nucleotides. In embodiments, the barcode sequence includes about 5 nucleotides. In embodiments, the barcode sequence includes about 6 nucleotides. In embodiments, the barcode sequence includes about 7 nucleotides. In embodiments, the barcode sequence includes about 8 nucleotides. In embodiments, the barcode sequence includes about 9 nucleotides. In embodiments, the barcode sequence includes about 10 nucleotides. In embodiments, the barcode sequence includes about 11 nucleotides. In embodiments, the barcode sequence includes about 12 nucleotides. In embodiments, the barcode sequence includes about 13 nucleotides. In embodiments, the barcode sequence includes about 14 nucleotides. In embodiments the barcode sequence includes about 15 nucleotides. In embodiments, the barcode sequence includes about 16 nucleotides. In embodiments, the barcode sequence includes about 17 nucleotides. In embodiments, the barcode sequence includes about 18 nucleotides. In embodiments, the barcode sequence includes about 19 nucleotides. In embodiments, the barcode sequence includes about 20 nucleotides.

In embodiments, each barcode sequence differs from every other barcode sequence by at least two nucleotide positions. In embodiments, each barcode sequence differs from every other barcode sequence by at least three nucleotide positions. In embodiments, each barcode sequence differs from every other barcode sequence by at least four nucleotide positions. In embodiments, each barcode sequence differs from every other barcode sequence by at least five nucleotide positions.

In embodiments, the randomer primer oligonucleotides include, from 3′ to 5′, a non-targeted template hybridization sequence and a platform primer sequence. In embodiments, the non-targeted template hybridization sequence is a random sequence. In embodiments, the overall length of the randomer primer oligonucleotide is about 25 to about 70 nucleotides (e.g., the non-targeted template hybridization sequence is about 4 to about 30 nucleotides in length and the platform primer sequence is about 20 to about 40 nucleotides in length). In embodiments, the overall length of the randomer primer oligonucleotide is about 25 to about 35 nucleotides. In embodiments, the overall length of the randomer primer oligonucleotide is about 35 to about 45 nucleotides. In embodiments, the overall length of the randomer primer oligonucleotide is about 45 to about 55 nucleotides. In embodiments, the overall length of the randomer primer oligonucleotide is about 55 to about 70 nucleotides. In embodiments, the overall length of the randomer primer oligonucleotide is about 25, about 30, about 35, about 40, about 45, about 50, about 55, about 60, about 65, or about 70 nucleotides. In embodiments, the non-targeted template hybridization sequence is about 4 to about 30 nucleotides in length. In embodiments, the non-targeted template hybridization sequence is about 4 to about 8 nucleotides in length. In embodiments, the non-targeted template hybridization sequence is about 8 to about 12 nucleotides in length. In embodiments, the non-targeted template hybridization sequence is about 12 to about 16 nucleotides in length. In embodiments, the non-targeted template hybridization sequence is about 16 to about 20 nucleotides in length. In embodiments, the non-targeted template hybridization sequence is about 20 to about 30 nucleotides in length. In embodiments, the non-targeted template hybridization sequence is at least 4, at least 5, at least 6, at least 7, at least 8, at least 9, at least 10, at least 12, at least 15, at least 18, at least 20, at least 25, or at least 30 nucleotides in length. In embodiments, the platform primer sequence is about 20 to about 40 nucleotides in length. In embodiments, the platform primer sequence is about 20, about 25, about 30, about 35, or about 40 nucleotides in length.

In embodiments, the sample polynucleotide is a double-stranded polynucleotide. In embodiments, the double-stranded polynucleotide includes genomic DNA, complementary DNA (cDNA), cell-free DNA (cfDNA), messenger RNA (mRNA), transfer RNA (tRNA), nbosomal RNA (rRNA), cell-free RNA (cfRNA), or noncoding RNA (ncRNA). In embodiments, the sample polynucleotide is a single-stranded polynucleotide.

In embodiments, the double-stranded polynucleotide is about 100 to 1000 nucleotides in length. In embodiments, the double-stranded polynucleotide is about 350 nucleotides in length. In embodiments, the double-stranded polynucleotide is about 10, 20, 50, 100, 150, 200, 300, or 500 nucleotides in length. The double-stranded polynucleotide molecules can vary length, such as about 100-300 nucleotides long, about 300-500 nucleotides long, or about 500-1000 nucleotides long. In embodiments, the double-stranded polynucleotide molecular is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides. In embodiments, the double-stranded polynucleotide molecule is about 150 nucleotides. In embodiments, the double-stranded polynucleotide is about 100-1000 nucleotides long. In embodiments, the double-stranded polynucleotide is about 100-300 nucleotides long. In embodiments, the double-stranded polynucleotide is about 300-500 nucleotides long. In embodiments, the double-stranded polynucleotide is about 500-1000 nucleotides long. In embodiments, the double-stranded polynucleotide molecule is about 100 nucleotides. In embodiments, the double-stranded polynucleotide molecule is about 300 nucleotides. In embodiments, the double-stranded polynucleotide molecule is about 500 nucleotides. In embodiments, the double-stranded polynucleotide molecule is about 1,000 nucleotides. In embodiments, the double-stranded polynucleotide molecule is about 2,000 nucleotides. In embodiments, the double-stranded polynucleotide molecule is about 3,000 nucleotides. In embodiments, the double-stranded polynucleotide molecule is about 4,000 nucleotides. In embodiments, the double-stranded polynucleotide molecule is about 5,000 nucleotides. In embodiments, the double-stranded polynucleotide molecule is about 6,000 nucleotides. In embodiments, the double-stranded polynucleotide molecule is about 7,000 nucleotides. In embodiments, the double-stranded polynucleotide molecule is about 8,000 nucleotides. In embodiments, the double-stranded polynucleotide molecule is about 9,000 nucleotides. In embodiments, the double-stranded polynucleotide molecule is about 10,000 nucleotides. In embodiments, the double-stranded polynucleotide molecule is about 10,000 to about 50,000 nucleotides. In embodiments, the double-stranded polynucleotide molecule is about 20,000 nucleotides. In embodiments, the double-stranded polynucleotide molecule is about 30,000 nucleotides. In embodiments, the double-stranded polynucleotide molecule is about 40,000 nucleotides. In embodiments, the double-stranded polynucleotide molecule is about 50,000 nucleotides.

In embodiments the double-stranded polynucleotide (e.g., genomic template DNA) is first treated to form single-stranded linear nucleic acid fragments (e.g., ranging in length from about 50 to about 600 nucleotides). Treatment typically entails fragmentation, such as by chemical fragmentation, enzymatic fragmentation, or mechanical fragmentation, followed by denaturation to produce single-stranded DNA fragments. In embodiments, the double-stranded polynucleotide includes an adapter. The adapter may have other functional elements including tagging sequences (i.e., a barcode), attachment sequences, palindromic sequences, restriction sites, sequencing primer binding sites, functionalization sequences, and the like. Barcodes can be of any of a variety of lengths. In embodiments, the primer includes a barcode that is 10-50, 20-30, or 4-12 nucleotides in length. In embodiments, the adapter includes a primer binding sequence that is complementary to at least a portion of a primer (e.g., a sequencing primer). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length. In embodiments the double-stranded polynucleotide is cfDNA.

In embodiments, the double-stranded polynucleotide includes known adapter sequences on the 5′ and 3′ ends.

In embodiments, the reverse transcriptase is a strand-displacing reverse transcriptase. In embodiments, the strand-displacing reverse transcriptase is a Moloney murine leukemia virus M-MLV reverse transcriptase, or variant thereof. In embodiments, the strand-displacing reverse transcriptase is an avian myeloblastosis virus (AMV) reverse transcriptase, or variant thereof, In embodiments, the strand-displacing reverse transcriptase is a human immunodeficiency virus 1 (HIV-1) reverse transcriptase, or variant thereof.

In embodiments, the promoter sequence is a T3 RNA polymerase promoter sequence, T5 RNA polymerase promoter sequence, or T7 RNA polymerase promoter sequence. In embodiments, the promoter sequence is a T3 RNA polymerase promoter sequence (e.g, from 5′ to 3′: AATTAACCCTCACTAAAG (SEQ ID NO: 1)). In embodiments, the promoter sequence is a T5 RNA polymerase promoter sequence (e.g., from 5′ to 3′: TCATAAAAAATTTATTTGCT (SEQ ID NO: 2)). In embodiments, the promoter sequence is a T7 RNA polymerase promoter sequence (e.g., from 5′ to 3′: TAATACGACTCACTATAGGGAGA (SEQ ID NO: 3)).

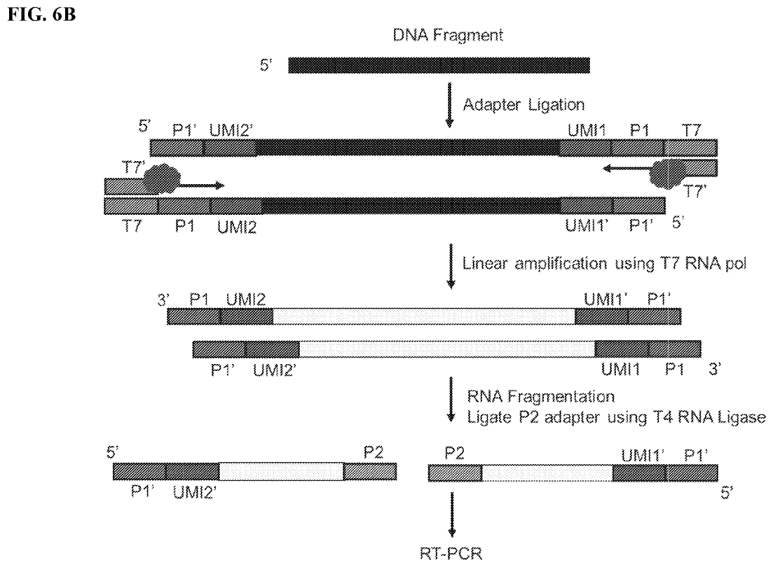

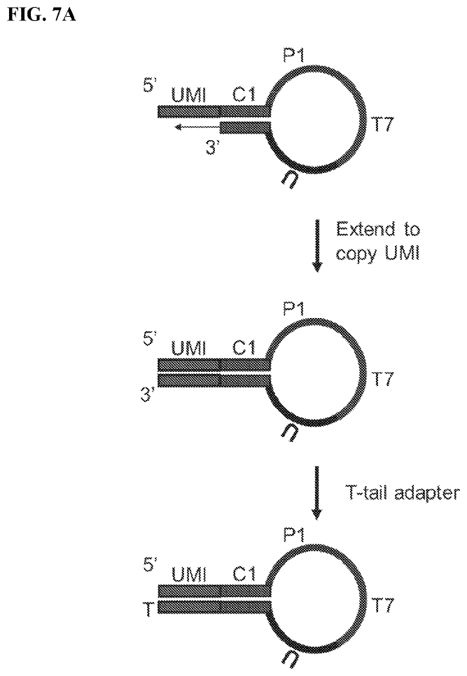

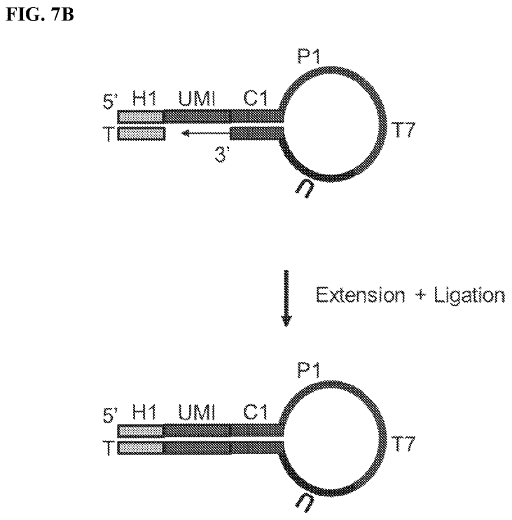

In embodiments, prior to step a), the method further includes contacting each adapter with a polymerase and extending the 3′ end of the adapter to generate the sequence complementary to the barcode sequence. For example, as described in FIG. 6A, a primer is hybridized at the 5′ end of a UMI sequence (e.g., hybridized to the P1 primer binding sequence) and extended using a polymerase with exonuclease activity, such that the UMI sequence is copied, followed by T-tailing (e.g., with Taq polymerase) to leave a T 3′ overhang. In embodiments, the adapter includes from 5′ to 3′ a UMI sequence (e.g., UMI1), a primer binding sequence (e.g., P1), and a promoter sequence (e.g., a T7 promoter sequence). Alternatively, as described in FIG. 7A, the 3′ end of a hairpin adapter is extended using a polymerase with exonuclease activity, such that the UMI sequence is copied, followed by T-tailing to leave a T 3′-overhang. In embodiments, the adapter includes from 5′ to 3′ a UMI sequence (e.g., UMI1), a constant (or stem) region (e.g., C1), a primer binding sequence (e.g., P1), a promoter sequence (e.g., a T7 promoter sequence), a cleavable site (e.g., a uracil nucleotide), and a sequence complementary to the constant region.

In embodiments, the method further includes, prior to step (b), fragmenting the plurality of amplification products to generate a plurality of polynucleotide fragments including 3′ ends, and ligating an adapter sequence to the 3′ end of each of the polynucleotide fragments. In embodiments, the adapter includes single-stranded RNA. In embodiments, the adapter sequence is ligated onto a single-stranded nucleic acid with a ligase, wherein the ligase is T4 RNA ligase.

In embodiments, prior to forming a population of RNA nucleic acid fragments, an aliquot (e.g., a portion of the total amount) including the sample polynucleotide including at least a first adapter is retained. In embodiments, prior to forming a population of RNA nucleic acid fragments, an aliquot including the sample polynucleotide is retained. In embodiments, prior to forming a population of RNA nucleic acid fragments, an aliquot including the sample polynucleotide is retained, wherein the sample polynucleotide includes at least a first adapter. In embodiments, prior to forming a population of RNA nucleic acid fragments, an aliquot including the sample polynucleotide is retained, wherein the sample polynucleotide includes a first adapter and a second adapter. In embodiments, the retained aliquot does not include any RNA fragment polynucleotides.

In embodiments, prior to forming a population of different-sized nucleic acid fragments, an aliquot (e.g., a portion of the total amount) including the sample polynucleotide including at least a first adapter is retained. In embodiments, prior to forming a population of different-sized nucleic acid fragments, an aliquot including the sample polynucleotide is retained. In embodiments, prior to forming a population of different-sized nucleic acid fragments, an aliquot including the sample polynucleotide is retained, wherein the sample polynucleotide includes at least a first adapter. In embodiments, prior to forming a population of different-sized nucleic acid fragments, an aliquot including the sample polynucleotide is retained, wherein the sample polynucleotide includes a first adapter and a second adapter. In embodiments, the retained aliquot does not include any fragment polynucleotides.

In embodiments, forming a plurality of amplification products includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, generating a double-stranded amplification product includes a bridge polymerase chain reaction (bPCR) amplification. In embodiments, generating a double-stranded amplification product includes a thermal bridge polymerase chain reaction (t-bPCR) amplification. In embodiments, generating a double-stranded amplification product includes a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions.

In embodiments, forming a plurality of amplification products includes bridge amplification; for example, as exemplified by the disclosures of U.S. Pat. Nos. 5,641,658; 7,115,400; 7,790,418; U.S. Patent Publ. No. 2008/0009420, each of which is incorporated herein by reference in its entirety. In general, bridge amplification uses repeated steps of annealing of primers to templates, primer extension, and separation of extended primers from templates. Because the forward and reverse primers are attached to the solid support, the extension products released upon separation from an initial template are also attached to the solid support. Both strands are immobilized on the solid support at the 5′ end, preferably via a covalent attachment. The 3′ end of an amplification product is then permitted to anneal to a nearby reverse primer, forming a “bridge” structure. The reverse primer is then extended to produce a further template molecule that can form another bridge. During bridge PCR, additional chemical additives may be included in the reaction mixture, in which the DNA strands are denatured by flowing a denaturant over the DNA, which chemically denatures complementary strands. This is followed by washing out the denaturant and reintroducing a polymerase in buffer conditions that allow primer annealing and extension.

In embodiments, forming a plurality of amplification products includes amplifying the template polynucleotide or complement thereof on a solid support including a plurality of primers attached to the solid support, wherein the plurality of primers include a plurality of forward primers with complementarity to the template polynucleotide and a plurality of reverse primers with complementarity to a complement of the template polynucleotide, and the amplifying includes a plurality of cycles of strand denaturation, primer hybridization, and primer extension.

In embodiments, the plurality of strand denaturation cycles are different for one or more cycles, wherein the initial denaturation cycle is maintained at different conditions from the remaining denaturation cycles. For example, in embodiments, the initial denaturation cycle is at about 85° C.-95° C. for about 1 minute to about 10 minutes, whereas denaturation in the remaining cycles is different (e.g., about 85° C. for about 15-30 sec). In embodiments, the initial denaturation is maintained at about 85° C.-95° C. for about 5 minutes to about 10 minutes. In embodiments, the initial denaturation is maintained at 90° C.-95° C. for about 1 to 10 minutes. In embodiments, the initial denaturation is maintained at 80° C.-85° C. for about 1 to 10 minutes. In embodiments, the initial denaturation is maintained at 85° C.-90° C. for about 1 to 10 minutes. In embodiments, the initial denaturation is maintained at about 85° C.-95° C. for about 1 minutes to about 10 minutes. In embodiments, the initial denaturation is maintained at about 95° C. for about 5 minutes to about 10 minutes. In embodiments, the initial denaturation is maintained at about 85° C.-95° C. for about 5 minutes to about 10 minutes.

In embodiments, forming a plurality of amplification products includes a thermal bridge polymerase chain reaction (t-bPCR) amplification. In embodiments, the plurality of cycles includes thermally cycling between (i) about 85° C. for about 15-30 sec for denaturation, and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer.

In embodiments, forming a plurality of amplification products includes chemical bridge polymerase chain reaction (c-bPCR) amplification. In embodiments, forming a plurality of amplification products includes denaturation using a chemical denaturant. In embodiments, forming a plurality of amplification products includes denaturation using acetic acid, hydrochloric acid, nitric acid, formamide, guanidine, sodium salicylate, sodium hydroxide, dimethyl sulfoxide (DMSO), propylene glycol, urea, or a mixture thereof. In embodiments, the chemical denaturant is sodium hydroxide or formamide. In embodiments, forming a plurality of amplification products includes thermal bridge polymerase chain reaction (t-bPCR) amplification. In embodiments, forming a plurality of amplification products includes chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a significantly lower concentration than traditional chemical bridge polymerase chain reactions.

In embodiments, forming a plurality of amplification products includes fluidic cycling between an extension mixture that includes a polymerase and dNTPs, and a chemical denaturant. In embodiments, the polymerase is a strand-displacing polymerase or a non-strand displacing polymerase. In embodiments, the solutions are thermally cycled between about 40° C. to about 65° C. during fluidic cycling of the extension mixture and the chemical denaturant. For example, the extension cycle is maintained at a temperature of 55° C.-65° C., followed by a denaturation cycle that is maintained at a temperature of 40° C.-65° C., or by a denaturation step in which the temperature starts at 60° C.-65° C. and is ramped down to 40° C. prior to exchanging the reagent. In embodiments, step (b) includes modulating the reaction temperature prior to initiating the next cycle. In embodiments, the denaturation cycle and/or the extension cycle is maintained at a temperature for a sufficient amount of time, and prior to starting the next cycle the temperature is modulated (e.g., increased relative to the starting temperature or reduced relative to the starting temperature). In embodiments, the denaturation cycle is performed at a temperature of 60° C.-65° C. for about 5-45 sec, then the temperature is reduced (e.g., lowered to about 40° C.) before starting an extension cycle (i.e., before introducing an extension mixture). Lowering the temperature, even in the presence of a chemical denaturant, facilitates primer hybridization in the subsequent step when the amplicons are exposed to conditions that promote hybridization. In embodiments, the extension cycle is performed at a temperature of 50° C.-60° C. for about 0.5-2 minutes, then the temperature is increased (e.g., raised to between about 60° C. to about 70° C., or to about 65° C. to about 72° C.) after introducing the extension mixture. In embodiments, the cycling between the extension mixture and the chemical denaturant is performed at least 5, at least 10, at least 20, at least 30, at least 40, at least 50, at least 75, at least 100, or at least 200 times. In embodiments, the cycling between the extension mixture and the chemical denaturant is performed about 5, about 10, about 20, about 30, about 40, about 50, about 75, about 100, or about 200 times. In embodiments, the cycling between the extension mixture and the chemical denaturant is performed a total of 5, 10, 20, 30, 40, 50, 75, 100, 200, or more times. In embodiments, the fluidic cycling is performed in the presence of about 2 to about 15 mM Mg2+. In embodiments, the fluidic cycling is performed in the presence of about 2, about 3, about 4, about 5, about 6, about 7, about 8, about 9, about 10, about 11, about 12, about 13, about 14, or about 15 mM Mg+.

In embodiments, forming a plurality of amplification products includes a plurality of strand denaturation cycles, wherein the initial denaturation cycle is at different conditions from the remaining denaturation cycles. For example, in embodiments, the initial denaturation cycle is at about 85° C.-95° C. for about 1 minute to about 10 minutes, whereas denaturation in the remaining cycles is different (e.g. about 85° C. for about 15-30 sec). In embodiments, forming a plurality of amplification products includes an initial denaturation at about 85° C.-95° C. for about 5 minutes to about 10 minutes. In embodiments, forming a plurality of amplification products includes an initial denaturation at 90° C.-95° C. for about 1 to 10 minutes. In embodiments, forming a plurality of amplification products includes an initial denaturation at 80° C.-85° C. for about 1 to 10 minutes. In embodiments, forming a plurality of amplification products includes an initial denaturation at 85° C.-90° C. for about 1 to 10 minutes.

In embodiments, the plurality of cycles includes thermally cycling between (i) about 80° C. to 90° C. for denaturation, and (ii) about 55° C. to about 65° C. for annealing/extension of the primer. In embodiments, the plurality of cycles includes thermally cycling between (i) about 85° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer. In embodiments, the plurality of cycles includes thermally cycling between (i) less than 80° C. (e.g., 70 to 80° C.) for denaturation, and (ii) about 55° C. to about 65° C. for annealing/extension of the primer. In embodiments, the plurality of cycles includes thermally cycling between (i) about 70° C. for denaturation, and (ii) about 65° C. for annealing/extension of the primer. In embodiments, the plurality of cycles includes thermally cycling between (i) about 75° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer.

In embodiments, the plurality of cycles includes thermally cycling between (i) about 85° C. for less than 1 minute for denaturation, and (ii) about 65° C. for about 1 to 2 minutes for annealing/extension of the primer. In embodiments, the plurality of cycles includes thermally cycling between (i) about 85° C. for less than 1 minute for denaturation, and (ii) about 60° C. to about 65° C. for about 1 minute for annealing/extension of the primer. In embodiments, the plurality of cycles includes thermally cycling between (i) about 85° C. for about 15-30 sec for denaturation and (ii) about 65° C. for about 1 minute for annealing/extension of the primer. In embodiments, the plurality of cycles includes thermally cycling between (i) about 85° C. for about 30 sec for denaturation and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer. In embodiments, the plurality of cycles includes thermally cycling between (i) about 85° C. for about 15-30 sec for denaturation, and (ii) about 65° C. for about 1 minute for annealing/extension of the primer.

In embodiments, the plurality of denaturation steps is at a temperature of about 80° C.-95° C. In embodiments, the plurality of denaturation steps is at a temperature of about 80° C.-90° C. In embodiments, the plurality of denaturation steps is at a temperature of about 85° C.-90° C. In embodiments, the plurality of denaturation steps is at a temperature of about 81° C., 82° C., 83° C., 84° C. 85° C., 86° C., 87° C., 88° C., 89° C., or about 90° C. In embodiments, the plurality of denaturation steps is at a temperature of about 70° C.-85° C. In embodiments, the plurality of denaturation steps is at a temperature of about 70° C.-80° C. In embodiments, the plurality of denaturation steps is at a temperature of about 75° C.-80° C. In embodiments, the plurality of denaturation steps is at a temperature of about 70° C., 71° C., 72° C., 73° C., 74° C., 75° C., 76° C., 77° C., 78° C. 79° C., or about 80° C. In embodiments, the annealing/extension of the primer cycle is at a temperature of about 55° C., 56° C., 57° C., 58° C., 59° C., 60° C., 61° C., 62° C., 63° C., 64° C., or about 65° C.

In embodiments, the plurality of cycles includes thermally cycling between (i) about 80° C. to 90° C. for denaturation, and (ii) about 55° C. to about 65° C. for annealing/extension of the primer. In embodiments, the plurality of cycles includes thermally cycling between (i) about 85° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer. In embodiments, the plurality of cycles includes thermally cycling between (i) less than 80° C. (e.g., 70 to 80° C.) for denaturation, and (ii) about 55° C. to about 65° C. for annealing/extension of the primer. In embodiments, the plurality of cycles includes thermally cycling between (i) about 70° C. for denaturation, and (ii) about 65° C. for annealing/extension of the primer. In embodiments, the plurality of cycles includes thermally cycling between (i) about 75° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer.

In embodiments, the plurality of cycles includes thermally cycling between (i) about 85° C. for less than 1 minute for denaturation, and (ii) about 65° C. for about 1 to 2 minutes for annealing/extension of the primer. In embodiments, the plurality of cycles includes thermally cycling between (i) about 85° C. for less than 1 minute for denaturation, and (ii) about 60° C. to about 65° C. for about 1 minute for annealing/extension of the primer. In embodiments, the plurality of cycles includes thermally cycling between (i) about 85° C. for about 15-30 sec for denaturation and (ii) about 65° C. for about 1 minute for annealing/extension of the primer. In embodiments, the plurality of cycles includes thermally cycling between (i) about 85° C. for about 30 sec for denaturation and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer. In embodiments, the plurality of cycles includes thermally cycling between (i) about 85° C. for about 15-30 sec for denaturation, and (ii) about 65° C. for about 1 minute for annealing/extension of the primer. In embodiments, the temperature and duration for the annealing of the primer and the extension of the primer are different. In embodiments, the plurality of cycles includes thermally cycling between (i) about 90° C. to 95° C. for about 15 to 30 sec for denaturation and (ii) about 55° C. to about 65° C. for about 30 to 60 seconds for annealing and about 65° C. to 70° C. for about 30 to 60 seconds for extension of the primer. In embodiments, the plurality of denaturation steps is at a temperature of about 80° C.-95° C. In embodiments, the plurality of denaturation steps is at a temperature of about 80° C.-90° C. In embodiments, the plurality of denaturation steps is at a temperature of about 85° C.-90° C. In embodiments, the plurality of denaturation steps is at a temperature of about 81° C., 82° C., 83° C., 84° C., 85° C., 86° C., 87° C., 88° C., 89° C., or about 90° C. In embodiments, the plurality of denaturation steps is at a temperature of about 91° C., 92° C., 93° C., 94° C., 95° C., 96° C., 97° C., 98° C., or about 99° C. In embodiments, the plurality of denaturation steps is at a temperature of about 87° C., 88° C., 89° C., 90° C., 91° C., 92° C., 93° C., 94° C., or about 95° C. In embodiments, the plurality of denaturation steps is at a temperature of about 90° C., 91° C., 92° C., 93° C., 94° C., or about 95° C. In embodiments, the plurality of denaturation steps is at a temperature of about 70° C.-85° C. In embodiments, the plurality of denaturation steps is at a temperature of about 70° C.-80° C. In embodiments, the plurality of denaturation steps is at a temperature of about 75° C.-80° C. In embodiments, the plurality of denaturation steps is at a temperature of about 70° C., 71° C., 72° C., 73° C., 74° C., 75° C., 76° C., 77° C., 78° C., 79° C., or about 80° C. In embodiments, the annealing/extension of the primer cycle is at a temperature of about 55° C., 56° C., 57° C., 58° C., 59° C., 60° C., 61° C., 62° C., 63° C., 64° C., or about 65° C.

In embodiments, forming a plurality of amplification products includes incubation in a denaturant. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO).

In embodiments, forming a plurality of amplification products includes a plurality of cycles of strand denaturation, primer hybridization, and primer extension. Although each cycle will include each of these three events (denaturation, hybridization, and extension), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start anew cycle with anew denaturation step, thereby amplifying the amplicons. Primer extension products from an earlier cycle may serve as templates for a later amplification cycle. In embodiments, the plurality of cycles is about 5 to about 50 cycles. In embodiments, the plurality of cycles is about 10 to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about 30 cycles.

In embodiments, forming a plurality of amplification products includes rolling circle amplification (RCA) (see, e.g., Lizardi et al., Nat. Genet. 19:225-232 (1998), which is incorporated herein by reference in its entirety). Several suitable RCA methods are known in the art. For example, RCA amplifies a circular polynucleotide (e.g., DNA) by polymerase extension of an amplification primer complementary to a portion of the template nucleic acid. This process generates copies of the circular polynucleotide template such that multiple complements of the template sequence arranged end to end in tandem are generated (i.e., a concatemer).

In embodiments, forming a plurality of amplification products includes exponential rolling circle amplification (eRCA). Exponential RCA is similar to the linear process except that it uses a second primer having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)).

In embodiments, forming a plurality of amplification products includes hyperbranched rolling circle amplification (HRCA). Hyperbranched RCA uses a second primer complementary to the first amplification product. This allows products to be replicated by a strand-displacement mechanism, which can yield a drastic amplification within an isothermal reaction (Lage et al., Genome Research 13:294-307 (2003), which is incorporated herein by reference in its entirety).

In embodiments, the method includes amplifying a template nucleic acid by extending an amplification primer with a strand-displacing polymerase for about 10 seconds to about 30 minutes. In embodiments, the method includes amplifying a template nucleic acid by extending an amplification primer with a strand-displacing polymerase for about 30 seconds to about 16 minutes. In embodiments, the method includes amplifying a template nucleic acid by extending an amplification primer with a strand-displacing polymerase for about 30 seconds to about 10 minutes. In embodiments, the method includes amplifying a template nucleic acid by extending an amplification primer with a strand-displacing polymerase for about 30 seconds to about 5 minutes. In embodiments, the method includes amplifying a template nucleic acid by extending an amplification primer with a strand-displacing polymerase for about 1 second to about 5 minutes. In embodiments, the method includes amplifying a template nucleic acid by extending an amplification primer with a strand-displacing polymerase for about 1 second to about 2 minutes.

In embodiments, the method includes amplifying a template nucleic acid by extending an amplification primer with a strand-displacing polymerase at a temperature of about 20° C. to about 50° C. In embodiments, the method includes amplifying a template nucleic acid by extending an amplification primer with a strand-displacing polymerase at a temperature of about 30° C. to about 50° C. In embodiments, the method includes amplifying a template nucleic acid by extending an amplification primer with a strand-displacing polymerase at a temperature of about 25° C. to about 45° C. In embodiments, the method includes amplifying a template nucleic acid by extending an amplification primer with a strand-displacing polymerase at a temperature of about 35° C. to about 45° C. In embodiments, the method includes amplifying a template nucleic acid by extending an amplification primer with a strand-displacing polymerase at a temperature of about 35° C. to about 42° C. In embodiments, the method includes amplifying a template nucleic acid by extending an amplification primer with a strand-displacing polymerase at a temperature of about 37° C. to about 40° C.

In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. A “phi polymerase” (or “D29 polymerase”) is a DNA polymerase from the D29 phage or from one of the related phages that, like D29, contain a terminal protein used in the initiation of DNA replication. For example, phi29 polymerases include the B103, GA-1, PZA, 015, BS32, M2Y (also known as M2), Nf, G1, Cp-1, PRD1, PZE, SFS, Cp-5, Cp-7, PR4, PR5, PR722, L17, 021, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase).

In embodiments, the double-stranded amplification product is provided in a clustered array. In embodiments, the clustered array includes a plurality of double-stranded amplification products localized to discrete sites on a solid support. In embodiments, the solid support is a bead. In embodiments, the solid support is substantially planar. In embodiments, the solid support is contained within a flow cell.

In embodiments, the sequencing includes sequencing by synthesis, sequencing by ligation, sequencing-by-binding, or pyrosequencing. In embodiments, generating a first sequencing read or a second sequencing read includes a sequencing by synthesis process. In embodiments, generating a first sequencing read or a second sequencing read includes sequencing-by-binding. As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a temary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

In embodiments, the method further includes generating a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated. In embodiments, the method further includes incorporating one or more unmodified dNTPs or one or more ddNTPs into the 3′ end of the extended sequencing primer.

In embodiments, sequencing includes (i) extending a sequencing primer by incorporating a labeled nucleotide, or labeled nucleotide analogue and (ii) detecting the label to generate a signal for each incorporated nucleotide or nucleotide analogue.

In embodiments, generating a sequencing read includes sequencing by synthesis, sequencing-by-binding, sequencing by ligation, or pyrosequencing.

In embodiments, the method includes sequencing the first and/or the second strand of a double-stranded amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057.026. Once such a modified nucleotide has been incorporated into the growing polynucleotide chain complementary to the region of the template being sequenced, there is no free 3-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

In embodiments, the methods of sequencing provided herein include aligning a portion of each sequencing read to a reference sequence. General methods for performing sequence alignments are known to those skilled in the art. Examples of suitable alignment algorithms, include but are not limited to the Needleman-Wunsch algorithm (see e.g. the EMBOSS Needle aligner available at www.ebi.ac.uk/Tools/psa/emboss_needle/, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/, optionally with default settings). Optimal alignment may be assessed using any suitable parameters of a chosen algorithm, including default parameters. In embodiments, the reference sequence is a reference genome. In embodiments, the methods of sequencing a template nucleic acid further include generating overlapping sequence reads and assembling them into a contiguous nucleotide sequence of a nucleic acid of interest. Assembly algorithms known in the art can align and merge overlapping sequence reads generated by methods of several embodiments herein to provide a contiguous sequence of a nucleic acid of interest. A person of ordinary skill in the art will understand which sequence assembly algorithms or sequence assemblers are suitable for a particular purpose taking into account the type and complexity of the nucleic acid of interest to be sequenced (e.g. genomic, PCR product, or plasmid), the number and/or length of deletion products or other overlapping regions generated, the type of sequencing methodology performed, the read lengths generated, whether assembly is de novo assembly of a previously unknown sequence or mapping assembly against a backbone sequence, etc. Furthermore, an appropriate data analysis tool will be selected based on the function desired, such as alignment of sequence reads, base-calling and/or polymorphism detection, de novo assembly, assembly from paired or unpaired reads, and genome browsing and annotation. In several embodiments, overlapping sequence reads can be assembled by sequence assemblers, including but not limited to ABySS, AMOS, Arachne WGA, CAP3, PCAP, Celera WGA Assembler/CABOG, CLC Genomics Workbench, CodonCode Aligner, Euler, Euler-sr, Forge, Geneious, MIRA, miraEST, NextGENe, Newbler, Phrap, TIGR Assembler, Sequencher, SeqMan NGen, SHARCGS, SSAKE, Staden gap4 package, VCAKE, Phusion assembler, Quality Value Guided SRA (QSRA), Velvet (algorithm), and the like. It will be understood that overlapping sequence reads can also be assembled into contigs or the full contiguous sequence of the nucleic acid of interest by available means of sequence alignment, computationally or manually, whether by pairwise alignment or multiple sequence alignment of overlapping sequence reads. Algorithms suited for short-read sequence data may be used in a variety of embodiments, including but not limited to Cross match, ELAND, Exonerate, MAQ, Mosaik, RMAP, SHRiMP, SOAP, SSAHA2, SXOligoSearch, ALLPATHS, Edena, Euler-SR, SHARCGS, SHRAP, SSAKE, VCAKE, Velvet, PyroBayes, PbShort, and ssahaSNP.

In embodiments, the methods of sequencing provided herein further include forming a consensus sequence for reads having the same UMI, or a portion thereof (e.g., a UMI sequence). In embodiments, the consensus sequence is obtained by comparing all sequencing reads aligning at a given nucleotide position (optionally, only among those reads identified as originating from the same sample polynucleotide molecule), and identifying the nucleotide at that position as the one shared by a majority of the aligned reads.

In embodiments, the methods of sequencing described herein further include computationally reconstructing sequences of a plurality of individual strands of original sample polynucleotides by removing UMI-derived sequences and joining sequences for adjacent portions of the sample polynucleotide. Reconstruction can be performed on individual reads, or on consensus sequences produced from those reads.

In embodiments, the methods of sequencing described herein further include aligning computationally reconstructed sequences.

Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer, can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated Ntimes to extend the primer by Nnucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes.

Use of the sequencing method outlined above is anon-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) to prevent further extension of the sequencing read product.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular nucleobase, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

In embodiments, the methods of sequencing a nucleic acid include extending a complementary polynucleotide (e.g., a primer) that is hybridized to the nucleic acid by incorporating a first nucleotide. In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof; and (d) a guanine nucleotide, or analog thereof.

In embodiments, the sequenced nucleotides include a scar remnant (e.g., an alkynyl moiety attached to the nucleobase). In embodiments, the nucleotides have the formula:

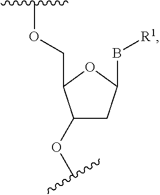

wherein B is a nucleobase, R¹ is the scar remnant, and “ [symbol] ” is the attachment point to the remainder of the sequenced strand polynucleotide.

In embodiments, B is a divalent nucleobase. In embodiments, B is

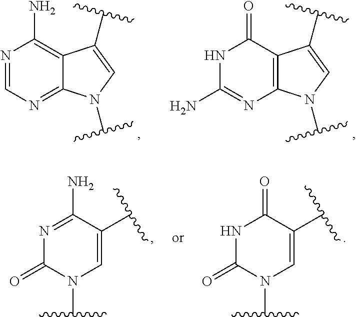 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

In embodiments, B is

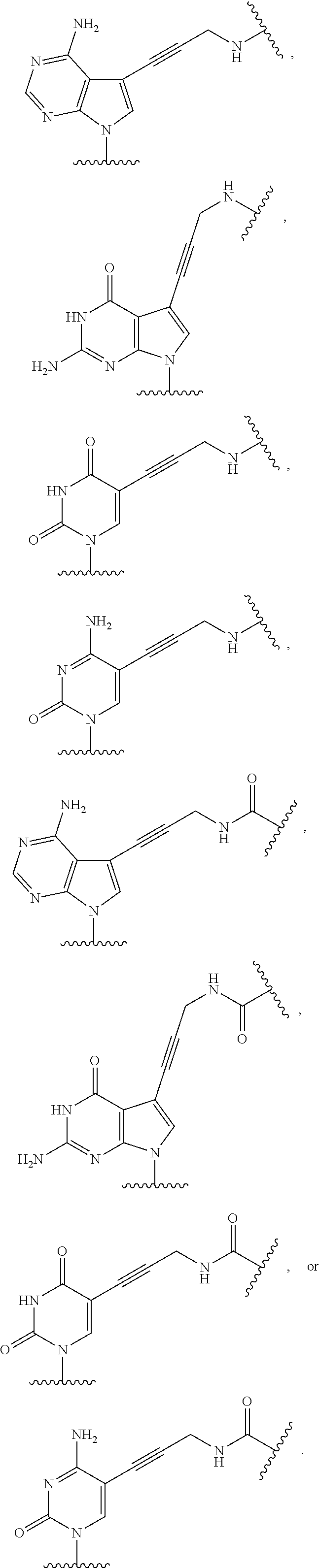

In embodiments, R¹ is hydrogen, —OH, —NH, a substituted or unsubstituted alkyl or substituted or unsubstituted heteroalkyl. In embodiments, R¹ is hydrogen. In embodiments, R¹ is —OH. In embodiments, R¹ is —NH. In embodiments, R¹ is a substituted or unsubstituted alkyl or substituted or unsubstituted heteroalkyl. In embodiments, R¹ is a substituted or unsubstituted alkenyl. In embodiments, R¹ is a substituted or unsubstituted alkynyl. In embodiments, R¹ is a substituted or unsubstituted heteroalkenyl. In embodiments, R¹ is a substituted or unsubstituted heteroalkynyl. In embodiments, R¹ is a substituted (e.g., substituted with a substituent group, size-limited substituent group, or lower substituent group) or unsubstituted alkyl or substituted (e.g., substituted with a substituent group, size-limited substituent group, or lower substituent group) or unsubstituted heteroalkyl. In embodiments, R¹ is substituted with an oxo or —OH. In embodiments, R¹ is substituted with an oxo and —OH.

In embodiments, R¹ is an oxo-substituted heteroalkyl (e.g., 2 to 10 membered heteroalkyl, 2 to 8 membered heteroalkyl, or 4 to 8 membered heteroalkyl). In embodiments, R¹ is an oxo-substituted heteroalkenyl (e.g., 2 to 10 membered heteroalkenyl, 2 to 8 membered heteroalkenyl, or 4 to 8 membered heteroalkenyl). In embodiments, R¹ is an oxo-substituted heteroalkynyl (e.g., 2 to 10 membered heteroalkynyl, 2 to 8 membered heteroalkynyl, or 4 to 8 membered heteroalkynyl). In embodiments, R¹ is an oxo-substituted 10 membered heteroalkynyl. In embodiments, R¹ is an oxo-substituted 9 membered heteroalkynyl. In embodiments, R¹ is an oxo-substituted 8 membered heteroalkynyl. In embodiments, R¹ is an oxo-substituted 7 membered heteroalkynyl. In embodiments, R¹ is an oxo-substituted 6 membered heteroalkynyl.

In embodiments, the one or more nucleotides including a scar remnant include a nucleobase having the formula

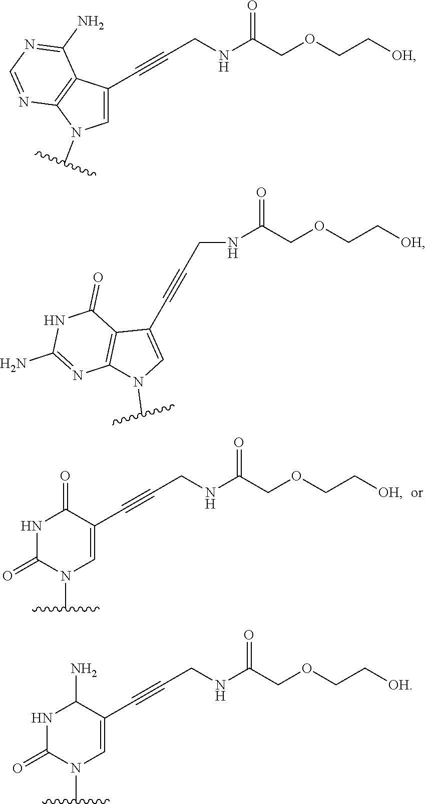 [SMILES: *n1cc(C#CCNC(=O)COCCO)c2c(N)ncnc21 ; *n1cc(C#CCNC(=O)COCCO)c2c(=O)[nH]c(N)nc21 ; *n1cc(C#CCNC(=O)COCCO)c(=O)[nH]c1=O ; *N1C=C(C#CCNC(=O)COCCO)C(N)NC1=O]

In embodiments, the one or more nucleotides including a scar remnant include a nucleobase having the formula

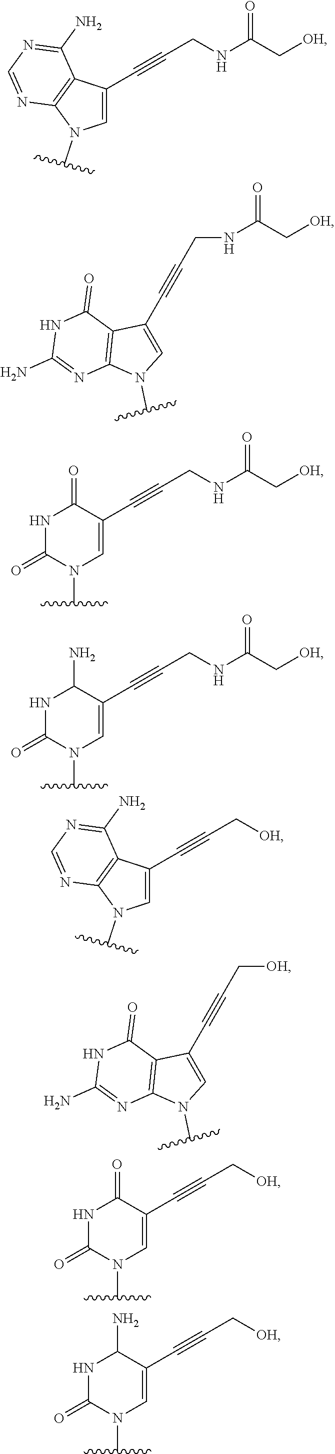

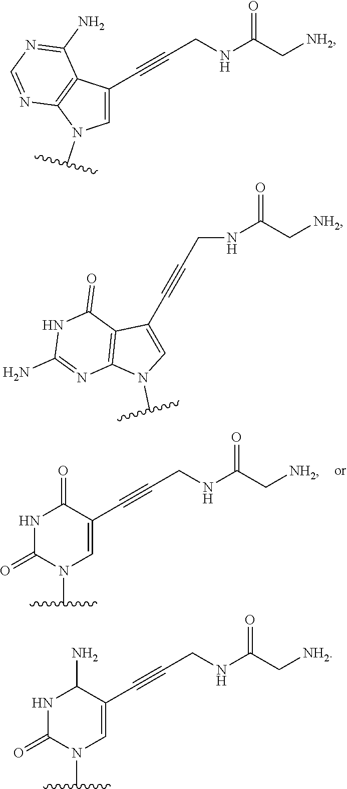 [SMILES: *n1cc(C#CCNC(=O)CN)c2c(N)ncnc21 ; *n1cc(C#CCNC(=O)CN)c2c(=O)[nH]c(N)nc21 ; *n1cc(C#CCNC(=O)CN)c(=O)[nH]c1=O ; *N1C=C(C#CCNC(=O)CN)C(N)NC1=O]

### III. Compositions & Kits

In an aspect is provided a kit. Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxvribonucleotides, ribonucleotides, labeled nucleotides, and/or modified nucleotides), buffers, salts, and/or labels (e.g., fluorophores).

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. The kit may also include a flow cell. In embodiments, kit includes the solid support and a flow cell carrier (e.g., a flow cell carrier as described in US 2021/0190668, which is incorporated herein by reference for all purposes).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

### EXAMPLES

### Example 1. Experimental Overview

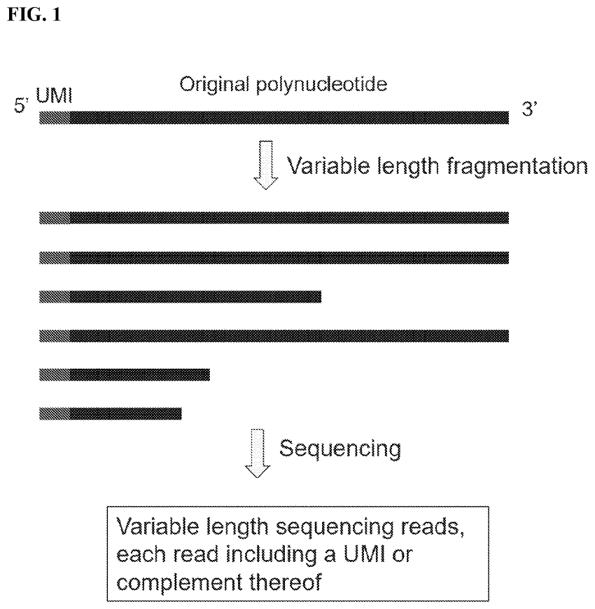

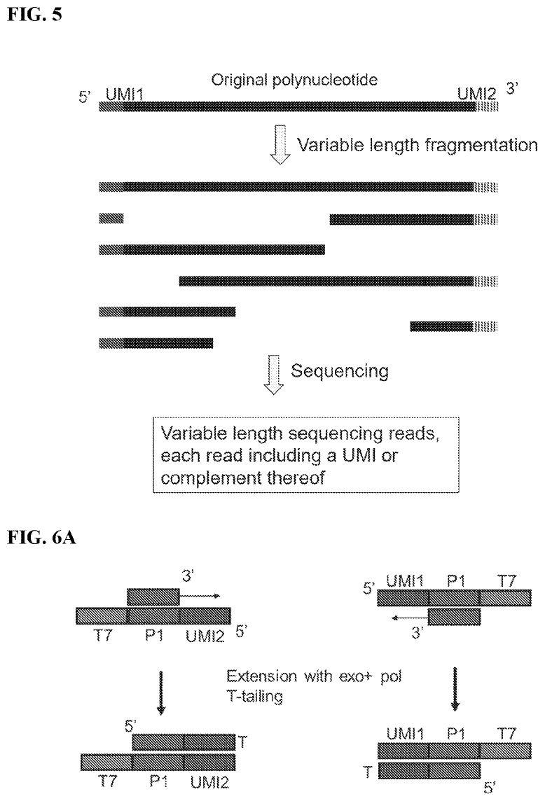

Conventional sequencing techniques (e.g., sequencing-by-synthesis, sequencing-by-binding, etc.) require de novo assembly of relatively short lengths of DNA (e.g., 35 to 300 base pairs), which makes resolving complex regions with mutations or repetitive sequences difficult. The application of those technologies to de novo genome assemblies is limited by short sequence read length, which, by previous methods, is insufficient to resolve complex genome structure and to produce consistent genome assembly. To address these limitations, researchers typically supplement short read sequencing data (e.g., short read sequencing data having an error rate of less than about 1.5%) with data from long read sequencers (e.g., read length 10kb, error rate 10-15%). Further, it is difficult to reliably obtain phasing data (i.e., which variants are on the same chromosome) or detect structural variants from short read data. Synthetic long-read technology, referred to as XR/T-Seq™, is known and can achieve greater than normal read lengths, for example, through the use of interspaced probes as described in U.S. Pat. No. 11,155,858, which is incorporated herein by reference in its entirety. Described herein are methods for achieving greater read lengths by random fragmentation of templates post-amplification, such that paired-read sequencing, for example, may be performed in combination with UMI matching and alignment (see, for example the illustrative outlines provided in FIG. 1 and FIG. 5).

Described herein are novel approaches for preparing libraries of polynucleotides that facilitate bioinformatic reconstruction to yield longer read lengths. For example, one method includes amplifying a sample polynucleotide that contains one or two unique molecular identifiers (UMIs), fragmenting the amplified polynucleotide to provide a distribution of polynucleotides having different lengths, attaching appropriate platform primer sequences and sequencing using common sequencing protocols. The nucleic acid fragments and their corresponding UMIs are grouped to reconstruct the original sample polynucleotide. In embodiments, the method includes ligating an adapter containing an RNA polymerase (RNAP) promoter sequence to a sample polynucleotide and subsequent RNA transcription with an RNA polymerase. Transcription using an RNA polymerase (e.g., T3, T5, or T7 RNA polymerase) generates thousands of copies of the template polynucleotide without the need for thermal cycling, effectively reducing the potential for the formation of PCR artifacts such as primer dimers and reducing bias. An additional advantage over PCR and/or exponential amplification is RNA transcription provides reduced amplification bias, which is a known issue with PCR, wherein some molecules get over-represented in the final library. Utilizing RNA linear amplification as described herein therefore reduces representation errors, especially when starting with low amounts of material. Finally, incorporating a cleavable site in an amplification product (e.g., a diol linkage, a restriction enzyme sequence, etc.) can, in embodiments, place an additional user burden and complicates the protocol. RNA molecules also provide an advantage in being able to be fragmented without the need for incorporation of a cleavable site during amplification.

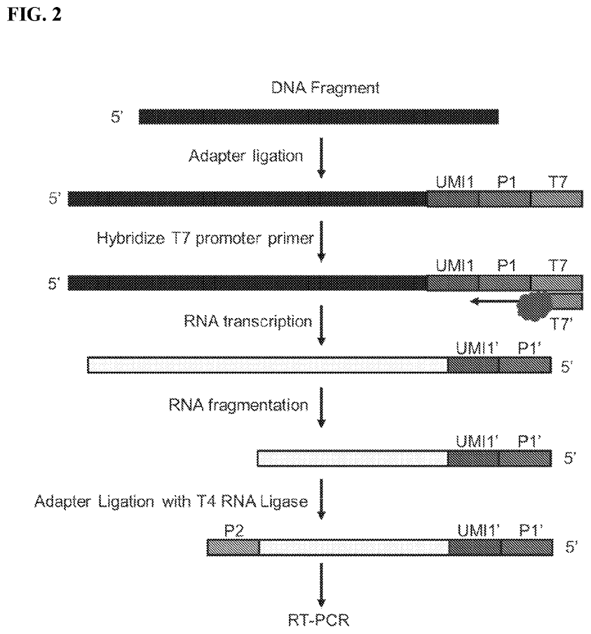

Linear amplification is performed using an RNA polymerase, for example the T7 RNA polymerase, as described herein. Any suitable RNA polymerase and corresponding promoter sequence may be used in the methods described herein. For example, to an isolated dsDNA sample polynucleotide (e.g., a dsDNA sample polynucleotide sequence containing a gene or pseudogene of interest), end-repair, and A-tailing is performed as described herein. A first adapter and a second adapter are ligated, wherein each adapter includes a UMI, a P1 primer binding sequence, and RNAP promoter sequence (e.g., a T7 RNAP promoter) (see, FIG. 2). The resulting adapter-target-adapter construct is then subjected to linear amplification by T7 RNA polymerase, generating a plurality of complementary RNA transcripts. In some embodiments, a second-strand synthesis step is performed (through single primer extension from the T7 RNAP promoter site) to create dsDNA prior to performing RNA linear amplification. Subsequently, RNA fragmentation is performed. Following RNA fragmentation, a single-stranded P2 adapter is ligated onto the 3′ end of the polynucleotide using, for example, T4 RNA ligase. Finally, reverse-transcriptase PCR (RT-PCR) is performed to generate a double-stranded product. For example, reverse transcription may first be performed with a primer specific to P2, and then PCR performed using primers specific to P1 and P2. Following RT-PCR, the template polynucleotides may be purified, amplified, and sequenced using methods known to those skilled in the art and as described herein. Alternate single-UMI approaches that do not employ an RNA intermediate are also described herein.

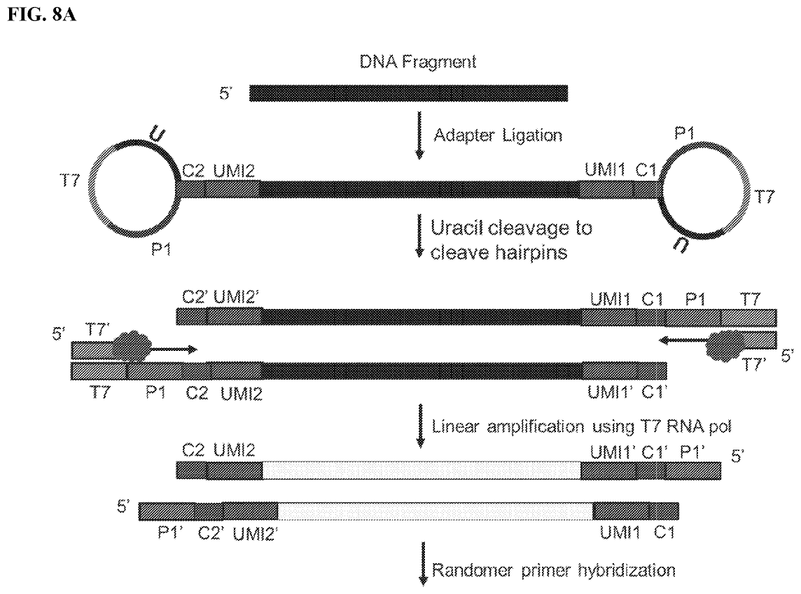

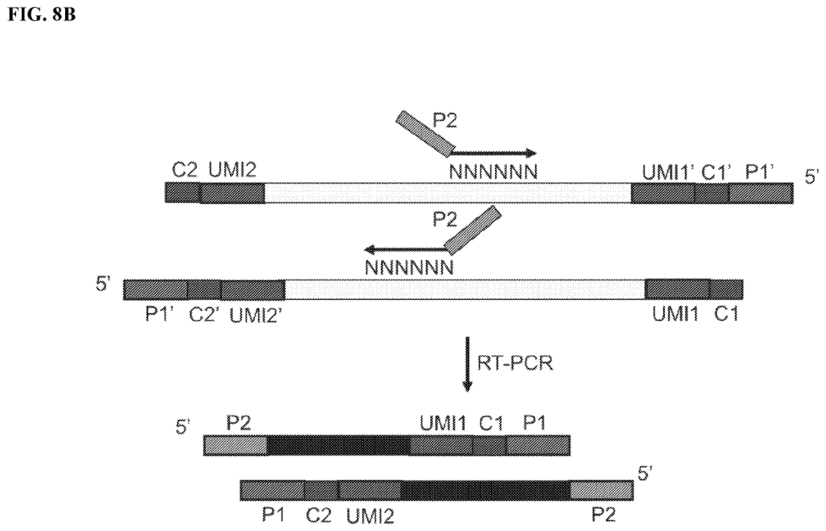

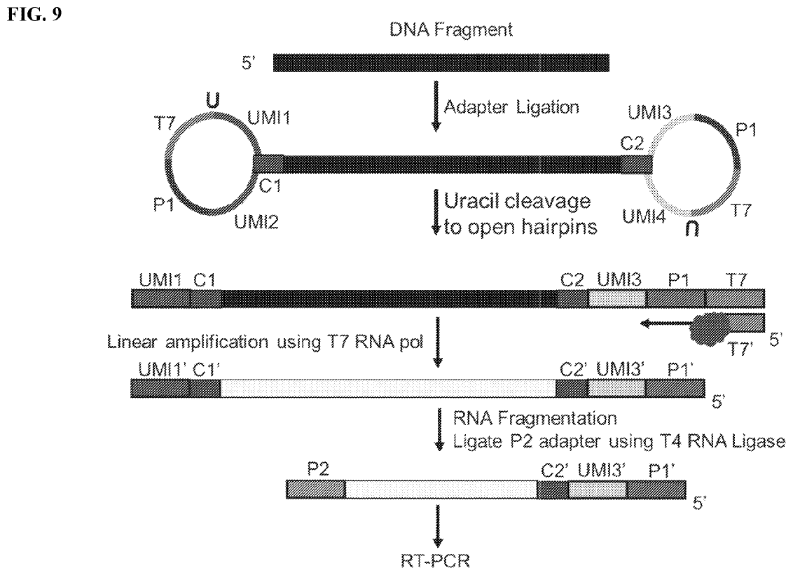

An alternative embodiment is also described herein which employs dual UMI, facilitating shorter paired-read mapping (see, FIG. 5). Prior to adapter ligation, adapters are generated in vitro as illustrated in FIG. 6A. In embodiments, a hairpin adapter including a cleavable site (e.g., a uracil), a RNAP promoter sequence (e.g., a T7 RNAP promoter), a P1 primer binding sequence, and a UMI is extended from the 3′ end to generate a complementary UMI sequence and T-tailed with a single T-base overhang (see, FIG. 7A). Following adapter ligation, (and cleavage in the example of a hairpin adapter), the resulting adapter-target-adapter construct is then subjected to linear amplification by T7 RNA polymerase, generating a plurality of complementary RNA transcripts (see, FIG. 6B). Following RNA fragmentation, single-stranded nucleic acid fragments including distinct UMI sequences are ligated with single-stranded P2 adapters using, for example, T4 RNA ligase. Alternatively, T4 RNA Ligase 2 Truncated KQ (T4 Rnl2tr R55K, K227Q mutant) may be used for ligation of the P2 adapter to the single-stranded nucleic acid fragments. In this case, the P2 adapter would first need to be 5′ adenylated, for example, with Mth RNA Ligase, prior to ligation. Finally, reverse-transcription (RT)-PCR is performed to generate double-stranded products. For example, RT may first be performed with a primer specific to P2, and then PCR performed using primers specific to P1 and P2. An alternate embodiment of a dual-UMI containing construct is presented in FIG. 9, wherein the template polynucleotide is ligated with hairpin adapters containing two UMI sequences in the loop region, separated by a cleavable site. In another embodiment, randomer primers are hybridized to the linear amplification RNA products, wherein the randomer primers include a P2 adapter sequence on the 5′ end (FIGS. 8A-8B). Random hybridization of the randomer primers, followed by RT and PCR as described above, results in variable-sized dsDNA polynucleotide fragments. The nucleic acid fragments are then purified, amplified, and sequenced using methods known to those skilled in the art and as described herein. This method is advantageous in that it bypasses the need to perform a separate RNA fragmentation step (e.g., as with the other RNA-intermediate approaches described herein). Additionally, the P2 adapter ligation step is no longer necessary as the randomer primer introduces this sequence during RT-PCR. Alternate dual-UMI embodiments that do not include RNA intermediates are also described herein.

Inheritance patterns of genetic variation in complex traits may be influenced by interactions among multiple genes and alleles across long distances. Examination of phased variants are critical for a greater understanding of the genetic basis of complex phenotypes (see, for example, Snyder, M. W., Adey, A., Kitzman, J. O. & Shendure, J. “Haplotype-resolved genome sequencing: experimental methods and applications” Nat. Rev. Genet. 16, 344-358 (2015)). Additionally, resolving long-range information at the molecular level within complex samples, e.g., cancer samples, is essential to assemble and phase variants of subpopulations of cells, as genetic drivers and important diagnostic biomarkers in cancers and other diseases (see, for example, Moncunill, V. et al. Comprehensive characterization of complex structural variations in cancer by directly comparing genome sequence reads. Nat. Biotechnol. 32, 1106-1112 (2014)). Experiments herein demonstrate that long-ranged nucleic acid sequencing can be performed in one physical compartment, offering efficiencies relative to methods known in the art.

### Example 2. T-Cell and B-Cell Receptor Repertoire Sequencing

Traditional sequencing-by-synthesis (SBS) methodologies employ serial incorporation and detection of labeled nucleotide analogues. For example, high-throughput SBS technology (see, for example, Bentley D R, et al. Nature, 2008, 456, 53-59) uses cleavable fluorescent nucleotide reversible terminator (NRT) sequencing chemistry (see, for example, see U.S. Pat. No. 6,664,079; or Ju et al. Proc. Natl. Acad. Sci. USA, 2006, 103, 19635-19640). These cleavable fluorescent NRTs were designed based on the following rationale: each of the four nucleotides (A, C, G, T, and/or U) is modified by attaching a unique cleavable fluorophore to the specific location of the nucleobase and capping the 3′-OH group of the nucleotide sugar with a small reversible moiety (also referred to herein as a reversible terminator) so that they are still recognized by DNA polymerase as substrates. The reversible terminator temporarily halts the polymerase reaction after nucleotide incorporation while the fluorophore signal is detected. After incorporation and signal detection, the fluorophore and the reversible terminator is cleaved to resume the polymerase reaction in the next cycle.

Applications of NGS to genomes, transcriptomes, and epigenomes may be applied to immune profiling. The functions of immune cells such as B- and T-cells are predicated on the recognition through specialized receptors of specific targets (antigens) in pathogens. There are approximately 10¹⁰ to 10¹¹ B-cells and 10¹¹ T-cells in a human adult (see, for example, Ganusov V V, De Boer R J. Trends Immunol. 2007; 28(12):514-8; and Bains I, Antia R, Callard R, Yates A J. Blood. 2009; 113(22):5480-5487).

Immune cells are critical components of adaptive immunity and directly bind to pathogens through antigen-binding regions present on the cells. Within lymphoid organs (e.g., bone marrow for B cells and the thymus for T cells) the gene segments variable (V), joining (J), and diversity (D) rearrange to produce a novel amino acid sequence in the antigen-binding regions that allow for the recognition of antigens from a range of pathogens (e.g., bacteria, viruses, parasites, and worms) as well as antigens arising from cancer cells. The large number of possible V-D-J segments, combined with additional (junctional) diversity, lead to a theoretical diversity of >10¹⁴, which is further increased during adaptive immune responses. Overall, the result is that each B- and T-cell expresses a highly variable receptor, whose sequence is the outcome of both germline diversity and somatic recombination. Somatic recombination is a process that creates new combinations of V, D and J segments via a complicated mechanism that involves gene excision and alternative splicing. These antibodies also contain a constant (C) region, which confers the isotype to the antibody. In most mammals, there are five antibody isotypes: IgA, IgD, IgE, IgG, and IgM. For example, each antibody in the IgA isotype shares the same constant region. Characterization of an individual's immune repertoire (i.e., the global profile of which immune cell receptors are present in an individual), requires full length sequencing of the recombined VDJ region, which is difficult to determine with short read sequencing data. Thus, obtaining long-range sequence data is incredibly insightful to gain insights into the adaptive immune response in healthy individuals and in those with a wide range of diseases.

For example, while parts of the B-cell immunoglobulin receptor (BCR) can be traced back to segments encoded in the germline (i.e., the V, D and J segments), the set of segments used by each receptor is something that needs to be determined as it is coded in a highly repetitive region of the genome (see, for example, Yaari G, Kleinstein S H. Practical guidelines for B-cell receptor repertoire sequencing analysis. Genome Med. 2015; 7:121. (2015)). Additionally, there are no pre-existing full-length templates to align the sequencing reads.

Commercially available next-generation sequencing (NGS) technologies typically require library preparation, whereby a pair of specific adapter sequences are ligated to the ends of DNA polynucleotides in order to enable sequencing by the instrument. Typically, preparation of a nucleic acid library involves 5 steps: DNA fragmentation, polishing, adapter ligation, size selection, and library amplification. There are two starting materials that can serve as the initial template to sequence immunoglobulin (Ig) repertoires-genomic DNA (gDNA) and mRNA. Use of gDNA as a template has particular advantages over mRNA when alternative splicing does not take place, namely using mRNA requires an additional step to convert RNA to DNA via reverse transcription. However, within a cell, there is a single copy of gDNA, whereas the quantity of mRNA varies by orders of magnitude. Regardless, either gDNA or mRNA can serve as input.

### Single UMI-Based Amplification Methods

A novel approach is described herein which involves ligation of an adapter including an RNAP promoter sequence and subsequent RNA transcription with an RNA polymerase. Performing a first linear amplification step via an RNA intermediate has several advantageous over an entirely DNA-based amplification protocol, for example, boosting the signal of high-quality molecules by >1000× prior to doing traditional PCR. Additionally, since RNA linear amplification always amplifies from the original template polynucleotide, chimeric structures, if formed during amplification, are not propagated. This results in a lower chance of errors in the reassembled long molecules and greater efficiency. The T7 RNA polymerase, for example, can make thousands of copies of a template polynucleotide without the need for thermal cycling, effectively reducing the potential for the formation of PCR artifacts such as chimeric structures and reducing bias. Another advantage over PCR/exponential amplification is lower amplification bias, which is a known issue with PCR, where some molecules get over-represented in the final library. Beginning with an RNA linear amplification step may therefore reduce errors, especially when starting with low amounts of material. RNA molecules also provide an advantage in being able to be fragmented without the need for incorporation of a cleavable site during amplification.

T7 RNA polymerase (RNAP) has high specificity for its promoter, the requirement of no additional transcription factors, and high fidelity of initiation from a specific site in the promoter. Bacteriophage polymerases generally have a 23-nucleotide promoter that overlaps the site of transcription initiation by six nucleotides (−17 to +6) (Padmanabhan R and Miller D. bioRxiv 2019; 619395). The full T7 promoter consists of the sequence TAATACGACTCACTATAGGGAGA (SEQ ID NO: 3). The minimal T7 RNAP promoter sequence able to support de novo initiation at a recessed 3′ end in vivo is CACTATAGGG (SEQ ID NO: 4). In embodiments of the method herein, the length of the T7 RNAP promoter sequence used for linear amplification may be tailored to suit the requirements of a specific adapter ligation protocol.

Method: T7 RNA Polymerase Amplification and RNA Fragmentation with Single UMI

To an isolated dsDNA sample polynucleotide (e.g., a sample polynucleotide sequence containing a gene or pseudogene), end-repair, and A-tailing is performed as described herein. A first adapter and a second adapter are thereafter ligated, wherein each of the first adapter and second adapter include a UMI, P1 primer binding sequence, and RNAP promoter (e.g., a T7 RNAP promoter) (see, FIG. 2). The resulting adapter-target-adapter construct is then diluted to a suitable concentration and linearly amplified using a T7 RNA polymerase, generating a plurality of complementary RNA transcripts. In some embodiments, a second-strand synthesis step is performed (through single primer extension from the T7 RNAP promoter site) to create dsDNA prior to performing RNA linear amplification. Subsequently, RNA fragmentation is performed. Various methods for RNA fragmentation are known in the art, including the use of alkaline hydrolysis or metal ion-based cleavage (magnesium or zinc ions) (Marchand V et al. Nucleic Acids Res. 2016; 44(16): e135). Commercial solutions for metal ion-based cleavage are available, including the NEBNext® Magnesium RNA Fragmentation Module (NEB Catalog #E6150S). The size of the RNA fragments generated during metal ion-based cleavage can be tuned based on incubation times, and stopped with a metal-chelating solution, for example, an EDTA solution.

Following RNA fragmentation, a single-stranded P2 adapter is ligated onto the 3′ end of the polynucleotide fragment using, for example, T4 RNA ligase. Alternatively, T4 RNA Ligase 2 Truncated KQ (T4 Rnl2tr R55K, K227Q) may be used for ligation of the P2 adapter to the single-stranded fragments. In this case, the P2 adapter would first need to be 5′ adenylated, for example, with Mth RNA Ligase, prior to ligation. Finally, reverse-transcriptase PCR (RT-PCR) is performed to generate a double-stranded DNA polynucleotide. For example, RT may first be performed with a primer specific to P2, and then PCR performed using primers specific to P1 and P2. Following RT-PCR, the nucleic acid templates may be purified, amplified, and sequenced using methods known to those skilled in the art and as described herein.

Method: Random Cleavable Site Incorporation with Single UMI

To an isolated polynucleotide (e.g., B-cell immunoglobulin receptor) sample, the polynucleotide is fragmented using methods known in the art. Fragmentation of polynucleotides can be achieved by enzymatic digestion or physical methods (e.g., sonication, nebulization, or hydrodynamic shearing). Enzymatic digestion produces DNA ends that can be efficiently polished and ligated to adapter sequences. However, it is difficult to control the enzymatic reaction and produce nucleic acid fragments of predictable length. In addition, enzymatic fragmentation is frequently base-specific thus introducing representation bias into the sequence analysis. Alternatively, physical methods to fragment DNA are random and DNA size distribution can be more easily controlled, but DNA ends produced by physical fragmentation are often damaged and a conventional polishing reaction may be insufficient to generate ample ligation-compatible ends. In embodiments, the input polynucleotide is fragmented into about 1,000 to about 2,000 base pair nucleic acid fragments and optionally polished. Typical polishing mixtures contain T4 DNA polymerase and T4 polynucleotide kinase. These enzymes excise 3′ overhangs, fill in 3′ recessed ends, and remove any potentially damaged nucleotides thereby generating blunt ends on the nucleic acid fragments. The T4 polynucleotide kinase used in the polishing mix adds a phosphate to the 5′ ends of DNA fragments that can be lacking such, thus making them ligation-compatible to NGS adapters.

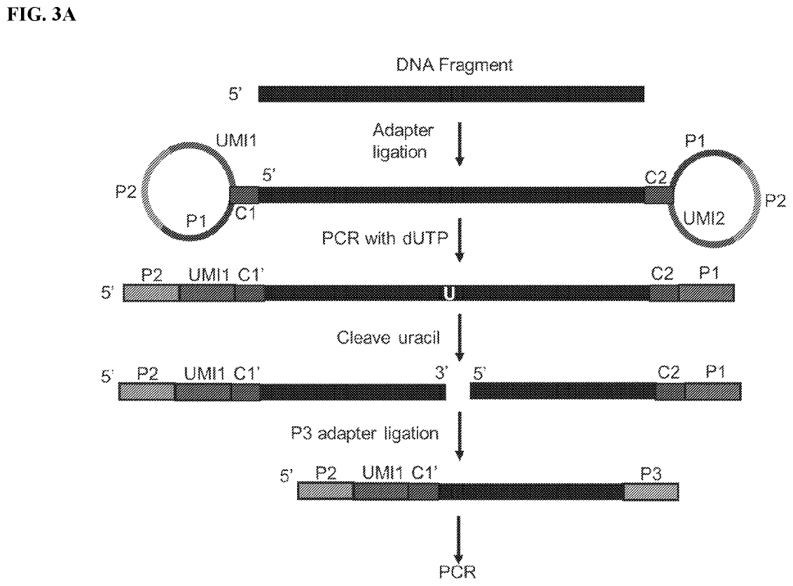

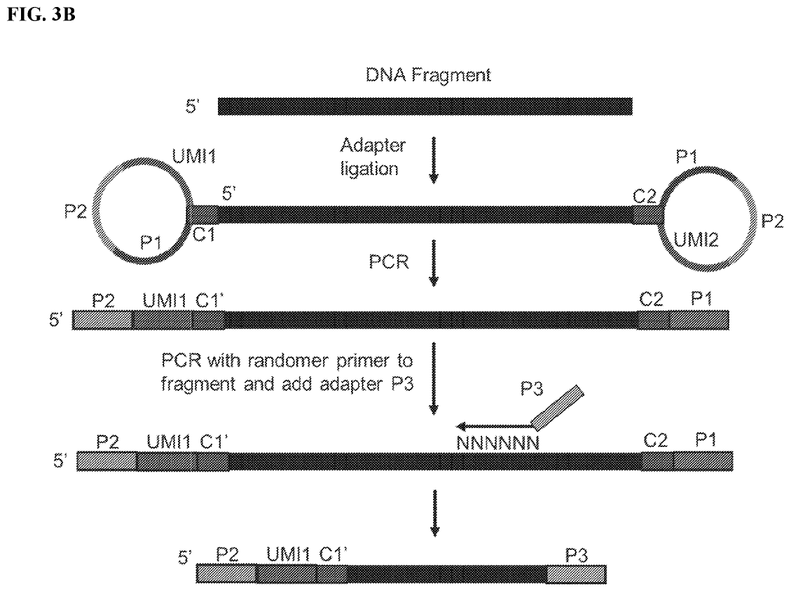

Prior to ligation, adenylation of repaired nucleic acids using a polymerase which lacks 3′-5′ exonuclease activity is typically performed in order to minimize chimera formation and adapter-adapter (dimer) ligation products. In these methods, single 3′ A-overhang DNA fragments are ligated to single 5′ T-overhang adapters, whereas A-overhang fragments and T-overhang adapters have incompatible cohesive ends for self-ligation. A ligation reaction between a first adapter, a second adapter, and the DNA fragments is then performed using a suitable ligase enzyme (e.g., T4 DNA ligase) which joins each adapter to each DNA fragment, one at either end, to form adapter-target-adapter constructs (see, FIG. 3A). The products of this reaction can be purified from leftover unligated adapters that by a number of means (e.g., NucleoMag NGS Clean-up and Size Select kit, Solid Phase Reversible Immobilization (SPRI) bead methods such as AMPureXP beads, PCRclean-dx kit, III Axygen AxyPrep FragmentSelect-I Kit), including size-inclusion chromatography, preferably by electrophoresis through an agarose gel slab followed by excision of a portion of the agarose that contains the DNA greater in size that the size of the adapter.

In some embodiments, each of the first adapter and second adapter includes about 6 to about 20 random nucleotides on the 3′ end. Such random sequences may be referred to as molecular barcodes or unique molecular identifiers (UMI). In embodiments of the methods described herein, synthetic long reads are constructed by grouping together UMIs based on direct or indirect co-occurrence in the library, and then assembling the reads back into the original full-length molecule. In embodiments, synthetic long reads are constructed by grouping together UMIs based on direct or indirect co-occurrence in the library, and then assembling the reads back into the original full-length molecule, wherein the grouping is performed by a computer and the computer outputs the result of the grouping. In embodiments, the length of the UMI is optimized based on the total number of insertions sites (number of targeted molecules X number of insertion locations) to reduce the incorporation of two of the same UMIs in different molecules, while maximizing the amount of sequence in the read that is from the target molecule. Rare instances where the same UMI is observed in two different molecules can be addressed bioinformatically. Aside from forming the backbone for long read alignment, the introduction of UMIs into sequencing libraries prior to target amplification has been shown to dramatically increase the sensitivity for rare mutations and enable absolute read counting. In addition to UMIs, each adapter contains two primer binding sites, labeled as P1 and P2 in FIG. 3A.

The adapter-target-adapter construct may be amplified using methods known to those skilled in the art (e.g., standard PCR amplification or rolling circle amplification). Amplification in the presence of a random cleave point is useful to generate a fragmented sample of polynucleotides. In some embodiments, amplification occurs in the presence of a suitable concentration of dUTP such that the adapter-target-adapter construct is copied with an incorporation rate of about 1 dUTP per extension (see, FIG. 3A). Subsequently, cleavage and degradation at dU sites may be achieved using, for example, uracil DNA glycosylase and endonuclease VIII (USER™, NEB, Ipswich, Mass.) as described in US 2003/0194736 or under other appropriate cleaving conditions known in the art. In embodiments, the adapter-target-adapter construct is amplified in the presence of a suitable concentration of reversibly terminated nucleotide triphosphates (modified NTPs) such that the adapter-target-adapter construct is copied with an incorporation rate of a single modified NTP per extended strand. Upon incorporation of a reversibly terminated NTP, extension of the polynucleotide strand is terminated and may not be further extended.

Following cleavage at the dU sites (or chain termination by reversibly terminated NTP incorporation) the resulting adapter-construct-adapter nucleic acid fragments may be isolated. Isolation and purification of the nucleic acid fragments can be accomplished, for example, but pulling-down a biotin-labeled end of the nucleic acid fragment with streptavidin-coated solid-support, or by hybridizing a solid-support-conjugated oligonucleotide that is complementary to the P1 or P2 adapter sequences. For reversibly terminated extension products, the terminators are cleaved using suitable means for a given terminator to generate a 3′-OH end prior to adapter ligation. Cleaved double-stranded DNA is then end-repaired and A-tailed as described supra. Following A-tailing, additional adapters or primers may be added using conventional means to permit platform specific sequences and/or to provide a binding site for sequencing primers. Following adapter ligation, the nucleic acid templates may be purified, amplified, and sequenced using methods known to those skilled in the art and as described herein.

In an alternate embodiment of the method described supra, following adapter ligation (e.g., a first adapter and second adapter as shown in FIG. 3A), PCR amplification is performed on the adapter-target-adapter construct using primers complementary to the P1 and P2 regions, for example, as shown in FIG. 3A. Subsequently, PCR is performed using a random base primer attached to an adapter containing a P3 primer binding site, as illustrated in FIG. 3B. The random base primer will randomly hybridize to the target construct, thereby facilitating the amplification of truncated nucleic acid fragments that contain a UMI and the P3 adapter. Following PCR, the nucleic acid templates may be purified, amplified, and sequenced using methods known to those skilled in the art and as described herein.

Method: Linear Amplification and Random Cleavage with Single UMI

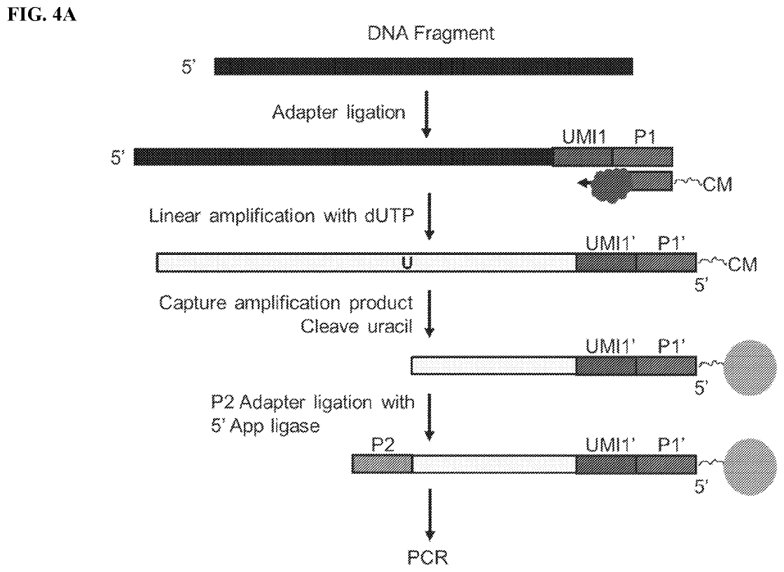

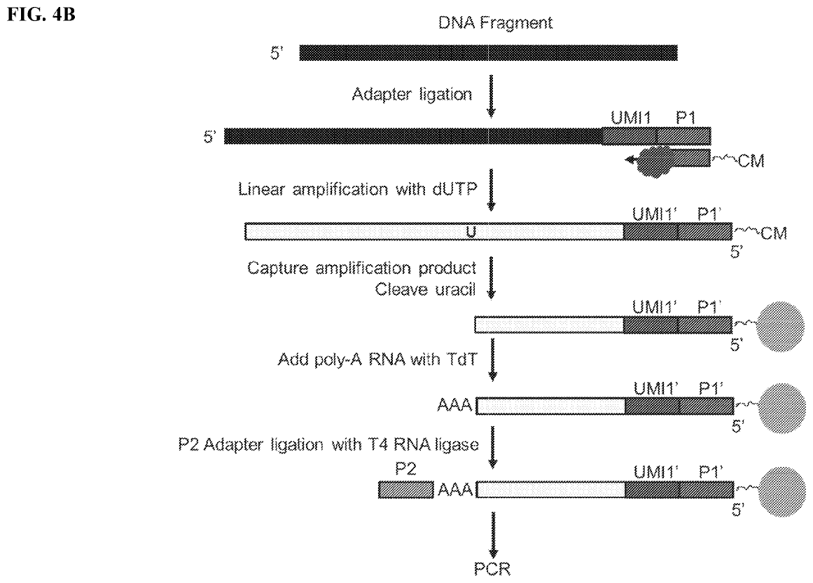

In an alternate embodiment of the methods described supra, following end-repair and A-tailing of a target polynucleotide fragment, adapters are ligated onto both ends of the target dsDNA, resulting in an adapter-target-adapter construct as shown in FIG. 4A. The adapter includes a primer binding sequence (e.g., P1), a UMI (e.g., UMI1), and optionally, a duplexed constant region (e.g., C1). Following adapter ligation, the adapter-target-adapter constructs are diluted to ensure sufficient representation per molecule in subsequent amplification steps. Linear amplification is then performed, for example, with a biotinylated primer (or a primer containing another suitable capture moiety) complementary to the P1 sequence with a reaction mixture containing a concentration of dUTP nucleotides such that a single uracil is incorporated per extended strand. Following amplification, the biotinylated extension product is pulled down using, for example, a streptavidin-coated solid support. The isolated extension product is then cleaved at the cleavable site, for example, uracil cleavage by uracil DNA glycosylase and an abasic site-specific endonuclease.

Following uracil cleavage, the free 3′ end may be ligated with a single-stranded 5′ adenylated adapter using, for example, a 5′ App DNA/RNA ligase, as shown in FIG. 4A. Alternatively, following uracil cleavage of the extension product, a short stretch (e.g., 3 to 5 bases) of poly-A RNA is added with terminal deoxyribonucleotidyl transferase (TdT), as shown in FIG. 4B. The poly-A RNA facilitates the ligation of a single-stranded DNA P2 adapter through the use of, for example, T4 RNA ligase. Additional details and methodologies for ssDNA ligation are known in the art (e.g., Miura F et al. Nucleic Acids Res. 2019; 47(15): e85, which describes TdT-mediated ssDNA ligation, and Gansauge M T et al. Nucleic Acids Res. 2017; 45(10): e79, which describes ssDNA ligation with T4 DNA ligase utilizing a splinter oligonucleotide, each of which is hereby incorporated by reference in their entirety). Following ligation of the single-stranded adapter onto the single-stranded fragmented product, an additional PCR amplification step is performed using primers complementary to the P1 and P2 regions to generate a double-stranded product. Following PCR, the nucleic acid templates may be purified, amplified, and sequenced using methods known to those skilled in the art and as described herein.

### Dual UMI-Based Amplification Methods

Method: T7 RNA Polymerase Amplification and RNA Fragmentation with Dual UMIs

An alternative embodiment of the RNA-based amplification and fragmentation method described supra is now described which employs dual UMI, facilitating shorter paired-end read mapping (see, FIG. 5). Prior to adapter ligation, adapters are generated in vitro as illustrated in FIG. 6A. Briefly, an adapter template including a RNAP promoter, e.g., a T7 RNAP promoter), a P1 primer binding sequence, and a UMI are hybridized with a primer complementary to an internal region, followed by extension with an exonuclease-defective polymerase to generate a complementary UMI sequence and T-tailed with a single T-base overhang. The adapter may also be a hairpin adapter. In some embodiments, a hairpin adapter including a cleavable site (e.g., a uracil), a RNAP promoter (e.g., a T7 RNAP promoter), a P1 primer binding sequence, and a UMI is extended from the 3′ end to generate a complementary UMI sequence and T-tailed with a single T-base overhang (see, FIG. 7A). Alternatively, a hairpin adapter containing additional sequence 5′ of the UMI sequence is annealed with a single T-base overhand-containing linking polynucleotide sequence, followed by extension of the hairpin oligo 3′ end to generate a complementary UMI sequence (see, FIG. 7B). The complementary UMI sequence is then ligated onto the linking oligo using a DNA ligase (e.g., T4 DNA ligase).

To an isolated dsDNA sample polynucleotide (e.g., a nucleic acid sequence containing a gene or pseudogene), end-repair and A-tailing is performed as described herein. The first adapter and a second adapter generated in FIG. 6A are thereafter ligated (see, FIG. 6B). For the hairpin ligated nucleic acid, cleavage of the cleavable site (e.g., USER enzyme cleavage of uracil) is performed to cleave the hairpins (see, FIG. 8A). The resulting adapter-target-adapter construct is then diluted to an optimal concentration and subjected to linear amplification by T7 RNA polymerase, generating a plurality of complementary RNA transcripts (see, FIG. 6B). In some embodiments, a second-strand synthesis step is performed (through single primer extension from the T7 RNAP promoter site) to create dsDNA prior to performing RNA linear amplification. Prior to proceeding with RNA fragmentation, an aliquot fraction of full-length RNA product may be retained, and subsequently ligated with a single-stranded P2 adapter (not shown), followed by RT-PCR. For example, RT may first be performed with a primer specific to P2, and then PCR performed using primers specific to P1 and P2. This full-length product may be spiked in during sequencing to act as a reference sequence for the shorter paired-end reads that will be generated from the fragmented templates. Preferentially, RNA fragmentation is optimized such that a small fraction of the starting RNA is not cleaved, thereby including some full-length RNA product for downstream sequencing. Following RT-PCR as described above, the nucleic acid templates may be purified, amplified, and sequenced using methods known to those skilled in the art and as described herein. In embodiments, the full-length RNA is about 500, 1000, 1500, 2000, 2500, 3000, 3500, 4000, 4500, or 5000 nucleotides in length. In embodiments, the full-length RNA is about 500, 1000, 2000, 5000, 7000, or 10000 nucleotides in length.

Subsequently, RNA fragmentation is performed as described supra. Following RNA fragmentation, single-stranded fragments including distinct UMI sequences are ligated with single-stranded P2 adapters using, for example, T4 RNA ligase (see, FIG. 6B). Alternatively, T4 RNA Ligase 2 Truncated KQ (T4 Rnl2tr R55K, K227Q mutant) may be used for ligation of the P2 adapter to the single-stranded nucleic acid fragments. In this case, the P2 adapter would first need to be 5′ adenylated, for example, with Mth RNA Ligase, prior to ligation. Finally, reverse-transcription (RT)-PCR is performed to generate double-stranded products. For example, RT may first be performed with a primer specific to P2, and then PCR performed using primers specific to P1 and P2. Following RT-PCR as described above, the nucleic acid templates may be purified, amplified, and sequenced using methods known to those skilled in the art and as described herein.

An alternate embodiment of a dual-UMI containing construct is presented in FIG. 9, wherein the template nucleic acid is ligated with hairpin adapters containing two UMI sequences in the loop region, separated by a cleavable site. Following end-repair and A-tailing of a dsDNA sample polynucleotide prior, adapter ligation (e.g., hairpin adapter ligation) is performed. Each hairpin adapter includes two UMI sequences (e.g., UMI1 and UMI2), a cleavable site (e.g., one or more uracils), a primer binding sequence (e.g., P1) and a duplexed constant region (e.g., C1/C2) adjacent to the UMIs. Following ligation, the hairpins are cleaved and linear amplification using RNAP (e.g., T7 RNAP) and fragmentation is performed as described herein. In some embodiments, a second-strand synthesis step is performed (through single primer extension from the T7 RNAP promoter site) to create dsDNA prior to performing RNA linear amplification. These double-stranded products include nucleic acid fragments spanning the entire length of the reference target polynucleotide, represented by two distinct UMIs. Uracil cleavage is then performed to cleave the hairpin adapters, followed by linear amplification using T7 RNA polymerase (illustrated as a cloud-shaped object). FIG. 9 further illustrates the step of RNA transcription using T7 RNA polymerase prior to RNA fragmentation. Following RNA transcription, the RNA product is fragmented (e.g., using a Mg-based fragmentation solution), and a P2 primer binding site-containing adapter is ligated using T4 RNA ligase. RT-PCR is then performed to generate dsDNA (i.e., cDNA) template polynucleotides with distinct UMIs. Following RT-PCR as described above, the nucleic acid templates may be purified, amplified, and sequenced using methods known to those skilled in the art and as described herein.

### Method: T7 RNA Polymerase Amplification and Randomer Primer Fragmentation

An alternative embodiment of the RNA-based amplification and fragmentation method described supra employing dual UMIs, is described herein. FIG. 8A illustrates a dsDNA sample polynucleotide with ligated adapters on each end, generated as described supra and in FIG. 7A. dsDNA sample polynucleotides were phosphorylated and A-tailed suing the NEBNext® Ultra II kit. Prior to ligation, adapters were T-tailed and phosphorylated, and ligation reaction mixture was added, including T4 DNA ligase. Exonuclease III was added to the ligation product to remove undesired ligation products, and the reaction was purified using a Zyme Oligo Clean and Concentrator. Hairpin adapter ligation on both ends of the sample polynucleotide was confirmed by gel electrophoresis. Following ligation of two hairpin adapters (hairpin adapters as shown in FIG. 7A), the hairpins were cleaved open via uracil cleavage by USER enzyme. FIG. 8A illustrates the step of cleaving open the hairpin (e.g., uracil cleavage by USER enzyme mix) to generate two non-covalently linked strands. Following hairpin cleavage, linear RNA amplification was performed using T7 RNA polymerase (T7 RNAP), as shown in FIG. 8A. We observed the expected linear RNA amplification product, as confirmed by gel electrophoresis, of about 1000 bp in size. Following 18 hours of linear RNA amplification by T7 RNAP, approximately 40 pmol of RNA was generated, from 1 μL of input template.

It has been reported that sequences from position +4 to +8 downstream of the transcription start site affect T7 promoter activity over a 5-fold range (see, e.g., Conrad T et al. Comms. Biol. 2020; 3:439, which is incorporated herein by reference in its entirety). For example, AT-rich sequences such as ATAAT (SEQ ID NO: 5), ATTAT (SEQ ID NO: 6), AAATA (SEQ ID NO: 7) and AATTC (SEQ ID NO: 8) located from position +4 to +8 downstream of the transcription start site, or flanking the T7 promoter, produced higher levels of linear RNA amplification than without the additional AT-rich sequences.

In some embodiments, prior to performing RNA transcription, a dsDNA extension product is generated by hybridization of a primer to the T7 RNA promoter and performing a primer extension (not shown). FIG. 8B illustrate the steps of randomer primer (e.g., DNA oligonucleotides including a template hybridization region) hybridization, wherein the randomer primer includes a template hybridization region Rn and a P2 primer binding sequence, to the linear amplification RNA polynucleotide products. In some embodiments, the randomer hybridization region Rn includes a random hexamer sequence (e.g., a template hybridization sequence). Following hybridization, RT-PCR was performed as described above to generate randomly-sized dsDNA template polynucleotides with distinct UMIs. We observed a range of products between 125 bp to 1000 bp by gel electrophoresis, as expected, representing the entire length of the template. This method is advantageous in that it bypasses the need to perform a separate RNA fragmentation step (e.g., a metal ion-based cleavage step as with the other RNA-intermediate approaches described herein). Additionally, the P2 adapter ligation step is no longer necessary as the randomer primer introduces this sequence.

Another consideration when implementing a long-read sequencing approach is the ability to perform UMI matching and alignment to an original molecule. If a sequenced read does not contain at least one UMI, then it will typically be thrown out of a given data set, decreasing sequencing efficiency and depth. Using the randomer primer fragmentation method, all reads will contain at least one UMI by virtue of the randomer primer RT-PCR reaction that will generate dsDNA template polynucleotides incorporating at least the UMI at the 5′ end of the template polynucleotide. In some embodiments, the randomer primer will anneal downstream of the second UMI on the template polynucleotide, and a dsDNA template polynucleotide will be generated containing two UMIs. This approach therefore provides the significant advantage of ensuring that every read sequenced will be a usable read for downstream analysis.

### Method: Dual-UMI DNA Template Amplification and Random Fragmentation

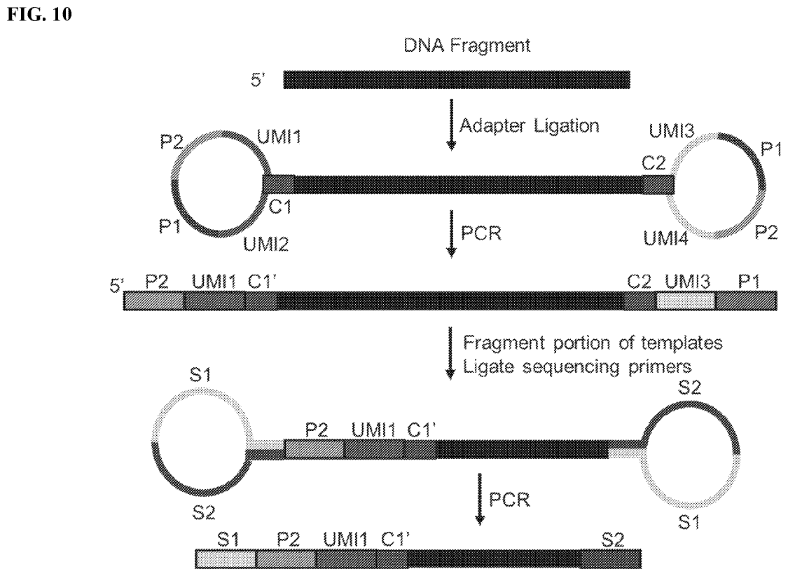

An alternate embodiment of the amplification and fragmentation method described supra employing hairpin adapters include two UMIs is described herein. FIG. 10 illustrates an alternate embodiment of a dual-UMI based amplification approach. First, adapter ligation (e.g., hairpin adapter ligation) on a DNA fragment is performed. Although only a single strand of the DNA fragment is shown, it will be understood that the hairpin adapters are ligated onto a double-stranded DNA nucleic acid fragment. Each hairpin adapter includes two UMI sequences (e.g., UMI1 and UMI2), two primer binding sequences (e.g., P1 and P2), and a duplexed constant region (e.g., C1/C2, shown as a single rectangle). Following adapter ligation, PCR amplification of the template polynucleotide is performed, such that the amplification product includes two UMI sequences. Although only the top strand with UMI1 and UMI3 is shown as amplified, it is understood that the bottom strand with UMI2 and UMI4 will also be amplified. Following PCR, a portion of the amplified templates are fragmented (e.g., physical fragmentation), such that some full-length amplified product is retained. End repair and A-tailing is then performed on both the fragmented and full-length templates. Next, adapters including platform primer sequences are ligated to both the fragmented and full-length templates. The adapters are shown as hairpin adapters, each including a sequence complementary to a sequencing platform, referred to as S1 and S2. Subsequently, the platform primer-containing ligation products are PCR amplified and sequenced. Subsequently, the surface primer-containing ligation products are then PCR amplified and sequenced.

Method: Dual-UMI DNA Template Rolling Circle Amplification (RCA)/Exponential Rolling Circle Amplification (eRCA) and Random Fragmentation

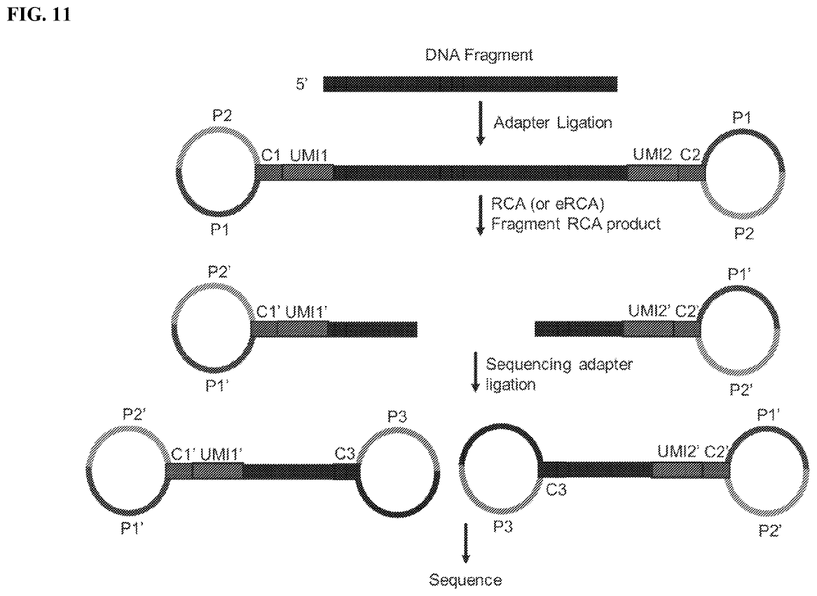

FIG. 11 illustrates an embodiment of a rolling circle amplification (RCA)-based approach for generating UMI-containing template polynucleotides for sequencing. Note that while RCA is indicated in the figure, any suitable circular amplification method (e.g., exponential rolling circle amplification (eRCA)) may be used. First, adapter ligation (e.g., hairpin adapter ligation) onto a DNA nucleic acid fragment is performed. Although only a single strand of the DNA nucleic acid fragment is shown, it is understood that in embodiments, the hairpin adapters are ligated onto a double-stranded DNA nucleic acid fragment. Each hairpin adapter includes a duplexed UMI sequence (e.g., UMI1, shown as a single rectangle), two primer binding sequences (e.g., P1 and P2), and a duplexed constant region (e.g., C1/C2, shown as a single rectangle). Rolling circle amplification (or alternatively, eRCA) is then performed using a strand-displacing polymerase (e.g., a phi29 DNA polymerase), followed by fragmentation of the RCA product and end-repair/A-tailing of the nucleic acid fragments. The nucleic acid fragments are then ligated to sequencing adapters (shown as hairpin adapters), wherein the sequencing adapters include a sequencing primer binding sequence (e.g., P3) and a duplexed constant region (e.g., a stem, referred to as C3, shown as a single rectangle). Following sequencing adapter ligation, the samples are then sequenced.

### Amplification and Sequencing

Current SBS platforms use clonal amplification of the initial template molecules with a cluster (i.e., polonies) to increase the signal-to-noise ratio because the systems are not sensitive enough to detect the extension of one base at the individual DNA template molecule level. Standard amplification methods employed in commercial sequencing devices (e.g., solid-phase bridge amplification) typically amplify a template using surface immobilized primers to produce a plurality of double-stranded nucleic acid molecules, wherein at least one strand of each double-stranded nucleic acid molecule is attached to the solid support at its 5′ ends. A common method of doing solid-phase amplification involves bridge amplification methodologies (referred to as bridge PCR) as exemplified by the disclosures of U.S. Pat. Nos. 5,641,658; 7,115,400; 7,790,418; U.S. Patent Publ. No. 2008/0009420, each of which is incorporated herein by reference in its entirety. In sum, bridge amplification methods allow amplification products (e.g., amplicons) to be immobilized on a solid support in order to form arrays comprised of colonies (or “clusters”) of immobilized nucleic acid molecules. Each cluster or colony on such an array is formed from a plurality of identical immobilized polynucleotide strands and a plurality of identical immobilized complementary polynucleotide strands. The products of solid-phase amplification reactions are referred to as “bridged” structures when formed by annealed pairs of immobilized polynucleotide strands and immobilized complementary strands, both strands being immobilized on the solid support at the 5′ end, preferably via a covalent attachment. During bridge PCR, additional chemical additives may be included in the reaction mixture, in which the DNA strands are denatured by flowing a denaturant such as formamide or NaOH with the DNA, which chemically denatures complementary strands. This is followed by washing out the denaturant and reintroducing a polymerase in buffer conditions that allow primer annealing and extension.

The resultant strand is then subjected to a nucleic acid sequencing reaction using any available sequencing technology. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing comprises a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template.

In embodiments, single-end or paired-end sequencing is performed. Paired-end sequencing may be performed, for example, using the methods described in U.S. Patent Application No. 63/147,167, which is incorporated herein by reference in its entirety. In embodiments of paired end sequencing, the first sequencing read being about 50 bases or less, and the second sequencing read being about 250 bases or less. In embodiments of paired end sequencing, the first sequencing read being about 100 bases or less, and the second sequencing read being about 200 bases or less. In embodiments of paired end sequencing, the first sequencing read being about 150 bases or less, and the second sequencing read being about 150 bases or less. In some embodiments, the first sequencing read is about 35 bases or less. In embodiments, the second sequencing read is about 500 bases or less. In embodiments, the second sequencing read is about 1000 bases or less. Once data is available from the sequencing reaction, initial processing (often termed “pre-processing”) of the sequences is typically employed prior to annotation. Pre-processing includes filtering out low-quality sequences, sequence trimming to remove continuous low-quality nucleotides, merging paired-end sequences, or identifying and filtering out PCR repeats using known techniques in the art. The sequenced reads may then be assembled and aligned using bioinformatic algorithms known in the art (see, FIG. 5).

### Example 3: Tandem Repeat Expansions

A short tandem repeat is a region of genomic DNA with multiple adjacent copies of short (e.g., 1-6 base) sequence units. These repeat regions are highly mutable due to replication errors that can occur during cell divisions and, importantly, over 30 human diseases are known to be caused by tandem repeat expansions or contractions (see, for example, Tang, H., Kirkness, E. F., Lippert, C., Biggs, W. H., Fabani, M., Guzman, E., et al. (2017). Profiling of short-tandem-repeat disease alleles in 12,632 human whole genomes. Am. J. Hum. Genet. 101, 700-715). Most of the disease-causing expansions are longer than the currently used NGS sequencing devices, making it virtually impossible to accurately assemble those regions of interest using typical sequencing methods.

Variability of the CGG tandem repeat in the 5′ untranslated region (UTR) of the fragile X mental retardation gene (FMR1) is associated with various disorders. Whereas most individuals in the general population have around 30 CGG repeats (<45 repeats), patients with fragile X syndrome carry large, full expansions sized above 200 repeats. The intermediate zone (45-54 repeats) exists, and although carriers of intermediate alleles are generally believed to be healthy, some reports have shown that these alleles might be associated with Parkinsonism and fragile X-associated tremor/ataxia syndrome. Complicating matters, researchers have found the presence, location, and quantity of AGG triplets interrupting the repeat can influence the risk of offspring inheriting a disease.

Sequencing can be used to determine the repeat size and the detection of the number of interrupting AGG units utilizing the barcodes as described herein. This data may be used clinically for improved genetic counselling for individuals weighing the risk of having a child with FXS. Another example where this technology described herein can be useful is the ATTCT repeat embedded in intron 9 of the Spinocerebellar ataxia type 10 gene (SCA10) (see, for example, McFarland K N, Liu J, Landrian I, Godiska R, Shanker S, Yu F, Farmerie W G, Ashizawa T. PLoS One. 2015; 10(8):e0135906). The presence of those interruptions influence the phenotype of SCA10 patients and hence knowing the exact repeat structure allows for better genotype-phenotype correlations.

To an isolated DNA (e.g., UTR of the fragile X mental retardation gene (FMR1) or intron 9 of the Spinocerebellar ataxia type 10 gene (SCA10)) fragmented amplification and sequencing (as described herein) is performed. Once data is available from the sequencing reaction, initial processing (often termed “pre-processing”) of the sequences is typically employed prior to annotation. Pre-processing includes filtering out low-quality sequences, sequence trimming to remove continuous low-quality nucleotides, merging paired-end sequences, or identifying and filtering out PCR repeats using known techniques in the art. The sequenced reads may then be assembled and aligned using bioinformatic algorithms known in the art (see, FIGS. 1 and 5).

### Example 4. Polymorphic Regions of HLA

Sequencing the human leukocyte antigen (HLA) region, or the human major histocompatibility complex (MHC), is crucial for diagnosing autoimmune disorders and selection of donors in organ and stem cell transplantation. Genes in the region can be highly polymorphic, HLA-B being the most variable with >2000 alleles. The high variability in sequence makes this region exceptionally difficult to map with traditional sequencing technology (see, for example, Trowsdale J, Knight J C. Annu Rev Genomics Hum Genet. 2013; 14:301-23).

HLA can be divided into three molecule classes and regions, termed class I, II and III. Considering the Class I genes are approximately 3 kb in length, entire alleles, not simply exons only, can be sequenced using the technology and methods described herein. Class II genes can exceed 10 kb making them more difficult, but still possible with this technology.

To an isolated DNA (e.g., HLA-B nucleic acid sequence) fragmented amplification and sequencing (as described herein) is performed. Once data is available from the sequencing reaction, initial processing (often termed “pre-processing”) of the sequences is typically employed prior to annotation. Pre-processing includes filtering out low-quality sequences, sequence trimming to remove continuous low-quality nucleotides, merging paired-end sequences, or identifying and filtering out PCR repeats using known techniques in the art. The sequenced reads may then be assembled and aligned using bioinformatic algorithms known in the art (see, FIGS. 1 and 5).

### Example 5: RNA Sequencing Poly(A) Tails

Sequencing RNA (e.g., mRNA, rRNA, and tRNA) allows transcriptome investigation and discovery, and provides useful insight informing scientists which genes are turned on in a cell, what their level of expression is, and at what times they are activated or shut off.

Polyadenylation (poly(A)) is a post-transcriptional modification of RNA found in all eukaryotic cells and in organelles, and is critical for nuclear export, stability, and translation control, but difficulties in globally measuring poly(A)-tail lengths have impeded greater understanding of poly(A)-tail function. Most eukaryotic mRNAs have poly(A) tails, which are added by a poly(A) polymerase following cleavage of the primary transcript during transcriptional termination. These tails are typically then truncated by deadenylases, and in some cases (e.g. animal oocytes, early embryos, or at neuronal synapses), the poly(A) tail can be re-extended by cytoplasmic poly(A) polymerases. Although poly(A) tails must exceed a minimal length to promote translation, the influence of tail length beyond this minimum is largely unknown. The prevailing view is that longer tails generally lead to increased translation, a theory derived from appending increasing lengths of synthetic poly(A) tails on Xenopus oocytes resulting in increased translation (see, for example, Barkoff et al EMBO J. 1998 Jun. 1; 17(11): 3168-3175). Additional supporting studies found this to be true in yeasts, however the general relationship between tail length and translational efficiency has not been reported outside of yeast, primarily because transcriptome-wide measurements have been unfeasible for longer-tailed mRNAs.

The length of the poly(A) tail is crucial for the transport of the mature mRNAs to the cytoplasm, their translation efficiency in certain developmental stages, and the quality control and degradation of mRNA. Recent studies suggest the average poly(A) tail length is approximately 30 nucleotides in yeast and approximately 50-100 nucleotides in mammalian and Drosophila cell lines (see, for example, Subtelny A O, Eichhom S W, Chen G R, Sive H, Bartel D P. Poly(A)-tail profiling reveals an embryonic switch in translational control. Nature 2014; 508:66-71). The poly(A) tail is a dynamic region of the mRNA that is controlled differently depending on a specific developmental stage. It has been shown that an increase in poly(A) polymerase activity is associated with poor prognosis in certain cancers (see, for example, Scorilas A. Crit Rev Clin Lab Sci 2002; 39:193-224) and hematological diseases, and therefore, an understanding and control of the poly(A) tail length may be a determinant factor in the development of some diseases.

Methods described herein provide a new method for sequencing poly(A) RNA in its entirety, including the transcription start site, the splicing pattern, the 3′ end and the poly(A) tail. This approach may be validated by northern blotting and high-resolution poly(A) tail assays (Hire-PAT). For example, starting with an RNA transcript, adapters may be ligated onto the 5′ and 3′ ends and in the presence of a non-strand displacing reverse transcriptase, a complement of the RNA transcript is used as the input polynucleotide and subjected to the long-read methods described herein.

The nucleic acid sample used for this experiment contains total RNA or mRNA, preferably purified RNA or mRNA, from an organism (e.g., human). Total RNA includes, but is not limited to, protein coding RNA also called coding RNA such as messenger RNA (mRNA) and non-protein coding RNA (non-coding RNA or ncRNA), such as ribosomal RNA (rRNA), transfer RNA (tRNA), micro RNA (miRNA), small interfering RNA (siRNA), piwi-interacting RNA (piRNA), small nuclear RNA (snRNA) and small nucleolar RNA (snoRNA). Each one of these RNA types may be used as input. Optionally, and preferably, the RNA will include a poly(A) tail, however the RNA molecule may not have a poly(A) tail (e.g., non-protein coding RNAs (ncRNA) such as ribosomal RNA (rRNA), transfer RNA (tRNA), micro RNA (miRNA), small interfering RNA (siRNA), piwi-interacting RNA (piRNA) and small nuclear RNA (snRNA)). For example, prokaryotic mRNA does not have a poly(A) tail. In RNA molecules that do not have a poly A tail, a poly(A) tail may be added synthetically (e.g. enzymatically) to validate these studies. In embodiments, a poly(A) tail is enzymatically added to the RNA molecule using known techniques in the art.

An isolated RNA molecule (e.g., mRNA), may be further purified and selected for polyadenylation utilizing known techniques in the art (e.g., by mixing RNA with poly(T) oligomers covalently attached to a substrate, such as magnetic beads). The RNA may be reverse transcribed (e.g., reverse transcription with a non-strand displacing RT) to cDNA, followed by a DNA polymerase-mediated second strand synthesis to yield an input DNA molecule. It is known that RNA representation bias can be introduced with the generation of cDNA; therefore it may be preferable to use the RNA as the template directly. However, it is known that the quantity of mRNA is orders of magnitude different than genomic DNA; therefore, either one may be used as input.

### Example 6: Metagenomics and Profiling of Bacteria

The study of bacterial phylogeny and taxonomy by analyzing the 16S rRNA gene has become popular among microbiologists due to the need to study the diversity and structure of microbiomes thriving in specific ecosystems. Due to its presence in almost all bacteria, the 16S rRNA gene is a core component of the 30S small subunit of prokaryotes. The 16S sequence contains ten conserved (C) regions that are separated by nine variable (V1-V9) regions, wherein the V regions are useful for taxonomic identification. Due to limitations in NGS platforms, the entirety of the 16S gene (approximately 1,500-1,800 bp) is difficult to accurately sequence.

Clever design of primers have been reported and used for amplifying specific V regions of 16S rRNA; for example, the third, fourth, and fifth variable regions (V3, V4 and V5 regions, respectively) have been used for studies where classification and understanding phylogenic relationships is important (see for example, Baker G. C., et al J. of Microbiological Methods, V55 (2003), 541-555; and Wang, Y., et al. (2014). PloS one, 9(3), e90053). While the information gained from sequencing the V3 or V4 region is valuable, no single variable region can differentiate among all bacteria. For example, the V1 region has been demonstrated to be particularly useful for differentiating among species in the genus Staphylococcus, whereas V2 distinguished among Mycobacterial species and V3 among Haemophilus species (Chakravorty, S., et al (2007). Journal of microbiological methods, 69(2), 330-339). It would therefore be very beneficial to be able to sequence the entirety of the 16S gene without having to a priori select appropriate primer sets. The methods described herein provide a new method for sequencing the 16S rRNA gene in its entirety, including the constant and nine variable regions. The methods allow for accurate species level determination by sequencing the entirety of the 16S gene.

### Example 7: Sequencing of Cancer Samples

Genomic profiling of tumors plays a critical role in personalized therapy and has become the gold standard in diagnosis and treatment of multiple cancer types. The genetic diversity in cancer genomes is complex and dynamic throughout cancer progression. Genome-wide aberrations in cancer include gene amplifications and deletions, inversions, translocations and somatic mutations (Shlien A and Malkin D; Genome Med. 2009 Jun. 16; 1(6):62; Hong J and Gresham D. Biotechniques. 2017 Nov. 1; 63(5):221-226). Importantly, these changes are the basis for changes in expression levels of many oncogenes and tumor suppressors. While somatic mutations and small deletions and rearrangements are readily detected with short sequencing reads, long range rearrangements like copy number variations of genes (CNVs) pose a challenge owing to their repetitive nature.

Numerous DNA microarray and NGS assays exist that can measure genome-wide copy number changes. Generally, NGS provides better base resolution, improved dynamic range and does not have the limitation of requiring a priori knowledge of the aberrant loci. However, CNV determination by NGS is by no means trivial and is limited by coverage uniformity and poor mapping of repetitive regions (Yamamoto et al; Hum Genome Var. 2016 Aug. 18; 3:16025; Valsesia et al. Front Genet. 2013 May 30; 4:92; Alkan et al. Nat Rev Genet. 2011 May; 12(5):363-76). CNV determination relies on applying a combination of paired-end and split read mapping, modeling read depth of healthy regions to identify insertions/deletions and de novo assembly. Aside from coverage issues introduced by the sequencing platform, many NGS library preparation protocols give rise to physical copy number changes. For instance, exome libraries utilize hybridization probes whose capture efficiencies depend on the GC content of targeted regions. More commonly, library protocols include a PCR amplification step, a method that may be prone to amplification bias, and can often overrepresent shorter amplicons with low sequence complexity (Kou et al. PLoS One. 2016 Jan. 11; 11(1):e0146638). Taipale and coworkers were among the first groups to demonstrate absolute molecule by tagging library fragments with UMIs (Kivioja et al. Nat Methods. 2011 Nov. 20; 9(l):72-4; Pflug and von Haeseler. Bioinformatics. 2018 Sep. 15; 34(18):3137-3144). Attaching a UMI to each DNA nucleic acid fragment prior to amplification makes each molecule unique. The central idea underlying read counting by UMIs is to count the number of distinct UMI sequences detected rather than attempting to count the number of reads. The identities of the UMIs are determined by sequencing. When enough sequences have been obtained, many UMI will have been observed multiple times and the number of original DNA molecules can be determined simply by counting the number of UMIs. Hereby care must be taken to sequence with appropriate coverage, however, it is not necessary to directly observe all UMIs since the number of unobserved UMIs can be estimated based on the distribution of the copy numbers of the observed UMIs.

Using the described UMI-containing barcodes for whole genome library preparation, will benefit cancer genome analysis in multiple ways. First, the fragmented UMI-containing reads and resulting longer reads will improve the mapping quality and assembly of repetitive regions. This will allow for more accurate assembly of regions with extensive gene amplifications. Second, each read will be quantifiable via the UMI, facilitating read depth modeling along the chromosomes. Third, the presence of the UMI will allow for distinguishing somatic mutations from mutations that are introduced during PCR (Fu et al. BMC Genomics. 2018 Jul. 13; 19(1):531). With these corrections, rare mutations with frequencies of 1-5% can be detected in heterogenous tissues.

### Example 8: Pseudogene Analysis and Determination

Homopolymeric nucleic acid regions are repetitive elements that present major logistical and computational challenges for assembling nucleic acid fragments produced by traditional sequencing technologies, especially considering that approximately two-thirds of the sequence of the human genome consists of repetitive units. For example, the human genome includes minisatellite regions, repetitive motifs ranging in length from about 10-100 base pairs and can be repeated about 5 to 50 times in the genome, and short tandem repeats (STR), regions ranging in length from about 1-6 base pairs and can be repeated about 5 to 50 times in the genome (e.g., the sequence TATA is a dinucleotide STR). Complicating matters, mutations often lead to the gain or loss of an entire repeat unit, and sometimes two or more repeats simultaneously, which can significantly burden traditional sequencing methodologies.

In embodiments, the methods described herein are useful at identifying a pseudogene. A pseudogene is a nucleic acid region that has high sequence similarity (homology) to a known gene but is nonfunctional, that is, a pseudogene does not produce a functional final protein product that the parent gene produces. Usually, the DNA sequences of a pseudogene and of its functional parent gene are about 65% to 100% identical, and typically accumulate more variants than their parent genes.

Due to the relatively short length of the fragments of nucleic acids used in conventional NGS technologies, ranging in length from 35 to 600 base pairs, many technologies may struggle with accurately distinguishing pseudogenes from the parent gene. For example, if sequence reads containing a pseudogene-derived variant are inappropriately mapped to the parent gene, it may result in a false positive variant call. Similarly, if a parent gene-derived variant is inappropriately mapped to the pseudogene, it may result in a false negative result.

Complicating matters, it is estimated that humans have greater than 10,000 pseudogenes (Pei, B. et al. (2012). Genome biology, 13(9), R51). The ability to differentiate a gene from a pseudogene depends on the degree of homology between the duplicated region and the parent gene. Generally, variants in genes sharing 90%-98% homology with a pseudogene are still accurately detected and mapped. However, when the homology is greater than 98%, accurate detection and mapping of pseudogenes is challenging. For example, the ABCC6, ADAMTSL2, ANKRD11, BMPR1A, SDHA, GBA, CORO1A, HYDIN, HBA1/HBA2, CHEK2, SMN1/SMN2, PMS2, and BRAF exon 18 genes are typically challenging to correctly identify from their pseudogenes. In embodiments, identifying a disruption in the sequence relative to the parent gene (e.g., a missing promotor, missing start codon, frameshift, premature stop codon, missing introns, or partial deletion) is a useful way of identifying a pseudogene. In embodiments, the methods described herein allow for determining the sequence of long templates comprising such repetitive sequences. This greatly facilitates accurate assembly of sequence reads to determine the overall template sequence and identification of a pseudogene.

To an isolated nucleic acid (e.g., a nucleic acid sequence containing a gene or pseudogene) fragmented amplification and sequencing (as described herein) is performed. Once data is available from the sequencing reaction, initial processing (often termed “pre-processing”) of the sequences is typically employed prior to annotation. Pre-processing includes filtering out low-quality sequences, sequence trimming to remove continuous low-quality nucleotides, merging paired-end sequences, or identifying and filtering out PCR repeats using known techniques in the art. The sequenced reads may then be assembled and aligned using bioinformatic algorithms known in the art (see, FIGS. 1 and 5).

### P-Embodiments

The present disclosure provides the following illustrative embodiments.

Embodiment P1. A method of sequencing a sample polynucleotide, the method comprising: a) contacting the sample polynucleotide with a composition and a polymerase, wherein said composition comprises a plurality of native DNA nucleotides and cleavable site nucleotides, thereby forming a plurality of amplification products, wherein said amplification products comprise a cleavable site nucleotide at a different position relative to each other; b) cleaving the amplification products at the cleavable site nucleotide to form a population of different-sized nucleic acid fragments comprising a 3′ end; c) ligating an adapter to the 3′ end of each of the population of different-sized nucleic acid fragments thereby forming adapter fragments, wherein the adapter comprises a sequencing primer binding sequence; d) binding said adapter fragments to immobilized primers on a solid support, and amplifying the adapter fragments to form colonies of immobilized polynucleotide fragments, wherein said amplifying comprises a plurality of cycles of primer extension, denaturation, and primer hybridization; and e) hybridizing a sequencing primer to one or more of the immobilized polynucleotide fragments within the colonies and incorporating one or more nucleotides into the sequencing primer with a polymerase; and detecting the one or more incorporated nucleotides thereby sequencing the sample polynucleotide.

Embodiment P2. A method of sequencing a sample polynucleotide comprising a promoter sequence, the method comprising: a) contacting the sample polynucleotide with a composition comprising a plurality of nucleotides and an RNA polymerase thereby forming a plurality of amplification products; b) contacting the sample polynucleotide with a composition comprising a plurality of randomer primer oligonucleotides and extending the randomer primer oligonucleotides with a reverse transcriptase to form a population of different-sized nucleic acid fragments, wherein each of the randomer primer oligonucleotides comprises a platform primer binding sequence; c) binding said nucleic acid fragments to an immobilized primer on a solid support, and amplifying the nucleic acid fragments to form colonies of immobilized polynucleotide fragments, wherein amplifying comprises a plurality of cycles of primer extension, denaturation, and primer hybridization; and d) hybridizing a sequencing primer to one or more of the immobilized polynucleotide fragments within the colonies and incorporating one or more nucleotides into the sequencing primer with a polymerase; and detecting the one or more incorporated nucleotides thereby sequencing the sample polynucleotide.

Embodiment P3. The method of Embodiment P1, wherein the plurality of native DNA nucleotides comprises a plurality of dATP nucleotides, dCTP nucleotides, dTTP nucleotides, and dGTP nucleotides.

Embodiment P4. The method of Embodiment P1, wherein prior to step d), said adapter fragments are not amplified in solution.

Embodiment P5. The method of Embodiment P1 or Embodiment P2, wherein the sample polynucleotide comprises a first adapter and a second adapter, wherein the first adapter is a Y-adapter, a hairpin adapter, a blunt-ended adapter, or an adapter comprising a single-strand overhang and the second adapter is a Y-adapter, a hairpin adapter, a blunt-ended adapter, or an adapter comprising a single-strand overhang.

Embodiment P6. The method of any one of Embodiment P1 to Embodiment P5, wherein the first adapter, the second adapter, or both the first adapter and the second adapter comprise a barcode sequence.

Embodiment P7. The method of Embodiment P6, wherein each adapter comprises, from 5′ to 3′, a barcode sequence, a primer binding site, and a promoter sequence.

Embodiment P8. The method of Embodiment P1, wherein the cleavable site comprises one or more deoxyuracil triphosphates (dUTPs), deoxy-8-oxo-guanine triphosphates (d-8-oxoGs), methylated nucleotides, or ribonucleotides.

Embodiment P9. The method of Embodiment P1, wherein the sample polynucleotide comprises a promoter sequence.

Embodiment P10. The method of Embodiment P9, wherein step a) comprises contacting the sample polynucleotide with a composition comprising a plurality of nucleotides and a promoter primer and transcribing the sample polynucleotide with an RNA polymerase thereby forming a plurality of amplification products.

Embodiment P11. The method of Embodiment P6, wherein each adapter comprises (i) a first strand comprising, from 5′ to 3, a barcode sequence, a first primer binding sequence, a second primer binding sequence, and a promoter sequence; and (ii) a second strand comprising, from 3′ to 5′, a sequence complementary to the barcode sequence, and a sequence complementary to the first primer binding sequence.

Embodiment P12. The method of Embodiment P6, wherein each adapter comprises, from 5′ to 3′, a barcode sequence, a primer binding sequence, a promoter sequence, a cleavable site, and a sequence complementary to the barcode sequence.

Embodiment P13. The method of Embodiment P6, wherein each adapter comprises, from 5′ to 3′, a first barcode sequence, a primer binding site, a promoter sequence, and a second barcode sequence.

Embodiment P14. The method of Embodiment P12 or Embodiment P13, wherein each adapter comprises an adapter cleavable site.

Embodiment P15. The method of any one of Embodiment P2, Embodiment P5 to Embodiment P7, or Embodiment P9 to Embodiment P14, wherein the randomer primer oligonucleotides comprise, from 3′ to 5′, a non-targeted template hybridization sequence and a platform primer sequence.

Embodiment P16. The method of any one of Embodiment P1 to Embodiment P15, wherein the sample polynucleotide is a double-stranded polynucleotide.

Embodiment P17. The method of any one of Embodiment P2, Embodiment P5 to Embodiment P7, or Embodiment P11 to Embodiment P16, wherein the reverse transcriptase is a strand-displacing reverse transcriptase.

Embodiment P18. The method of any one of Embodiment P15 to Embodiment P17, wherein the non-targeted template hybridization sequence is about 4 to about 30 nucleotides in length.

Embodiment P19. The method of any one of Embodiment P2 to Embodiment P18, wherein the promoter sequence is a T3 RNA polymerase promoter sequence, T5 RNA polymerase promoter sequence, or T7 RNA polymerase promoter sequence.

Embodiment P20. The method of claim any one of Embodiment P2 to Embodiment P19, wherein the method further comprises, prior to step b), fragmenting the plurality of amplification products to generate a plurality of polynucleotide fragments comprising 3′ ends, and ligating an adapter sequence to the 3′ end of each of the polynucleotide fragments.

Embodiment P21. The method of Embodiment P20, wherein the adapter comprises single-stranded RNA.

Embodiment P22. The method of Embodiment P20 or Embodiment P21, wherein the adapter sequence is ligated onto a single-stranded nucleic acid with a ligase, wherein the ligase is T4 RNA ligase.

Embodiment P23. The method of any one of Embodiment P1 to Embodiment P22, wherein prior to forming a population of different-sized nucleic acid fragments, an aliquot comprising the sample polynucleotide comprising at least a first adapter is retained.

Embodiment P24. The method of any one of Embodiment P1 to Embodiment P23, further comprising generating a sequencing read.

Embodiment P25. The method of Embodiment P24, wherein generating a sequencing read comprises executing a plurality of sequencing cycles, each cycle comprising extending the sequencing primer by incorporating a nucleotide or nucleotide analogue using a polymerase and detecting a characteristic signature indicating that the nucleotide or nucleotide analogue has been incorporated.

### Additional Embodiments

The present disclosure provides the following additional illustrative embodiments.

Embodiment 1. A method of sequencing a polynucleotide, the method comprising: contacting the polynucleotide comprising a first unique molecular identifier (UMI) sequence and a promoter sequence with an RNA polymerase and generating a plurality of RNA molecules, wherein each RNA molecule comprises a complement of said first UMI; fragmenting said plurality of RNA molecules to form a population of RNA nucleic acid fragments; attaching said population of RNA nucleic acid fragments to a solid support thereby forming a plurality of immobilized RNA nucleic acid fragments, and amplifying the plurality of immobilized RNA nucleic acid fragments to form amplification products immobilized to the solid support; hybridizing a sequencing primer to one or more of the amplification products and incorporating one or more nucleotides into the sequencing primer with a polymerase thereby forming one or more incorporated nucleotides; and detecting the one or more incorporated nucleotides thereby generating a sequencing read.

Embodiment 2. The method of Embodiment 1, further comprising attaching an adapter comprising a second UMI to said RNA nucleic acid fragments.

Embodiment 3. The method of Embodiment 2, further comprising sequencing the first UMI sequence and the second UMI sequence, thereby generating a plurality of sequencing reads, and grouping the plurality of sequencing reads based on co-occurrence of each of the UMI sequences.

Embodiment 4. The method of any one of Embodiments 1 to 3, wherein fragmenting said plurality of RNA molecules comprises contacting said plurality of RNA molecules with a plurality of oligonucleotide primers, and extending said plurality of oligonucleotide primers, wherein each oligonucleotide primer comprises a random sequence and a platform primer binding sequence.

Embodiment 5. The method of Embodiment 4, wherein each oligonucleotide primer comprises, from 5′ to 3′, the platform primer binding sequence and the random sequence.

Embodiment 6. The method of Embodiment 4 or 5, wherein the random sequence is about 4 to about 30 nucleotides in length.

Embodiment 7. The method of any one of Embodiments 1 to 6, comprising attaching an adapter comprising a primer binding sequence to each of said RNA nucleic acid fragments.

Embodiment 8. The method of any one of Embodiments 1 to 7, wherein generating a sequencing read comprises sequencing by synthesis, sequencing by ligation, sequencing-by-binding, or pyrosequencing.

Embodiment 9. The method of any one of Embodiments 1 to 7, wherein generating a sequencing read comprises executing a plurality of sequencing cycles, each cycle comprising extending the sequencing primer by incorporating a labeled nucleotide or labeled nucleotide analogue using a polymerase and detecting the label to generate a signal for each incorporated nucleotide or nucleotide analogue.

Embodiment 10. The method of any one of Embodiments 1 to 9, wherein the polynucleotide is a double-stranded polynucleotide.

Embodiment 11. The method of any one of Embodiments 1 to 10, wherein the promoter sequence is a T3 RNA polymerase promoter sequence, T5 RNA polymerase promoter sequence, or T7 RNA polymerase promoter sequence.

Embodiment 12. The method of any one of Embodiments 1 to 11, wherein the RNA polymerase is T7 RNA polymerase.

Embodiment 13. The method of any one of Embodiments 1 to 12, wherein amplifying comprises hybridizing an immobilized DNA oligonucleotide to the plurality of RNA nucleic acid fragments and extending the immobilized DNA oligonucleotide with a reverse transcriptase to form cDNA amplification products immobilized to the solid support.

Embodiment 14. The method of any one of Embodiments 1 to 13, wherein prior to attaching said population of RNA nucleic acid fragments to a solid support, the method further comprises amplifying said population of RNA nucleic acid fragments to generate a population of DNA nucleic acid fragments.

Embodiment 15. The method of Embodiment 14, further comprising hybridizing an immobilized DNA oligonucleotide to the DNA nucleic acid fragments and extending the immobilized DNA oligonucleotide with a polymerase to form amplification products immobilized to the solid support.

Embodiment 16. The method of any one of Embodiments 1 to 15, further comprising, prior to fragmenting, attaching a primer binding sequence to a full-length RNA molecule, amplifying said full-length RNA molecule to form full-length DNA molecules, and attaching said RNA nucleic acid fragments and full-length DNA molecules to the solid support.

Embodiment 17. The method of Embodiment 16, further comprising sequencing said full-length DNA molecules.

Embodiment 18. A method of sequencing a polynucleotide, the method comprising: a) contacting the polynucleotide with an amplification reagent and generating a first complement of said polynucleotide comprising an incorporated first cleavable site nucleotide at a first position; contacting the polynucleotide with said amplification reagent and generating a second complement of said polynucleotide comprising a second incorporated cleavable site nucleotide at a second position, wherein said first position and second position are different; wherein said amplification reagent comprises a polymerase, a plurality of native DNA nucleotides, and a plurality of cleavable site nucleotides; b) cleaving the first complement at the first position and cleaving the second complement at the second position to form nucleic acid fragments comprising a 3′ end; c) ligating an adapter to the 3′ end of each of the nucleic acid fragments thereby forming adapter fragments, wherein the adapter comprises a sequencing primer binding sequence; d) attaching said adapter fragments to immobilized primers on a solid support, and amplifying the adapter fragments to form amplification products immobilized to the solid support; and e) sequencing the amplification products, or complements thereof.

Embodiment 19. The method of Embodiment 18, wherein the plurality of native DNA nucleotides comprises a plurality of dATP nucleotides, a plurality of dCTP nucleotides, a plurality of dTTP nucleotides, and a plurality of dGTP nucleotides.

Embodiment 20. The method of any one of Embodiments 1 to 19, wherein the polynucleotide comprises a first adapter and a second adapter, wherein the first adapter is a Y-adapter, a hairpin adapter, a blunt-ended adapter, or an adapter comprising a single-strand overhang and the second adapter is a Y-adapter, a hairpin adapter, a blunt-ended adapter, or an adapter comprising a single-strand overhang.

Embodiment 21. The method of Embodiment 20, wherein the first adapter, the second adapter, or both the first adapter and the second adapter comprise a UMI sequence.

Embodiment 22. The method of Embodiment 20 or 21, wherein each adapter comprises, from 5′ to 3′, a UMI sequence, a primer binding site, and a promoter sequence.

Embodiment 23. The method of any one of Embodiments 18 to 22, wherein the cleavable site nucleotide is a deoxyuracil triphosphate (dUTP), a deoxy-8-oxo-guanine triphosphate (d-8-oxoG), a methylated nucleotide, or a ribonucleotide.

Embodiment 24. The method of any one of Embodiments 18 to 23, wherein the polynucleotide comprises a promoter sequence.

Embodiment 25. The method of Embodiment 24, wherein said amplification reagent comprises a primer complementary to said promoter sequence, and wherein said polymerase is an RNA polymerase and step a) comprises transcribing the polynucleotide with said RNA polymerase thereby forming a plurality of RNA amplification products.

Embodiment 26. The method of Embodiment 24 or 25, wherein the promoter sequence is a T3 RNA polymerase promoter sequence, T5 RNA polymerase promoter sequence, or T7 RNA polymerase promoter sequence.

Embodiment 27. The method of Embodiment 25 or 26, wherein the method further comprises, prior to step b), fragmenting the plurality of RNA amplification products to generate a plurality of RNA nucleic acid fragments, wherein said plurality of RNA nucleic acid fragments are comprise a 3′ end, and ligating said adapter sequence to the 3′ end of each of the plurality of RNA nucleic acid fragments.

Embodiment 28. The method of Embodiment 27, wherein the adapter comprises single-stranded RNA.

Embodiment 29. The method of Embodiment 27 or 28, wherein the adapter sequence is ligated onto a single-stranded nucleic acid with a ligase, wherein the ligase is T4 RNA ligase.

Embodiment 30. The method of any one of Embodiments 21 to 29, wherein each adapter comprises (i) a first strand comprising, from 5′ to 3′, a UMI sequence, a first primer binding sequence, a second primer binding sequence, and a promoter sequence; and (ii) a second strand comprising, from 3′ to 5′, a sequence complementary to the UMI sequence, and a sequence complementary to the first primer binding sequence.

Embodiment 31. The method of any one of Embodiments 21 to 29, wherein each adapter comprises, from 5′ to 3′, a UMI sequence, a primer binding sequence, a promoter sequence, a cleavable site, and a sequence complementary to the UMI sequence.

Embodiment 32. The method of any one of Embodiments 21 to 29, wherein each adapter comprises, from 5′ to 3′, a first UMI sequence, a primer binding site, a promoter sequence, and a second UMI sequence.

Embodiment 33. The method of Embodiment 32, wherein each adapter comprises a cleavable site.

Embodiment 34. The method of any one of Embodiments 18 to 33, wherein the polynucleotide is a double-stranded polynucleotide.

1. A method of sequencing a polynucleotide, the method comprising:
contacting the polynucleotide comprising a first unique molecular identifier (UMI) sequence and a promoter sequence with an RNA polymerase and generating a plurality of RNA molecules, wherein each RNA molecule comprises a complement of said first UMI;
fragmenting said plurality of RNA molecules to form a population of RNA nucleic acid fragments;
attaching said population of RNA nucleic acid fragments to a solid support thereby forming a plurality of immobilized RNA nucleic acid fragments, and amplifying the plurality of immobilized RNA nucleic acid fragments to form amplification products immobilized to the solid support;
hybridizing a sequencing primer to one or more of the amplification products and incorporating one or more nucleotides into the sequencing primer with a polymerase thereby forming one or more incorporated nucleotides; and
detecting the one or more incorporated nucleotides and generating a sequencing read.

2. The method of claim 1, further comprising attaching an adapter comprising a second UMI to said RNA nucleic acid fragments.

3. The method of claim 2, further comprising sequencing the first UMI sequence and the second UMI sequence, thereby generating a plurality of sequencing reads, and computationally grouping the plurality of sequencing reads based on co-occurrence of each of the UMI sequences.

4. The method of claim 1, wherein fragmenting said plurality of RNA molecules comprises contacting said plurality of RNA molecules with a plurality of oligonucleotide primers, and extending said plurality of oligonucleotide primers, wherein each oligonucleotide primer comprises a random sequence and a platform primer binding sequence.

5.-9. (canceled)

10. The method of claim 1, wherein the polynucleotide is a double-stranded polynucleotide comprising about 5,000 to about 50,000 bp.

11. The method of claim 1, wherein the promoter sequence is a T3 RNA polymerase promoter sequence, T5 RNA polymerase promoter sequence, or T7 RNA polymerase promoter sequence.

12. (canceled)

13. The method of claim 1, wherein amplifying comprises hybridizing an immobilized DNA oligonucleotide to the plurality of RNA nucleic acid fragments and extending the immobilized DNA oligonucleotide with a reverse transcriptase to form cDNA amplification products immobilized to the solid support.

14. The method of claim 1, wherein prior to attaching said population of RNA nucleic acid fragments to a solid support, the method further comprises amplifying said population of RNA nucleic acid fragments to generate a population of DNA nucleic acid fragments.

15. The method of claim 14, further comprising hybridizing an immobilized DNA oligonucleotide to the DNA nucleic acid fragments and extending the immobilized DNA oligonucleotide with a polymerase to form amplification products immobilized to the solid support.

16. The method of claim 1, further comprising, prior to fragmenting, attaching a primer binding sequence to a full-length RNA molecule, amplifying said full-length RNA molecule to form full-length DNA molecules, and attaching said RNA nucleic acid fragments and full-length DNA molecules to the solid support.

17. (canceled)

18. A method of sequencing a polynucleotide, the method comprising:
a) contacting the polynucleotide with an amplification reagent and generating a first complement of said polynucleotide comprising an incorporated first cleavable site nucleotide at a first position; contacting the polynucleotide with said amplification reagent and generating a second complement of said polynucleotide comprising a second incorporated cleavable site nucleotide at a second position, wherein said first position and second position are different; wherein said amplification reagent comprises a polymerase, a plurality of native DNA nucleotides, and a plurality of cleavable site nucleotides;
b) cleaving the first complement at the first position and cleaving the second complement at the second position to form nucleic acid fragments comprising a 3′ end;
c) ligating an adapter to the 3′ end of each of the nucleic acid fragments thereby forming adapter fragments, wherein the adapter comprises a sequencing primer binding sequence;
d) attaching said adapter fragments to immobilized primers on a solid support, and amplifying the adapter fragments to form amplification products immobilized to the solid support; and
e) sequencing the amplification products, or complements thereof.

19. (canceled)

20. The method of claim 18, wherein the polynucleotide comprises a first adapter and a second adapter, wherein the first adapter is a Y-adapter, a hairpin adapter, a blunt-ended adapter, or an adapter comprising a single-strand overhang and the second adapter is a Y-adapter, a hairpin adapter, a blunt-ended adapter, or an adapter comprising a single-strand overhang.

21. The method of claim 20, wherein the first adapter, the second adapter, or both the first adapter and the second adapter comprise a UMI sequence.

22. (canceled)

23. The method of claim 18, wherein the cleavable site nucleotide is a deoxyuracil triphosphate (dUTP), a deoxy-8-oxo-guanine triphosphate (d-8-oxoG), a methylated nucleotide, or a ribonucleotide.

24. The method of claim 18, wherein the polynucleotide comprises a promoter sequence.

25. The method of claim 24, wherein said amplification reagent comprises a primer complementary to said promoter sequence, and wherein said polymerase is an RNA polymerase and step a) comprises transcribing the polynucleotide with said RNA polymerase thereby forming a plurality of RNA amplification products.

26. The method of claim 24, wherein the promoter sequence is a T3 RNA polymerase promoter sequence, T5 RNA polymerase promoter sequence, or T7 RNA polymerase promoter sequence.

27. The method of claim 25, wherein the method further comprises, prior to step b), fragmenting the plurality of RNA amplification products to generate a plurality of RNA nucleic acid fragments, wherein said plurality of RNA nucleic acid fragments are comprise a 3′ end, and ligating said adapter sequence to the 3′ end of each of the plurality of RNA nucleic acid fragments.

28. (canceled)

29. (canceled)

30. The method of claim 21, wherein each adapter comprises (i) a first strand comprising, from 5′ to 3′, a UMI sequence, a first primer binding sequence, a second primer binding sequence, and a promoter sequence; and (ii) a second strand comprising, from 3′ to 5′, a sequence complementary to the UMI sequence, and a sequence complementary to the first primer binding sequence.

31. (canceled)

32. The method of claim 21, wherein each adapter comprises, from 5′ to 3′, a first UMI sequence, a primer binding site, a promoter sequence, and a second UMI sequence.

33. (canceled)

34. (canceled)
